(19) United States
(12) Patent Application Publication
(10) Pub. No.: US 2023/0183364 A1
(43) Pub. Date: Jun. 15, 2023

(54) Anti-IL13R-alpha2 Antibodies, Antigen-Binding Fragments and Uses Thereof

(71) Applicant: ELICERA THERAPEUTICS AB, Göteborg (SE)

(72) Inventors: Di YU, Storvreta (SE); Tina SARÉN, Uppsala (SE); Magnus ESSAND, Vänge (SE); Helena PERSSON LOTSHOLM, Sigtuna (SE); Camilla HOFSTRÖM, Upplands Väsby (SE); Yasmin ANDERSSON, Täby (SE); Juan ASTORGA WELLS, Stockholm (SE); Annette ROOS, Uppsala (SE); Vendela PARROW, Uppsala (SE)

(21) Appl. No.: 17/996,022

(22) PCT Filed: May 5, 2021
(86) PCT No.: PCT/SE2021/050419

§ 371 Date: Oct. 12, 2022

(30) Foreign Application Priority Data
May 15, 2020 (SE) 2050572-3

(51) Int. Cl. C07K 16/28; C07K 14/705; C07K 14/725; A61K 47/68; A61P 35/00; A61P 25/00; C07K 14/715

(52) U.S. Cl. CPC C07K 16/2866; C07K 14/70521; C07K 14/7051; C07K 14/70578; A61K 47/6803; A61K 47/6849; A61P 35/00; A61P 25/00; C07K 14/7155; A61K 2039/505

(57) ABSTRACT

The invention relates to antibodies and antigen-binding fragment thereof capable of binding to IL13Rα2. The antibodies and antigen-binding fragment thereof are in particular useful in constructing chimeric antigen receptors (CARs) and in CAR based immunotherapy for treatment of IL13Rα2-expressing cancer diseases.

### TECHNICAL FIELD

The present embodiments generally relate to anti-IL13Rα2 antibodies and antigen-binding fragments thereof, and uses thereof, in particular in cancer treatment.

### BACKGROUND

Immunotherapy has revolutionized cancer treatment. However, patients with glioblastoma, an aggressive form of brain cancer, have not yet gained from the breakthrough of immunotherapy and the disease remains lethal. Adoptive transfer of patient-derived T cells, engineered ex vivo to express a chimeric antigen receptor (CAR) directed against an antigen expressed on the surface of cancer cells, is a form of immunotherapy that is currently being intensely investigated. The artificial transmembrane CAR molecule consists of an extracellular antigen-binding moiety, typically a single-chain variable fragment (scFv) from an antibody, a hinge region, a transmembrane domain and intracellular signaling domains from the T cell receptor complex (CD3ζ) and from one or more T cell co-stimulatory molecules, e.g., CD28, 4-1BB. Thus, CAR T cell therapy combines the specificity of an antibody with the killing potency of a T lymphocyte. CAR T cells targeting CD19 are highly effective in treatment of refractory B-cell malignancies and approved for acute lymphoblastic leukemia and non-Hodgkin lymphoma both in the U.S. and Europe. CAR T cell products targeting solid tumors have not yet been approved. Similarly, the concept of using CAR molecule to engineer NK cells and macrophages, designated CAR NKs and CAR macrophages, has been explored as well.

Interleukin-13 receptor subunit alpha-2 (IL13Rα2), also known as cluster of differentiation 213A2 (CD213A2), is a cell membrane protein that binds interleukin-13 (IL-13). IL13Rα2 is closely related to IL13Rα1 (CD213A1), which forms a receptor complex with interleukin-4 receptor alpha (IL4Rα), a subunit that is shared by the IL-13 and IL-4 receptors. When IL-13 binds to the IL13Rα1/IL4Rα receptor complex, a signaling process is initiated that leads to activation of JAK1, STAT3 and STATE. In clear contrast, IL13Rα2 binds IL-13 with high affinity as a monomer and lacks a significant cytoplasmic domain and therefore does not appear to function as a signal mediator. IL13Rα2 is sparsely expressed in healthy human cells and tissues except for some expression in testis and pituitary gland. However, IL13Rα2 has been found to be selectively over-expressed in a variety of cancers, including glioblastoma, which makes it a target for cancer therapy.

CAR T cells directed against IL13Rα2 have been evaluated in a small clinical trial for patients with glioblastoma (Clin Cancer Res 2015, 21: 4062-4072). For one patient, the treatment led to sustained regression for a period of time until an IL13Rα2-negative tumor clone grew back (N Engl J Med 2016, 375: 2561-2569).

U.S. Pat. No. 9,914,909 discloses T cells expressing a CAR that includes an extracellular domain that includes IL-13 or a variant thereof that binds IL13Rα2, a transmembrane region, and an intracellular signaling domain. The CAR T cells are said to be useful in treatment of glioblastoma.

U.S. Pat. No. 9,868,788 discloses an antibody that binds specifically to a linear epitope spanning the extracellular portion of human IL13Rα2 and having at least 90% sequence identity with canine IL13Rα2. The antibody coupled to a chemotherapeutic agent is said to be useful in treatment of glioblastoma.

U.S. Pat. No. 10,308,719 discloses antibodies, and fragments thereof, binding to IL13Rα2 and CAR constructs comprising such antibody fragments and uses thereof in treatment of glioblastoma. The antibodies inhibit the interaction between IL13 and IL13Rα2. N-linked glycosylation of IL13Rα2 contributed to the interaction of the antibody to IL13Rα2.

Mol Cancer Ther 2008, 7(6): 1579-1587 discloses fusion of single-chain Fv (scFv) against IL-13Rα2 as obtained from a human scFv antibody phage library and Pseudomonas exotoxin (PE) to get anti-IL-13Rα2(scFv)-PE38 immunotoxin. The resulting immunotoxin, though, did not mediate higher antitumor activity compared to a previously developed immunotoxin in the form of a fusion between IL-13 and PE (IL-13-PE38). This was due to low affinity of the scFv portion of the immunotoxin to the target antigen.

There is still a need for an improvement in treatment of glioblastoma and other cancers characterized by overexpression of IL13Rα2.

### SUMMARY

It is a general objective to provide anti-IL13Rα2 antibodies and antigen-binding fragments thereof.

It is a particular objective to provide such anti-IL13Rα2 antibodies and antigen-binding fragments thereof useful in CAR based immunotherapy.

This and other objectives are met by embodiments as disclosed herein.

The present invention is defined in the independent claims. Further embodiments of the present invention are defined in the dependent claims.

An aspect of the embodiments relates to an antibody, or an antigen-binding fragment thereof, capable of binding to IL13Rα2.

In an embodiment, the antibody, or the antigen-binding fragment thereof, has specificity for an epitope within a beta sheet area of IL13Rα2 comprising a first beta strand of amino acid number 68 to 75 in IL13Rα2, a loop following the first beta strand, a second beta strand of amino acid numbers 101 to 109 in IL13Rα2, a loop preceding the second beta strand, and a third beta strand of amino acid numbers 124 to 128 in IL13Rα2.

In another embodiment, the antibody, or the antigen-binding fragment thereof, comprises a variable heavy (VH) domain complementarity determining region 1 (CDR1) comprising the amino acid sequence GFTFX₁X₂X₃X₄, wherein each Xₙ, n=1 . . . 4, is independently selected from the group consisting of G, A, S and Y. The antibody, or the antigen-binding fragment thereof, also comprises a VH domain CDR2 comprising the amino acid sequence IB₁B₂B₃B₄B₅B₆T, wherein each Bₘ, m=1 . . . 6, is independently selected from the group consisting of G, S and Y. The antibody, or the antigen-binding fragment thereof, further comprises a VH domain CDR3 comprising the amino acid sequence AR-Z_{H}-Z₁DY, wherein Z₁ is selected from the group consisting of F, M, I and L and Z_{H} represents an amino acid sequence selected from the group consisting of WRSTYGY (SEQ ID NO: 15), YGHYAYGSY (SEQ ID NO: 16), YSSSGWYYGF (SEQ ID NO: 17), TPYSAY (SEQ ID NO: 18), RYRSHRPGLS (SEQ ID NO: 19), FHPRYGY (SEQ ID NO: 20), GSYSHYGAHY (SEQ ID NO: 21), YYHYDYGYYY (SEQ ID NO: 22), YSPFY (SEQ ID NO: 3), RNYWEHGGGS (SEQ ID NO: 24), HHYGYYPPGSVYY (SEQ ID NO: 25), and VEYTYYGSEGSPV (SEQ ID NO: 26). The antibody, or the antigen-binding fragment thereof, additionally comprises a variable light (VL) domain CDR1 comprising the amino acid sequence QSISSY (SEQ ID NO: 12) and a VL domain CDR2 comprising the amino acid sequence AAS. The antibody, or the antigen-binding fragment thereof, further comprises a VL domain CDR3 comprising the amino acid sequence QQ-Z_{L}-T, wherein Z_{L} represents an amino acid sequence selected from the group consisting of TYYSPH (SEQ ID NO: 28), DYYLF (SEQ ID NO: 29), SYSTPY (SEQ ID NO: 30), FYSYPL (SEQ ID NO: 31), AFSPS (SEQ ID NO: 32), SYDTLL (SEQ ID NO: 33), ALSSLP (SEQ ID NO: 34), FSTRLS (SEQ ID NO: 35), GYSFPP (SEQ ID NO: 4), STYPF (SEQ ID NO: 37), YGSNPL (SEQ ID NO: 38), and RYNGLF (SEQ ID NO: 39).

The invention also relates to a CAR comprising an antigen recognition domain comprising an antibody, or an antigen-binding fragment thereof, according to the invention, a transmembrane domain and an intracellular signaling domain; a T cell receptor (TCR) complex comprising an antigen recognition domain comprising an antibody, or an antigen-binding fragment thereof, according to the invention; and a conjugate comprising an antibody, or an antigen-binding fragment thereof, according to the invention and an effector molecule.

The invention further relates to an epitope of IL13Rα2. The epitope is within a beta sheet area of IL13Rαc2 comprising a first beta strand of amino acid number 68 to 75 in IL13Rαc2, a loop following the first beta strand, a second beta strand of amino acid numbers 101 to 109 in IL13Rα2, a loop preceding the second beta strand, and a third beta strand of amino acid numbers 124 to 128 in IL13Rα2.

Further aspects also relates to a nucleic acid molecule encoding an antibody, or an antigen-binding fragment thereof, a CAR and/or a TCR-complex according to the invention; a vector comprising the nucleic acid molecule, a cell comprising an antibody, or an antigen-binding fragment thereof, a CAR, a TCR-complex, a nucleic acid and/or a vector according to the invention; and a pharmaceutical composition comprising an antibody, or an antigen-binding fragment thereof, a CAR, a TCR-complex, a conjugate, a nucleic acid molecule, a vector and/or a cell according to the invention, and a pharmaceutically acceptable carrier.

The invention also relates to an antibody, or an antigen-binding fragment thereof, a CAR, a TCR-complex, a conjugate, a nucleic acid molecule, a vector, a cell, and/or a pharmaceutical composition according to the invention for use as a medicament, and in particular for use in treating or delaying the onset of a IL13Rα2-expressing cancer disease.

The invention also relates to a method of identifying an IL13Rα2-positive cell. The method comprises contacting a biological sample with an antibody, or an antigen-binding fragment thereof according to the invention and measuring the amount of the antibody, or the antigen-binding fragment thereof, bound to at least one cell of the biological sample, thereby identifying the at least one cell as an IL13Rα2-positive cell.

The antibodies, and antigen-binding fragments thereof, of the embodiments bind specifically to an epitope on IL13Rα2 and retain highly specific and strong binding to IL13Rα2 also when converted into single-chain variable fragment (scFv) format. The CAR constructs generated based on antigen-binding fragments of antibodies of the embodiments displayed high cytotoxic capacity when used in CAR T cell immunotherapy and can, thereby, be used in treatment of IL13Rα2-expressing cancers.

### BRIEF DESCRIPTION OF THE DRAWINGS

The embodiments, together with further objects and advantages thereof, may best be understood by making reference to the following description taken together with the accompanying drawings, in which:

FIG. 1 shows enzyme-linked immunosorbent assay (ELISA) results. Bacterial supernatants of 1E10B9 scFv (B9 scFv) and mAb47 scFv (47 scFv) were screened for binding to human IL13Rα2 and a non-relevant protein (streptavidin) by ELISA at three different concentrations (diluted 2, 20 and 200 times). Two colonies (clone_1 and clone_2) were assessed for each of the two scFv. The streptavidin specific scFv G-strep-1 was included as reference. Binding was detected by an HRP-labeled anti-FLAG antibody and absorbance values were measured at 450 nm (y-axis). Reported values are averages of duplicates. Also, blank values have been subtracted (signal obtained from just adding media, without scFv).

FIG. 2 shows homogeneous time resolved fluorescence (HTRF) results. Bacterial supernatants of 1E10B9 scFv (X-ME107-B9) and mAb47 scFv (X-ME107-47) were screened for binding to IL13Rα2 by HTRF. A non-relevant scFv was included as a negative control, expected to bind to the non-relevant protein. The binding signal (665 nm) and background/noise signal (615 nm) was measured and the R-value, the ratio of the signals, calculated for each sample (y-axis). Reported values are averages of duplicates. Also, blank values have been subtracted (signal obtained from just adding media, without scFv).

FIG. 3 shows gel electrophoresis of 44 purified W-ME107 scFv and reference clone mAb47 scFv.

FIG. 4 shows ELISA results. Binding signal measured as absorbance at 450 nm (y-axis) of 44 W-ME107 scFv clones, reference clone mAb47 scFv and G-strep-1 scFv (y-axis) to human IL13Rα2-avi (open bar), human IL13Rα2-Fc (black bar), mouse IL13Rα2-Fc (dark gray bar), human IL13Rα1-Fc (upwards diagonal stripped bar), non-relevant protein (dot-filled bar) and streptavidin (light grey bar).

FIG. 5 shows results of scFv binding to cell lines. Selected scFv were incubated with either human glioblastoma cell line U-87MG (endogenously expressing high levels of hIL13Rα2), or human non-small cell lung cancer cells A549 (as negative control). The cells were further stained with anti-FLAG-PE antibody and analyzed in flow cytometry. Binding of scFv to cell are shown as mean fluorescent intensity (MFI, y-axis).

FIG. 6 shows surface plasmon resonance (SPR) sensorgrams of twelve W-ME107 scFv clones binding to IL13Rα2 with and without the presence of IL13. Also, sensorgrams of the anti-streptavidin scFv G-strep-1 (negative control, expected to bind to streptavidin) are included. No activity of purified clone 47 scFv was detected.

FIG. 7 shows sensorgrams of epitope binning experiment. 10 nM of hIL13Rα2-avi was injected over a clone mAb47 mIgG1 immobilized surface, as well as pre-incubated with ten times molar excess (100 nM) of human IL13, W-ME107-10, W-ME107-27, W-ME107-75 or W-ME107-117. As control samples, 10 nM hIL13Rα2-avi pre-incubated with ten times molar excess of BI-8 scFv (negative control) or clone47 mIgG1 (positive control) were also assessed.

FIG. 8 illustrates the residual plot from the HDX-MS mapping of W-ME107-117 on IL13αR2, with the traces depicting different time points, and the vertical lines representing the sum of the differences per peptide. Measurements between the two vertical lines in the plot area are not statistically significant (difference between the antigen alone or in the presence of the scFv did not exceed the replicate variance at 95% confidence level).

FIG. 9 shows ELISA results. Binding signals, absorbance at 450 nM (y-axis), were plotted against peptides and hIL13Rα2 antigens (x-axis). Binding of W-ME107-117 scFv (black bars) and W-ME107-75 (gray bars) were only detected towards hIL13Rα2.

FIG. 10 shows the structure of IL13Rα2 (light grey, residues 31-328) in a complex with IL-13 (black) (PDB code 3LB6) and highlights the epitope of W-ME107-117. The epitope sequences defined by HDX-MS have been colored dark grey and are situated on domain 1 of the receptor. The left panel shows the structure in cartoon representation where beta strands are represented as arrows and helices as spirals. The epitope sequences are found on one of the two beta sheets (strands 4, 3, 6, 7) and comprise residues in strands 3, 6, 7, and the loop regions between strands 3 and 4 and between strands 5 and 6, both loops are facing domain 2. The right panel shows the structure in surface representation, where IL-13 has been removed highlighting that the epitope region does not interact with the ligand. The IL-13 binding site is presented as a black circle.

FIG. 11 shows schematic representation of lentiviral constructs. The EF1α promoter was utilized to drive expression of the chimeric antigen receptor (CAR) containing selected scFv and intracellular T cell activation domains. T2A self-cleaving peptide was used to separate CAR construct and green fluorescent protein (GFP), which was used for detection.

FIG. 12 shows CAR T cell killing results. Luciferase-expressing U-87MG cells (expressing IL13Rα2) or Mel526 cells (not expressing IL13Rα2) were co-cultured with different CAR T cell constructs at effector to target ratios ranging from 0:1-25:1 for 24 hours. Viability of target cells was assessed by measuring luciferase activity. The relative viability of target cells (y-axis) in co-cultures was determined relative to untreated controls (only tumor cells). Assay was performed with biological and experimental duplicates. Values are shown as mean±SEM.

FIG. 13 shows proliferation of different CAR T cell constructs upon target recognition. CAR T cells were stained with violet dye and co-cultured with U-87MG cells for 4 days. Co-cultures with lovastatin served as non-replication control. CAR T cells replicated upon target recognition and were categorized according to number of cell divisions (0, 1, 2, 3 and 4 divisions or more). The figure shows percentage of CD3⁺GFP⁺ cells in divisions. The percentage was assessed by determining % of total CD3⁺GFP⁺ population within each division peak. Representative data is shown.

FIGS. 14A to 14E highlight the epitope sequences of W-ME107-10, W-ME107-27 and W-ME107-75 as found by HDX-MS. (A) The structure of IL13Rα2 (light grey) in a complex with IL-13 (black) (PDB code 3LB6) is rotated 180 degrees compared to the view in FIG. 10. The epitope sequences are colored dark grey with numbered residue boundaries and are all situated on domain 3 of the receptor with the exception of one sequence, which starts in domain 2. The structure is in cartoon representation. (B-E) show IL13Rα2 in surface representation and highlight the differences in epitopes expected for W-ME107-10, W-ME107-27 and W-ME107-75 based on HDX-MS data together with binding data. Epitope areas are colored dark grey. IL-13 (black) is kept in cartoon representation for clarity. (B) and (C) illustrate the predicted binding areas of W-ME107-10 and W-ME107-27, respectively. In (D) the binding area of W-ME107-75 is shown in dark gray. W-ME107-75 behaves differently to W-ME107-10 and W-ME107-27 in many of the experiments and is therefore expected to have a different binding site. The epitope includes the very C-terminal part of the receptor, residues 329-337. These residues are not visible in the published structure but are included as a dashed line in the figure. They are presumed to be important in W-ME107-75 binding as they are non-conserved in mouse IL13Rα2. (E) Here the protein structure has been rotated 180 degrees and the start of peptide 228-245, which is part of the epitope for W-ME107-10 and W-ME107-27 but not W-ME107-75, is seen as dark grey.

FIGS. 15A to 15D show profiling and characterization of engineered CAR T cells. (A) shows IFN-gamma secretion into culture medium of unstimulated control (mock) CAR T cells, W-ME107-10 CAR T cells, W-ME107-27 CAR T cells, W-ME107-55 CAR T cells, W-ME107-75 CAR T cells and W-ME107-117 CAR T cells. (B) shows IFN-gamma secretion into culture medium of control (mock) CAR T cells, W-ME107-10 CART cells, W-ME107-27 CART cells, W-ME107-55 CART cells, W-ME107-75 CAR T cells and W-ME107-117 CAR T cells co-cultured with U87UU or U343MG tumor cells. (C) shows CAR expression in control (mock) CAR T cells, W-ME107-10 CAR T cells, W-ME107-27 CAR T cells, W-ME107-55 CAR T cells, W-ME107-75 CAR T cells and W-ME107-117 CAR T cells over time. (D) shows surface activating markers (PD-1, TIM-3, LAG-3, CD69 and CD25) on control (Mock) CAR T cells, W-ME107-27 CART cells, and W-ME107-117 CART cells in the presence or absence of tumor cell stimulation.

FIGS. 16A to 16C show that engineered CAR T cells control glioblastoma tumor growth in vivo. (A) schematic of experimental procedure. (B) show tumor growth at various days after tumor implantation for control (mock) CAR T cells, W-ME107-10 CAR T cells, W-ME107-75 CAR T cells and W-ME107-117 CAR T cells. (C) shows percent survival of the mice at various days after tumor implantation for control (mock) CART cells, W-ME107-10 CART cells, W-ME107-75 CART cells and W-ME107-117 CART cells.

FIG. 17A to 17B show that different complementary determining regions (CDR) regions of the heavy and light chain of the scFv affect CAR expression of CAR-T cells. (A) The CAR expression on human Jurkat cells for each construct. (ns: no statistic significant; *: P<0.05; **: P<0.01). (B) Representative plots showing GFP signal, as indication for transduced cell and CAR surface staining signal in each construct.

FIG. 18A to 18C described that basal level activation of engineered CAR T cells was diminished when CAR intracellular signaling domains were removed. (A) Schematic illustration of lentiviral constructs used to express CAR or decoy CAR (the CAR molecule without intracellular signaling domain named dCAR). (B) Schematic drawing depicts a CAR molecule and a decoy CAR molecule on the cell membrane. (C) IFN-γ secretion from different CAR-T cell constructs at day 7 after transduction without stimulation.

### DETAILED DESCRIPTION

The present embodiments generally relate to anti-IL13Rα2 antibodies and antigen-binding fragments thereof, and use thereof, in particular in cancer treatment.

The present embodiments relates to antibodies and antigen-binding fragments thereof having specificity for interleukin-13 receptor subunit alpha-2 (IL13Rα2). The antibodies and the antigen-binding fragments of the embodiments are particularly suitable to be used in chimeric antigen receptors (CARs) and in CAR based immunotherapy of various diseases, including cancer diseases characterized by expression and presentation of IL13Rα2.

Antibodies and CAR constructs against IL13Rα2 are known in the art as exemplified in the documents cited in the background section. However, the prior art solutions suffer from various shortcomings that limit their use in cancer treatment. For instance, the monoclonal anti-IL13Rα2 antibody 1E10B9 (U.S. Pat. No. 9,868,788; Debinski, et al., New agents for targeting of IL-13RA2 expressed in primary human and canine brain tumors, PLoS One 2013, 8(10): e77719)) lost its capability to specifically bind to human IL13Rα2 when converted from IgG antibody into single-chain variable fragment (scFv) format as shown in the Example section. Loss of antigen binding when alternating between antibody formats is not uncommon, especially when going from IgG to scFv. An altered protein fold affecting the structure of the antigen-binding site can most likely explain this. Hence, the prior art monoclonal anti-IL13Rα2 antibody 1E10B9 could be used in IgG form but is not suitable for conversion into the scFv format and is thereby not suitable for usage in CAR based immunotherapy.

The monoclonal anti-IL13Rα2 antibody mAb47 (U.S. Pat. No. 10,308,719; Balyasnikova et al., Characterization and immunotherapeutic implications for a novel antibody targeting interleukin (IL)-13 receptor α2, J Biol Chem 2012, 287(36): 30215-30227; Kim et al., A novel single-chain antibody redirects adenovirus to IL13Rα2-expressing brain tumors, Sci Rep 2015, 5: 18133) showed retained binding to human IL13Rα2 when converted into scFv format. However, in comparative examples with scFv of the present invention in CAR based immunotherapy, the CAR T cells generated based on mAb47 scFv showed only marginal target cell killing, whereas CAR T cells generated according to the embodiments displayed substantial cytotoxic capacity already at low effector to target cell ratios. Hence, antigen-binding fragments of the antibodies according to the embodiments are superior to the antigen-binding fragment of the antibody mAb47 when used in CAR based immunotherapy. Furthermore, the antibody mAb47 competes with the ligand interleukin-13 (IL-13) when binding to the receptor IL13Rα2, whereas several antibodies and antigen-binding fragments thereof of the embodiments do not compete with IL-13 for binding to IL13Rα2.

Herein, all amino acids in the variable regions of antibodies, or antigen-binding fragments thereof, including the herein described complementarity determining regions (CDRs), are consequently numbered and defined according to the International ImMunoGeneTics (IMGT) information system and nomenclature (Lefranc et al., Dev Comp Immunol. (2003) 1: 55-77).

Amino acid numbers in the human IL13Rα2 protein sequence is according to NCBI reference sequence with accession no. NP_000631 and version NP_000631.1 dated 26 Apr. 2021 and further presented here below (SEQ ID NO: 107).

MAFVCLAIGC LYTFLISTTF GCTSSSDTEI KVNPPQDFEI
VDPGYLGYLY LQWQPPLSLD HFKECTVEYE LKYRNIGSET
WKTIITKNLH YKDGFDLNKG IEAKIHTLLP WQCTNGSEVQ
SSWAETTYWI SPQGIPETKV QDMDCVYYNW QYLLCSWKPG
IGVLLDTNYN LFYWYEGLDH ALQCVDYIKA DGQNIGCRFP
YLEASDYKDF YICVNGSSEN KPIRSSYFTF QLQNIVKPLP
PVYLTFTRES SCEIKLKWSI PLGPIPARCF DYEIEIREDD
TTLVTATVEN ETYTLKTTNE TRQLCFVVRS KVNIYCSDDG
IWSEWSDKQC WEGEDLSKKT LLRFWLPFGF ILILVIFVTG
LLLRKPNTYP KMIPEFFCDT

The specificity of an antibody, or an antigen-binding fragment thereof, can be determined based on affinity and/or avidity. The affinity, represented by the equilibrium constant for the dissociation of an antigen with the antibody, or the antigen-binding fragment thereof, (K_{D}), is a measure for the binding strength between an antigenic determinant, i.e., epitope, and an antigen-binding site on the antibody, or the antigen-binding fragment thereof. The lesser the value of K_{D} the stronger the binding strength between the antigenic determinant and the antibody, or the antigen-binding fragment thereof. Alternatively, the affinity can also be expressed as the affinity constant (K_{A}), which is 1/K_{D}. As will be clear to the skilled person, affinity can be determined in a manner known per se, depending on the specific antigen of interest.

Avidity is the measure of the strength of binding between an antibody, or an antigen-binding fragment thereof, and the pertinent antigen. Avidity is related to both the affinity between an antigenic determinant and its antigen binding site on the antibody, or the antigen-binding fragment thereof, and the number of pertinent binding sites present on the antibody, or the antigen-binding fragment thereof.

Typically, antibodies, or antigen-binding fragments thereof, will bind to their antigen with an equilibrium dissociation constant (K_{D}) of 10⁻⁵ to 10⁻¹² moles/liter (M) or less, and preferably 10⁻⁷ to 10⁻¹² M or less and more preferably 10⁻⁸ to 10⁻¹² M, i.e. with an affinity constant (K_{A}) of 10⁵ to 10¹² M⁻¹ or more, and preferably 10⁷ to 10¹² M⁻¹ or more and more preferably 10⁸ to 10¹² M⁻¹.

Generally, any K_{D} value greater than 10⁻⁴ M (or any K_{A} value lower than 10⁴ M⁻¹) is considered to indicate non-specific binding.

Preferably, an antibody, or an antigen-binding fragment thereof, of the embodiments will bind to IL13Rα2 with an affinity less than 500 nM, preferably less than 200 nM, more preferably less than 10 nM, such as less than 5 nM.

Specific binding of an antibody, or an antigen-binding fragment thereof, to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, LUMINEX® Multiplex Assay and the different variants thereof known per se in the art.

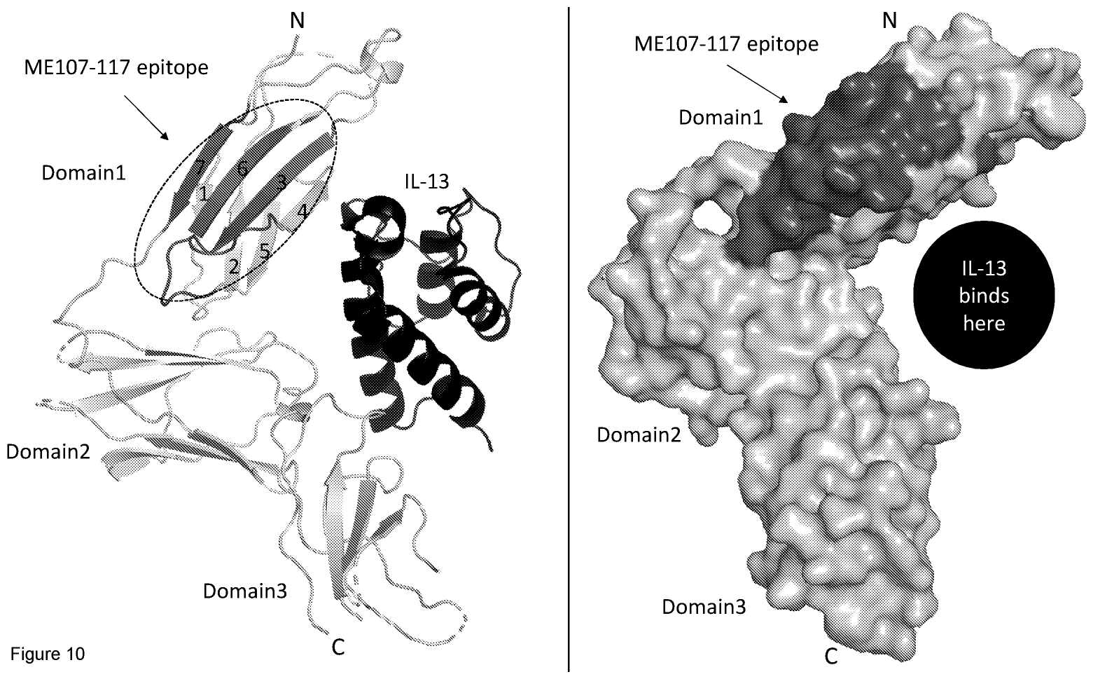

The present inventors have found a novel epitope or antigenic determinant region in IL13Rα2, which is highly suitable for targeting by antibodies and antigen-binding fragment thereof. In particular, antibodies and antigen-binding fragments thereof targeting this novel epitope are useful in CAR based immunotherapy. This epitope corresponds to the second and largest beta sheet in domain 1 of IL13Rα2 as shown in FIG. 10. This is the N-terminal part of the receptor and domain 1 consists of a beta sandwich fold with two beta sheets on top of each other. The epitope is located in 3 of the 4 strands in the largest beta sheet and is in the area pointing away from the binding site of IL-13 to IL13Rα2, see FIG. 10.

Antibodies and antigen-binding fragments thereof binding specifically to an epitope in this beta sheet area had high specificity for IL13Rα2, showed no binding to IL13Rα1. The antigen-binding fragments showed superior cytotoxic capacity to cancer cells expressing IL13Rα2 in CAR T cell based immunotherapy.

An aspect of the embodiments therefore relates to an antibody, or an antigen-binding fragment thereof, capable of binding to IL13Rα2. The antibody, or the antigen-binding fragment thereof, has specificity for an epitope within a beta sheet area or domain of IL13Rα2 comprising a first beta strand of amino acid number 68 to 75 in IL13Rα2, a loop following the first beta strand, a second beta strand of amino acid numbers 101 to 109 in IL13Rα2, a loop preceding the second beta strand, and a third beta strand of amino acid numbers 124 to 128 in IL13Rα2.

The amino acid sequence of IL13Rα2 is presented in SEQ ID NO: 107. Amino acid number 68 to 75 correspond to the amino acid sequence EYELKYRN (SEQ ID NO: 108), amino acid number 101 to 109 correspond to the amino acid sequence IEAKIHTLL (SEQ ID NO: 109) and amino acid number 124 to 128 correspond to the amino acid sequence AETTY (SEQ ID NO: 110).

FIG. 10 illustrates the largest beta sheet of domain 1 with a hatched ellipse surrounding the three beta strands, numbered 3, 6 and 7, forming an epitope. The beta strands 6 and 7 are interconnected with a loop region that contains an alpha helix and a bend. Beta strand 3 and 6 are interconnected with an amino acid sequence comprising two turns and two beta strands (number 4 and 5, where beta strand 4 belongs to the largest beta sheet together with strands 3, 6, 7, and beta strand 5 belongs to the first smaller beta sheet of domain 1).

In an embodiment, the antibody, or the antigen-binding fragment thereof, has specificity for an epitope comprising at least one peptide, also referred to as epitope region, selected from the group consisting of amino acid number 67 to 81, amino acid number 96 to 106 and amino acid number 123 to 128 in IL13Rα2.

In an embodiment, the first peptide or epitope region (VEYELKYRNIGSETW, SEQ ID NO: 44) substantially corresponds to beta strand 3 in IL13Rα2 and the turn following beta strand 3. The second peptide or epitope region (DLNKGIEAKIH, SEQ ID NO: 45) substantially corresponds to beta strand 6 and a short stretch of the loop preceding it, whereas the third peptide or epitope region (WAETTY, SEQ ID NO: 46) substantially corresponds to beta strand 7.

The antibody, or the antigen-binding fragment thereof, has specificity for at least one of these three peptides or epitope regions in the beta sheet area of IL13Rα2. Hence, the antibody, or the antigen-binding fragment thereof, has specificity for the first peptide or epitope region (VEYELKYRNIGSETW, SEQ ID NO: 44), the antibody, or the antigen-binding fragment thereof, has specificity for the second peptide or epitope region (DLNKGIEAKIH, SEQ ID NO: 45) or the antibody, or the antigen-binding fragment thereof, has specificity for the third peptide or epitope region (WAETTY, SEQ ID NO: 46).

In an embodiment, the antibody, or the antigen-binding fragment thereof, has specificity for at least two of these three peptides or epitope regions in the beta sheet area of IL13Rα2. Hence, the antibody, or the antigen-binding fragment thereof, has specificity for the first peptide or epitope region (VEYELKYRNIGSETW, SEQ ID NO: 44) and the second peptide or epitope region (DLNKGIEAKIH, SEQ ID NO: 45), the antibody, or the antigen-binding fragment thereof, has specificity for the first peptide or epitope region (VEYELKYRNIGSETW, SEQ ID NO: 44) and the third peptide or epitope region (WAETTY, SEQ ID NO: 46) or the antibody, or the antigen-binding fragment thereof, has specificity for the second peptide or epitope region (DLNKGIEAKIH, SEQ ID NO: 45) and the third peptide or epitope region (WAETTY, SEQ ID NO: 46).

In a preferred embodiment, the antibody, or the antigen-binding fragment thereof, has specificity for all of these three peptides or epitope regions in the beta sheet area of IL13Rα2, i.e., the first peptide or epitope region (VEYELKYRNIGSETW, SEQ ID NO: 44), the second peptide or epitope region (DLNKGIEAKIH, SEQ ID NO: 45) and the third peptide or epitope region (WAETTY, SEQ ID NO: 46).

The invention also relates to an epitope of IL13Rα2. The epitope is within a beta sheet area of IL13Rα2 comprising a first beta strand of amino acid number 68 to 75 in IL13Rα2, a loop following the first beta strand, a second beta strand of amino acid numbers 101 to 109 in IL13Rα2, a loop preceding the second beta strand, and a third beta strand of amino acid numbers 124 to 128 in IL13Rα2.

In an embodiment, the epitope comprises at least one peptide, preferably at least two peptides, and more preferably all three peptides, selected from the group consisting of amino acid number 67 to 81, i.e., VEYELKYRNIGSETW (SEQ ID NO: 44), amino acid number 96 to 106, i.e., DLNKGIEAKIH (SEQ ID NO: 45), and amino acid number 123 to 128, i.e., WAETTY (SEQ ID NO: 46), in IL13Rα2.

In an embodiment, the antibody, or the antigen-binding fragment thereof, comprises a variable heavy (VH) domain complementarity determining region 3 (CDR3) comprising the amino acid sequence YSPFY (SEQ ID NO: 3).

In an embodiment, the antibody, or the antigen-binding fragment thereof, comprises, a VH domain CDR3 comprising the amino acid sequence YSPFYM (SEQ ID NO: 9). In a particular embodiment, the antibody, or the antigen-binding fragment thereof, comprises a VH domain CDR3 comprising, preferably consisting of, the amino acid sequence ARYSPFYMDY (SEQ ID NO: 10).

In an embodiment, the antibody, or the antigen-binding fragment thereof, comprises a variable light (VL) domain CDR3 comprising the amino acid sequence GYSFPP (SEQ ID NO: 4).

In a particular embodiment, the antibody, or the antigen-binding fragment thereof, comprises a VL CDR3 comprising, preferably consisting of, the amino acid sequence QQGYSFPPT (SEQ ID NO: 11).

In an embodiment, the antibody, or the antigen-binding fragment thereof, comprises a VH domain CDR1 comprising the amino acid sequence SGSY (SEQ ID NO: 1).

In an embodiment, the antibody, or the antigen-binding fragment thereof, comprises a VH domain CDR1 comprising, preferably consisting of, the amino acid sequence GFTFSGSY (SEQ ID NO: 106).

In an embodiment, the antibody, or the antigen-binding fragment thereof, comprises an extended VH domain CDR1 comprising the amino acid sequence SGSYMS (SEQ ID NO: 5), preferably comprising, preferably consisting of, the amino acid sequence GFTFSGSYMS (SEQ ID NO: 6).

In an embodiment, the antibody, or the antigen-binding fragment thereof, comprises a VH domain CDR2 comprising the amino acid sequence YGSGGY (SEQ ID NO: 2).

In a particular embodiment, the antibody, or the antigen-binding fragment thereof, comprises a VH domain CDR2 comprising, preferably consisting of, the amino acid sequence IYGSGGYT (SEQ ID NO: 7).

In an embodiment, the antibody, or the antigen-binding fragment thereof, comprises an extended VH domain CDR2 comprises, preferably consists of, the amino acid sequence SIYGSGGYTY (SEQ ID NO: 8).

Extended CDR as used herein relates to an amino acid sequence that comprises at least one additional amino acid residue beyond the amino acids of the CDR as defined according to the IMGT nomenclature.

In an embodiment, the antibody, or the antigen-binding fragment thereof, comprises a VL domain CDR1 comprising, preferably consisting of, the amino acid sequence QSISSY (SEQ ID NO: 12).

In an embodiment, the antibody, or the antigen-binding fragment thereof, comprises a VL domain CDR2 comprising, preferably consisting of, the amino acid sequence AAS.

The antibody, or the antigen-binding fragment thereof, can comprise at least one of the above described embodiments of VH domain and/or VL domain CDR regions, preferably at least two, more preferably at least three, and even more preferably at least four, at least five or all of the CDR regions.

In an embodiment, the antibody, or the antigen-binding fragment thereof, comprises at least two of the above described VH domain and/or VL domain CDR regions. Such embodiments include an antibody, or an antigen-binding fragment thereof, comprising a VH domain CDR1 and a VH domain CDR2 as defined above; an antibody, or an antigen-binding fragment thereof, comprising a VH domain CDR1 and a VH domain CDR3 as defined above; an antibody, or an antigen-binding fragment thereof, comprising a VH domain CDR1 and a VL domain CDR1 as defined above; an antibody, or an antigen-binding fragment thereof, comprising a VH domain CDR1 and a VL domain CDR2 as defined above; an antibody, or an antigen-binding fragment thereof, comprising a VH domain CDR1 and a VL domain CDR3 as defined above; an antibody, or an antigen-binding fragment thereof, comprising a VH domain CDR2 and a VH domain CDR3 as defined above; an antibody, or an antigen-binding fragment thereof, comprising a VH domain CDR2 and a VL domain CDR1 as defined above; an antibody, or an antigen-binding fragment thereof, comprising a VH domain CDR2 and a VL domain CDR2 as defined above; an antibody, or an antigen-binding fragment thereof, comprising a VH domain CDR2 and a VL domain CDR3 as defined above; an antibody, or an antigen-binding fragment thereof, comprising a VH domain CDR3 and a VL domain CDR1 as defined above; an antibody, or an antigen-binding fragment thereof, comprising a VH domain CDR3 and a VL domain CDR2 as defined above; an antibody, or an antigen-binding fragment thereof, comprising a VH domain CDR3 and a VL domain CDR3 as defined above; an antibody, or an antigen-binding fragment thereof, comprising a VL domain CDR1 and a VL domain CDR2 as defined above; an antibody, or an antigen-binding fragment thereof, comprising a VL domain CDR1 and a VL domain CDR3 as defined above; and an antibody, or an antigen-binding fragment thereof, comprising a VL domain CDR2 and a VL domain CDR3 as defined above.

In an embodiment, the antibody, or the antigen-binding fragment thereof, comprises at least three of the above described VH domain and/or VL domain CDR regions. Such embodiments include an antibody, or an antigen-binding fragment thereof, comprising a VH domain CDR1, a VH domain CDR2 and a VH domain CDR3 as defined above; an antibody, or an antigen-binding fragment thereof, comprising a VH domain CDR1, a VH domain CDR2 and a VL domain CDR1 as defined above; an antibody, or an antigen-binding fragment thereof, comprising a VH domain CDR1, a VH domain CDR2 and a VL domain CDR2 as defined above; an antibody, or an antigen-binding fragment thereof, comprising a VH domain CDR1, a VH domain CDR2 and a VL domain CDR3 as defined above; an antibody, or an antigen-binding fragment thereof, comprising a VH domain CDR1, a VH domain CDR3 and a VL domain CDR1 as defined above; an antibody, or an antigen-binding fragment thereof, comprising a VH domain CDR1, a VH domain CDR3 and a VL domain CDR2 as defined above; an antibody, or an antigen-binding fragment thereof, comprising a VH domain CDR1, a VH domain CDR3 and a VL domain CDR3 as defined above; an antibody, or an antigen-binding fragment thereof, comprising a VH domain CDR1, a VL domain CDR1 and a VL domain CDR2 as defined above; an antibody, or an antigen-binding fragment thereof, comprising a VH domain CDR1, a VL domain CDR1 and a VL domain CDR3 as defined above; an antibody, or an antigen-binding fragment thereof, comprising a VH domain CDR1, a VL domain CDR2 and a VL domain CDR3 as defined above; an antibody, or an antigen-binding fragment thereof, comprising a VH domain CDR2, a VH domain CDR3 and a VL domain CDR1 as defined above; an antibody, or an antigen-binding fragment thereof, comprising a VH domain CDR2, a VH domain CDR3 and a VL domain CDR2 as defined above; an antibody, or an antigen-binding fragment thereof, comprising a VH domain CDR2, a VH domain CDR3 and a VL domain CDR3 as defined above; an antibody, or an antigen-binding fragment thereof, comprising a VH domain CDR2, a VL domain CDR1 and a VL domain CDR2 as defined above; an antibody, or an antigen-binding fragment thereof, comprising a VH domain CDR2, a VL domain CDR1 and a VL domain CDR3 as defined above; an antibody, or an antigen-binding fragment thereof, comprising a VH domain CDR2, a VL domain CDR2 and a VL domain CDR3 as defined above; an antibody, or an antigen-binding fragment thereof, comprising a VH domain CDR3, a VL domain CDR1 and a VL domain CDR2 as defined above; an antibody, or an antigen-binding fragment thereof, comprising a VH domain CDR3, a VL domain CDR1 and a VL domain CDR3 as defined above; an antibody, or an antigen-binding fragment thereof, comprising a VH domain CDR3, a VL domain CDR2 and a VL domain CDR3 as defined above; and an antibody, or an antigen-binding fragment thereof, comprising a VL domain CDR1, a VL domain CDR2 and a VL domain CDR3 as defined above.

In an embodiment, the antibody, or the antigen-binding fragment thereof, comprises at least four of the above described VH domain and/or VL domain CDR regions. Such embodiments include an antibody, or an antigen-binding fragment thereof, comprising a VH domain CDR1, a VH domain CDR2, a VH domain CDR3 and a VL domain CDR1 as defined above; an antibody, or an antigen-binding fragment thereof, comprising a VH domain CDR1, a VH domain CDR2, a VH domain CDR3 and a VL domain CDR2 as defined above; an antibody, or an antigen-binding fragment thereof, comprising a VH domain CDR1, a VH domain CDR2, a VH domain CDR3 and a VL domain CDR3 as defined above; an antibody, or an antigen-binding fragment thereof, comprising a VH domain CDR1, a VH domain CDR2, a VL domain CDR1 and a VL domain CDR2 as defined above; an antibody, or an antigen-binding fragment thereof, comprising a VH domain CDR1, a VH domain CDR2, a VL domain CDR1 and a VL domain CDR3 as defined above; an antibody, or an antigen-binding fragment thereof, comprising a VH domain CDR1, a VH domain CDR2, a VL domain CDR2 and a VL domain CDR3 as defined above; an antibody, or an antigen-binding fragment thereof, comprising a VH domain CDR1, a VH domain CDR3, a VL domain CDR1 and a VL domain CDR2 as defined above; an antibody, or an antigen-binding fragment thereof, comprising a VH domain CDR1, a VH domain CDR3, a VL domain CDR1 and a VL domain CDR3 as defined above; an antibody, or an antigen-binding fragment thereof, comprising a VH domain CDR1, a VH domain CDR3, a VL domain CDR2 and a VL domain CDR3 as defined above; an antibody, or an antigen-binding fragment thereof, comprising a VH domain CDR1, a VL domain CDR1, a VL domain CDR2 and a VL domain CDR3 as defined above; an antibody, or an antigen-binding fragment thereof, comprising a VH domain CDR2, a VH domain CDR3, a VL domain CDR1 and a VL domain CDR2 as defined above; an antibody, or an antigen-binding fragment thereof, comprising a VH domain CDR2, a VH domain CDR3, a VL domain CDR1 and a VL domain CDR3 as defined above; an antibody, or an antigen-binding fragment thereof, comprising a VH domain CDR2, a VH domain CDR3, a VL domain CDR2 and a VL domain CDR3 as defined above; an antibody, or an antigen-binding fragment thereof, comprising a VH domain CDR2, a VL domain CDR1, a VL domain CDR2 and a VL domain CDR3 as defined above; and an antibody, or an antigen-binding fragment thereof, comprising a VH domain CDR3, a VL domain CDR1, a VL domain CDR2 and a VL domain CDR3 as defined above.

In an embodiment, the antibody, or the antigen-binding fragment thereof, comprises at least five of the above described VH domain and/or VL domain CDR regions. Such embodiments include an antibody, or an antigen-binding fragment thereof, comprising a VH domain CDR1, a VH domain CDR2, a VH domain CDR3, a VL domain CDR1 and a VL domain CDR2 as defined above; an antibody, or an antigen-binding fragment thereof, comprising a VH domain CDR1, a VH domain CDR2, a VH domain CDR3, a VL domain CDR1 and a VL domain CDR3 as defined above; an antibody, or an antigen-binding fragment thereof, comprising a VH domain CDR1, a VH domain CDR2, a VH domain CDR3, a VL domain CDR2 and a VL domain CDR3 as defined above; an antibody, or an antigen-binding fragment thereof, comprising a VH domain CDR1, a VH domain CDR2, a VL domain CDR1, a VL domain CDR2 and a VL domain CDR3 as defined above; an antibody, or an antigen-binding fragment thereof, comprising a VH domain CDR1, a VH domain CDR3, a VL domain CDR1, a VL domain CDR2 and a VL domain CDR3 as defined above; and an antibody, or an antigen-binding fragment thereof, comprising a VH domain CDR2, a VH domain CDR3, a VL domain CDR1, a VL domain CDR2 and a VL domain CDR3 as defined above.

In an embodiment, the antibody, or the antigen-binding fragment thereof, comprises all six of the above described VH domain and VL domain CDR regions. Such embodiments include an antibody, or an antigen-binding fragment thereof, comprising a VH domain CDR1, a VH domain CDR2, a VH domain CDR3, a VL domain CDR1, a VL domain CDR2 and a VL domain CDR 3 as defined above.

In an embodiment, the antibody, or the antigen-binding fragment thereof, comprises a VH domain comprising, preferably consisting of, the amino acid sequence

(SEQ ID NO: 13)
EVQLLESGGGLVQPGGSLRLSCAASGFTFSGSYMSWRQAPGKGLEWVSS
IYGSGGYTYYADSVKGRFTISRDNSKNTLYLQMNSLRAEDTAVYYCARY
SPFYMDYWGQGTLVTVSS.

In an embodiment, the antibody, or the antigen-binding fragment thereof, comprises a VL domain comprising, preferably consisting of, the amino acid sequence

(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQGYSFPPTF
GQGTKLEIK.

In a particular embodiment, the antibody, or the antigen-binding fragment thereof, comprises a VH domain comprising, preferably consisting of, the amino acid sequence of SEQ ID NO: 13 and a VL domain comprising, preferably consisting of, the amino acid sequence of SEQ ID NO: 14.

A particularly preferred antibody of the embodiment is denoted ME107-117 or W-ME107-117 herein and the corresponding antigen-binding fragment thereof is denoted ME107-117 scFv or W-ME107-117 scFv. This antibody and antigen-binding fragment thereof binds specifically to the epitopes in the beta sheet area of IL13Rα2 (FIG. 10) and comprises VH and VL domain CDR regions as presented above.

Another aspect of the embodiments relates to an antibody, or an antigen-binding fragment thereof, capable of binding to IL13Rα2. The antibody, or the antigen-binding fragment thereof, comprises a VH domain CDR1 comprising, preferably consisting of, the amino acid sequence GFTFX₁X₂X₃X₄, wherein each Xₙ, n=1 . . . 4, is independently selected from the group consisting of G, A, S and Y. The antibody, or the antigen-binding fragment thereof, also comprises a VH domain CDR2 comprising, preferably consisting of, the amino acid sequence IB₁B₂B₃B₄B₅B₆T, wherein each Bₘ, m=1 . . . 6, is independently selected from the group consisting of G, S and Y. The antibody, or the antigen-binding fragment thereof, further comprises a VH domain CDR3 comprising, preferably consisting of, the amino acid sequence AR-Z_{H}-Z₁DY, wherein Z₁ is selected from the group consisting of F, M, I and L and Z_{H} represents an amino acid sequence selected from the group consisting of WRSTYGY (SEQ ID NO: 15), YGHYAYGSY (SEQ ID NO: 16), YSSSGWYYGF (SEQ ID NO: 17), TPYSAY (SEQ ID NO: 18), RYRSHRPGLS (SEQ ID NO: 19), FHPRYGY (SEQ ID NO: 20), GSYSHYGAHY (SEQ ID NO: 21), YYHYDYGYYY (SEQ ID NO: 22), YSPFY (SEQ ID NO: 3), RNYWEHGGGS (SEQ ID NO: 24), HHYGYYPPGSVYY (SEQ ID NO: 25), and VEYTYYGSEGSPV (SEQ ID NO: 26). The antibody, or the antigen-binding fragment thereof, comprises a VL domain CDR1 comprising, preferably consisting of, the amino acid sequence QSISSY (SEQ ID NO: 12). The antibody, or the antigen-binding fragment thereof, also comprises a VL domain CDR2 comprising, preferably consisting of, the amino acid sequence AAS and a VL domain CDR3 comprising, preferably consisting of, the amino acid sequence QQ-Z_{L}-T, wherein Z_{L} represents an amino acid sequence selected from the group consisting of TYYSPH (SEQ ID NO: 28), DYYLF (SEQ ID NO: 29), SYSTPY (SEQ ID NO: 30), FYSYPL (SEQ ID NO: 31), AFSPS (SEQ ID NO: 32), SYDTLL (SEQ ID NO: 33), ALSSLP (SEQ ID NO: 34), FSTRLS (SEQ ID NO: 35), GYSFPP (SEQ ID NO: 4), STYPF (SEQ ID NO: 37), YGSNPL (SEQ ID NO: 38), and RYNGLF (SEQ ID NO: 39).

In an embodiment, the antibody, or the antigen-binding fragment thereof, comprises an extended VH CDR1 comprising, preferably consisting of, the amino acid sequence GFTFX₁X₂X₃X₄MX₅. In this embodiment, each Xₙ, n=1 . . . 5, is independently selected from the group consisting of G, A, S and Y.

In an embodiment, X₅ is S or G.

In an embodiment, the antibody, or the antigen-binding fragment thereof, comprises an extended VH CDR2 comprising, preferably consisting of, the amino acid sequence JIB₁B₂B₃B₄B₅B₆TY. In this embodiment, each Bₘ, m=1 . . . 6, is independently selected from the group consisting of G, S and Y and J is selected from the group consisting of A, Y, G and S.

In an embodiment, J is selected from the group consisting of A, Y and S.

In an embodiment, X₁ is S or Y; X₂ is S or G; X₃ is S or Y; and X₄ is A, Y or G.

In an embodiment, B₁ is S or Y; B₂ is G; B₃ is S, G or Y; B₄ is G; B₅ is S or G; and B₆ is S or Y.

In an embodiment, Z_{H}-Z₁ represents an amino acid sequence selected from the group consisting of YGHYAYGSYF (SEQ ID NO: 40), TPYSAYI (SEQ ID NO: 41), GSYSHYGAHYL (SEQ ID NO: 42), and YSPFYM (SEQ ID NO: 9).

In an embodiment, Z_{L} represents an amino acid sequence selected from the group consisting of DYYLF (SEQ ID NO: 29), FYSYPL (SEQ ID NO: 31), ALSSLP (SEQ ID NO: 34), and GYSFPP (SEQ ID NO: 4).

Currently preferred antigen-binding fragments of the embodiments are denoted W-ME107-7, W-ME107-10, W-ME107-16, W-ME107-27, W-ME107-55, W-ME107-67, W-ME107-75, W-ME107-112, W-ME107-117, W-ME107-128, W-ME107-150 and W-ME107-156 herein. Correspondingly preferred antibodies have CDR regions and VH and VL domains corresponding to these antigen-binding fragments. Table 1 shows the VH domain CDR1, Table 2 shows the VH domain extended CDR1, Table 3 shows the VH domain CDR2 with Table 4 shows VH domain extended CDR2. Table 5 shows the VH domain CDR3 and Table 6 shows the VL domain CDR3. All of W-ME107-7, W-ME107-10, W-ME107-16, W-ME107-27, W-ME107-55, W-ME107-67, W-ME107-75, W-ME107-112, W-ME107-117, W-ME107-128, W-ME107-150 and W-ME107-156 have a common VL domain CDR1 of QSISSY (SEQ ID NO: 12) and a common VL domain CDR2 of AAS.

TABLE l
VH domain CDR1

| Clone | VH domain CDR1 | SEQ ID NO: |
|---|---|---|
| W-ME107-7 | GFTF GYYY | 100 |
| W-ME107-10 | GFTF SSYA | 101 |
| W-ME107-16 | GFTF SSYA | 101 |
| W-ME107-27 | GFTF YGSY | 102 |
| W-ME107-55 | GFTF SSYA | 101 |
| W-ME107-67 | GFTF GSSY | 103 |
| W-ME107-75 | GFTF YSYG | 104 |
| W-ME107-112 | GFTF SSYA | 101 |
| W-ME107-117 | GFTF SGSY | 105 |
| W-ME107-128 | GFTF YSYG | 104 |
| W-ME107-150 | GFTF SSYA | 101 |
| W-ME107-156 | GFTF SSYG | 106 |

TABLE 2
VH domain extended CDR1

| Clone | VH domain extended CDR1 | SEQ ID NO: |
|---|---|---|
| W-ME107-7 | GFTF GYYY M Y | 47 |
| W-ME107-10 | GFTF SSYA M S | 48 |
| W-ME107-16 | GFTF SSYA M S | 48 |
| W-ME107-27 | GFTF YGSY M G | 49 |
| W-ME107-55 | GFTF SSYA M S | 48 |
| W-ME107-67 | GFTF GSSY M Y | 50 |
| W-ME107-75 | GFTF YSYG M S | 51 |
| W-ME107-112 | GFTF SSYA M S | 48 |
| W-ME107-117 | GFTF SGSY M S | 6 |
| W-ME107-128 | GFTF YSYG M S | 51 |
| W-ME107-150 | GFTF SSYA M S | 48 |
| W-ME107-156 | GFTF SSYG M S | 52 |

TABLE 3
VH domain CDR2

| Clone | VH domain CDR2 | SEQ ID NO: |
|---|---|---|
| W-ME107-7 | I SGSGGS T | 53 |
| W-ME107-10 | I SGSGGS T | 53 |
| W-ME107-16 | I SGGGSY T | 54 |
| W-ME107-27 | I SGYGGY T | 55 |
| W-ME107-55 | I SGSGGS T | 53 |
| W-ME107-67 | I SGSGSY T | 56 |
| W-ME107-75 | I SGGGSY T | 54 |
| W-ME107-112 | I SGSGGS T | 53 |
| W-ME107-117 | I YGSGGY T | 7 |
| W-ME107-128 | I SSGSSY T | 57 |
| W-ME107-150 | I SGSGGS T | 53 |
| W-ME107-156 | I SGGGSY T | 54 |

TABLE 4
VH domain extended CDR2

| Clone | VH domain extended CDR2 | SEQ ID NO: |
|---|---|---|
| W-ME107-7 | A I SGSGGS T Y | 58 |
| W-ME107-10 | A I SGSGGS T Y | 58 |
| W-ME107-16 | Y I SGGGSY T Y | 59 |
| W-ME107-27 | Y I SGYGGY T Y | 60 |
| W-ME107-55 | A I SGSGGS T Y | 58 |
| W-ME107-67 | G I SGSGSY T Y | 61 |
| W-ME107-75 | Y I SGGGSY T Y | 62 |
| W-ME107-112 | A I SGSGGS T Y | 58 |
| W-ME107-117 | S I YGSGGY T Y | 8 |
| W-ME107-128 | S I SSGSSY T Y | 63 |
| W-ME107-150 | A I SGSGGS T Y | 58 |
| W-ME107-156 | Y I SGGGSY T Y | 59 |

TABLE 5
VH domain CDR3

| Clone | VH domain CDR3 | SEQ ID NO: |
|---|---|---|
| W-ME107-7 | AR WRSTYGY F DY | 64 |
| W-ME107-10 | AR YGHYAYGSY F DY | 65 |
| W-ME107-16 | AR YSSSGWYYGF M DY | 66 |
| W-ME107-27 | AR TPYSAY I DY | 67 |
| W-ME107-55 | AR RYRSHRPGLS F DY | 68 |
| W-ME107-67 | AR FHPRYGY F DY | 69 |
| W-ME107-75 | AR GSYSHYGAHY L DY | 70 |
| W-ME107-112 | AR YYHYDYGYYY F DY | 71 |
| W-ME107-117 | AR YSPFY M DY | 10 |
| W-ME107-128 | AR RNYWEHGGGS L DY | 72 |
| W-ME107-150 | AR HHYGYYPPGSVYY F DY | 73 |
| W-ME107-156 | AR VEYTYYGSEGSPV F DY | 74 |

TABLE 6
VL domain CDR3

| Clone | VL domain CDR3 | SEQ ID NO: |
|---|---|---|
| W-ME107-7 | QQ TYYSPH T | 75 |
| W-ME107-10 | QQ DYYLF T | 76 |
| W-ME107-16 | QQ SYSTPY T | 77 |
| W-ME107-27 | QQ FYSYPL T | 78 |
| W-ME107-55 | QQ AFSPS T | 79 |
| W-ME107-67 | QQ SYDTLL T | 80 |
| W-ME107-75 | QQ ALSSLP T | 81 |
| W-ME107-112 | QQ FSTRLS T | 82 |
| W-ME107-117 | QQ GYSFPP T | 11 |
| W-ME107-128 | QQ STYPF T | 83 |
| W-ME107-150 | QQ YGSNPL T | 84 |
| W-ME107-156 | QQ RYNGLF T | 85 |

Particularly preferred antigen-binding fragments of the embodiments are W-ME107-10, W-ME107-27, W-ME107-75 and W-ME107-117, and particularly preferred antibodies are antibodies having the VH and VL CDR regions of W-ME107-10, W-ME107-27, W-ME107-75 and W-ME107-117.

In an embodiment, the antibody, or the antigen-binding fragment thereof, comprises the VH CDR regions of clone W-ME107-10 as specified in Tables 1, 3 and 5, preferably as specified in Tables 2, 4 and 5.

In an embodiment, the antibody, or the antigen-binding fragment thereof, comprises the VL CDR3 regions of clone W-ME107-10 as specified in Table 6 together with the common VL domain CDR1 and CDR2 regions.

In a particular embodiment, the antibody, or the antigen-binding fragment thereof, comprises the VH CDR regions of clone W-ME107-10 as specified in Tables 1, 3 and 5, preferably as specified in Tables 2, 4 and 5, and the VL CDR3 regions of clone W-ME107-10 as specified in Table 6 together with the common VL domain CDR1 and CDR2 regions.

In an embodiment, the antibody, or the antigen-binding fragment thereof, comprises the VH CDR regions of clone W-ME107-27 as specified in Tables 1, 3 and 5, preferably as specified in Tables 2, 4 and 5.

In an embodiment, the antibody, or the antigen-binding fragment thereof, comprises the VL CDR3 regions of clone W-ME107-27 as specified in Table 6 together with the common VL domain CDR1 and CDR2 regions.

In a particular embodiment, the antibody, or the antigen-binding fragment thereof, comprises the VH CDR regions of clone W-ME107-27 as specified in Tables 1, 3 and 5, preferably as specified in Tables 2, 4 and 5, and the VL CDR3 regions of clone W-ME107-27 as specified in Table 6 together with the common VL domain CDR1 and CDR2 regions.

In an embodiment, the antibody, or the antigen-binding fragment thereof, comprises the VH CDR regions of clone W-ME107-75 as specified in Tables 1, 3 and 5, preferably as specified in Tables 2, 4 and 5.

In an embodiment, the antibody, or the antigen-binding fragment thereof, comprises the VL CDR3 regions of clone W-ME107-75 as specified in Table 6 together with the common VL domain CDR1 and CDR2 regions.

In a particular embodiment, the antibody, or the antigen-binding fragment thereof, comprises the VH CDR regions of clone W-ME107-75 as specified in Tables 1, 3 and 5, preferably as specified in Tables 2, 4 and 5, and the VL CDR3 regions of clone W-ME107-75 as specified in Table 6 together with the common VL domain CDR1 and CDR2 regions.

In an embodiment, the antibody, or the antigen-binding fragment thereof, comprises the VH CDR regions of clone W-ME107-117 as specified in Tables 1, 3 and 5, preferably as specified in Tables 2, 4 and 5.

In an embodiment, the antibody, or the antigen-binding fragment thereof, comprises the VL CDR3 regions of clone W-ME107-117 as specified in Table 6 together with the common VL domain CDR1 and CDR2 regions.

In a particular embodiment, the antibody, or the antigen-binding fragment thereof, comprises the VH CDR regions of clone W-ME107-117 as specified in Tables 1, 3 and 5, preferably as specified in Tables 2, 4 and 5, and the VL CDR3 regions of clone W-ME107-117 as specified in Table 6 together with the common VL domain CDR1 and CDR2 regions.

W-ME107-10 comprises a VH domain of (SEQ ID NO: 86):
EVQLLESGGGLVQPGGSLRLSCAASGFTFSSYAMSWVRQAPGKGLEWVSAISGSGGSTYYADSVKG
RFTISRDNSKNTLYLQMNSLRAEDTAVYYCARYGHYAYGSYFDYWGQGTLVTVSS
and
a VL domain of (SEQ ID NO: 87):
DIQMTQSPSSLSASVGDRVTITCRASQSISSYLNWYQQKPGKAPKLLIYAASSLQSGVPSRFSGSGSGT
DFTLTISSLQPEDFATYYCQQDYYLFTFGQGTKLEIK
W-ME107-27 comprises a VH domain of (SEQ ID NO: 88):
EVQLLESGGGLVQPGGSLRLSCAASGFTFYGSYMGWVRQAPGKGLEWVSYISGYGGYTYYADSVKG
RFTISRDNSKNTLYLQMNSLRAEDTAVYYCARTPYSAYIDYWGQGTLVTVSS
and
a VL domain of (SEQ ID NO: 89):
DIQMTQSPSSLSASVGDRVTITCRASQSISSYLNWYQQKPGKAPKLLIYAASSLQSGVPSRFSGSGSGT
DFTLTISSLQPEDFATYYCQQFYSYPLTFGQGTKLEIK
W-ME107-75 comprises a VH domain of (SEQ ID NO: 90):
EVQLLESGGGLVQPGGSLRLSCAASGFTFYSYGMSWVRQAPGKGLEWVSYISGGGSYTYYADSVKG
RFTISRDNSKNTLYLQMNSLRAEDTAVYYCARGSYSHYGAHYLDYWGQGTLVTVSS
and
a VL domain of (SEQ ID NO: 91):
DIQMTQSPSSLSASVGDRVTITCRASQSISSYLNWYQQKPGKAPKLLIYAASSLQSGVPSRFSGSGSGT
DFTLTISSLQPEDFATYYCQQALSSLPTFGQGTKLEIK
W-ME107-117 comprises a VH domain of (SEQ ID NO: 13):
EVQLLESGGGLVQPGGSLRLSCAASGFTFSGSYMSWVRQAPGKGLEWVSSIYGSGGYTYYADSVKG
RFTISRDNSKNTLYLQMNSLRAEDTAVYYCARYSPFYMDYWGQGTLVTVSS
and
a VL domain of (SEQ ID NO: 14):
DIQMTQSPSSLSASVGDRVTITCRASQSISSYLNWYQQKPGKAPKLLIYAASSLQSGVPSRFSGSGSGT
DFTLTISSLQPEDFATYYCQQGYSFPPTFGQGTKLEIK

In an embodiment, the VH domain of the antibody, or the antigen-binding fragment thereof, is fused to the VL domain through a linker. Various such linkers commonly used to interconnect VH and VL domains in antibodies and antigen-binding fragments thereof could be used according to the embodiments. In a particular embodiment, the linker is a peptide linker. For instance, the peptide linker could comprise, such as consist of, the amino acids glycine (G) and/or serine (S). An illustrative, but non-limiting, example of such a peptide linker comprises, preferably consists of, the amino acid sequence of GGGGSGGGGSGGGGS (SEQ ID NO: 27).

The skilled person will appreciate that minor variations, such as substitutions, including deletion or addition of amino acids, of one, two, three, four or even more amino acid residues, in the amino acid sequences may occur without effecting the functional properties, such as its ability to bind to IL13Rα2, of the antibody, or antigen-binding fragment thereof. The variation may be in the amino acid sequence of the CDRs, in the amino acid sequence outside the CDR regions, i.e., the framework regions, or both in the amino acid sequence of the CDRs and in the amino acid sequence outside the CDR regions of the heavy and/or light chain variable regions. Accordingly, the embodiments also encompass antibodies, or antigen-binding fragments thereof, having a sequence identity of at least 75%, preferably at least 80%, such as at least 85%, and more preferably at least 86%, at least 87%, at least 88%, at least 89%, at least 90%, at least 91%, at least 92%, at least 93%, at least 94%, at least 95%, at least 96%, at least 97%, at least 98% or at least 99% of any of the amino acid sequence presented herein or in the sequence listing.

As used herein, sequence identity refers to sequence similarity between two amino acid sequences, such as peptide or protein sequences. The similarity is determined by sequence alignment to determine the structural and/or functional relationships between the sequences. Sequence identity between amino acid sequences can be determined by comparing an alignment of the sequences using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings (for protein alignment, Gap costs Existence:11 Extension:1). Sequence comparisons and percentage identities mentioned in this specification have been determined using this software. When comparing the level of sequence identity to, for example, an amino acid sequence this, preferably should be done relative to the whole length of the amino acid sequence, i.e., a global alignment method is used to avoid short regions of high identity overlap resulting in a high overall assessment of identity. For example, a short polypeptide fragment having, for example, five amino acids might have a 100% identical sequence to a five amino acid region within the whole of the amino acid sequence, but this does not provide a 100% amino acid identity unless the fragment forms part of a longer sequence which also has identical amino acids at other positions equivalent to positions in the amino acid sequence. When an equivalent position in the compared sequences is occupied by the same amino acid, then the molecules are identical at that position. Scoring an alignment as a percentage of identity is a function of the number of identical amino acids at positions shared by the compared sequences. When comparing sequences, optimal alignments may require gaps to be introduced into one or more of the sequences, to take into consideration possible insertions and deletions in the sequences.

In an embodiment, the antibody is a monoclonal antibody. In another embodiment, the antibody is a polyclonal antibody.

In an embodiment, the antibody is a genetically engineered antibody, e.g., a single-chain antibody, a humanized antibody, a chimeric antibody, a CDR-grafted antibody, a humanized antibody, a bispecific antibody, or a multi-specific antibody. In an embodiment, the antibody is a chimeric antibody. Chimeric antibody as used herein refers to an antibody comprising constant domains from one species and the variable domains from a second species.

In an embodiment, the antibody is a humanized antibody. Humanized as used herein refers to antibodies having at least one CDR region from a non-human source that are engineered to have a structure and immunological function more similar to true human antibodies than the original source antibodies. An example of a humanized antibody is an antibody having CDR regions from a non-human antibody grafted into a human antibody. Humanizing can additionally, or alternatively, involve selected amino acid substitutions to make a non-human amino acid sequence be more like a human sequence.

Suitable methods of making antibodies are known in the art. For instance, standard hybridoma methods are described in Harlow and Lane (eds.), Antibodies: A Laboratory Manual, CSH Press (1988), and CA. Janeway et al. (eds.), Immunobiology, 5th Ed., Garland Publishing, New York, N.Y. (2001)).

Monoclonal antibodies may be prepared using any technique that provides for the production of antibody molecules by continuous cell lines in culture. These include, but are not limited to, the hybridoma technique originally described in Nature 256: 495-497, 1975, the human B-cell hybridoma technique (Immunol Today 4:72, 1983; Proc Natl Acad Sci 80: 2026-2030, 1983) and the EBV-hybridoma technique (Cole et al., Monoclonal Antibodies and Cancer Therapy, Alan R Liss Inc, New York N.Y., pp 77-96, (1985)).

Polyclonal antibodies may be prepared by immunizing an animal with an immunogen comprising the IL13Rα2 antigen and collecting antisera from that immunized animal. A wide range of animal species can be used for the production of antisera including, but not-limited to, rabbits, mice, rats, hamsters, goats, sheep, pigs or horses.

Antibodies, and antigen-binding fragments, thereof may alternatively be produced using phage display selection as described herein.

An antigen-binding fragment of an antibody as used herein can be selected from a group consisting of a single-chain antibody, an Fv fragment, an scFv fragment, an Fab fragment, an F(ab′)2 fragment, an Fab′ fragment, an Fd fragment, a single-domain antibody (sdAb), an scFv-Fc fragment, a di-scFv fragment and a CDR region. A currently preferred embodiment of antigen-binding fragment is a single-chain variable fragment (scFv).

In an embodiment, the antibody, or the antigen-binding fragment thereof, binds specifically to IL13Rα2, preferably human IL13Rα2. In an embodiment, the antibody, or the antigen-binding fragment thereof, additionally, or alternatively, binds specifically to a non-human IL13Rα2. Illustrative, but non-limiting, examples of such non-human IL13Rα2 include canine IL13Rα2, feline IL13Rα2, bovine IL13Rα2, equine IL13Rα2, ovine IL13Rα2, rat IL13Rα2 and/or mouse IL13Rα2.

In an embodiment, the antibody, or the antigen-binding fragment thereof, does not bind to human IL13Rα1.

An aspect of the embodiments relates to a chimeric antigen receptor (CAR). The CAR comprises an antigen recognition domain comprising an antibody, or an antigen-binding fragment thereof, according to the embodiments. The CAR also comprises a transmembrane domain and an intracellular signaling domain.

In general, CARs comprise an ectodomain, a transmembrane domain, and an endodomain. The ectodomain of a CAR comprises an antigen recognition region, which may be a scFv. The ectodomain may also comprise a signal tag or peptide that directs the CAR into the endoplasmic reticulum.

The transmembrane domain is the portion of the CAR that traverses the cell membrane. In general, the transmembrane domain can be derived from any transmembrane protein. In an embodiment, the transmembrane domain comprises a hydrophobic alpha helix. Illustrative, but non-limiting, examples of transmembrane domains that can be included in the CAR include all, or a portion, of the transmembrane domain of cluster of differentiation 28 (CD28), all, or a portion, of the transmembrane domain of CD8a, all, or a portion, of the transmembrane domain of CD27, all, or a portion, of the transmembrane domain of CD137 (4-1BB), all, or a portion, of the transmembrane domain of CD134 (OX40), all, or a portion, of the transmembrane domain of CD3ε, all, or a portion, of the transmembrane domain of CD3ζ, all, or a portion, of the transmembrane domain of CD3γ, all, or a portion, of the transmembrane domain of CD3δ, all, or a portion, of the transmembrane domain of TCRα, and all, or a portion, of the transmembrane domain of TCRβ, preferably all, or a portion, of the transmembrane domain of CD28 or all, or a portion, of the transmembrane domain of CD8α.

The endodomain of a CAR comprises at least one signaling domain. Illustrative, but non-limiting, examples of such signaling domains that can be included in the CAR include the zeta chain of CD3 (CD3ζ), CD28, CD137 (4-1BB), ICOS, CD27, CD40, OX40 (CD134), or Myd88, preferably CD3ζ and/or CD137.

In an embodiment, the CAR comprises a hinge domain or spacer interconnecting the antigen recognition domain and the transmembrane domain.

A related aspect of the embodiments defines a T cell receptor (TCR) complex comprising an antigen recognition domain comprising an antibody, or an antigen-binding fragment thereof, according to the embodiments.

A TCR is a molecule found on the surface of T cells that is responsible for recognizing fragments of antigen as peptides bound to major histocompatibility complex (MHC) molecules. When the TCR engages with an antigenic peptide and MHC, the T cell is activated through signal transduction, that is, a series of biochemical events mediated by associated enzymes, co-receptors, specialized adaptor molecules, and activated or released transcription factors. A TCR-complex is usually a TCR molecule associated with a CD3γ chain, a CD3δ chain, and two CD3ε chains. These chains associate with the TCR and the ζ-chain (zeta-chain) to generate an activation signal in T cells. The TCR, ζ-chain, and CD3 molecules together constitute the TCR-complex.

Non-limiting examples of TCRs that can be used include CMVpp65 TCR and TARP TCR.

Another aspect of the embodiments relates to a conjugate comprising an antibody, or an antigen-binding fragment thereof, according to the embodiments and an effector molecule.

A conjugate of the embodiments comprises an antibody, or an antigen-binding fragment thereof, as targeting domain or molecule and an effector domain or molecule. The antibody, or an antigen-binding fragment thereof, targets the conjugate to cells expressing IL13Rα2, including tumor cells.

The antibody, or the antigen-binding fragment thereof, of the embodiments may be used in connection with various so-called effector domains or molecules. In an embodiment, the effector molecule is selected from the group consisting of a detectable label, a cytotoxin, a metal, another antibody or an antigen-binding fragment thereof, a nucleic acid sequence, and a lipid bilayer docking moiety

In an embodiment, the effector domain may be used for detection and diagnosis purposes. In such a case, the effector domain is a detectable label, such as a radiolabel, a fluorescent label, a chemoluminescence label. The conjugate can then be used for diagnosis and imaging of IL13Rα2, such as IL13Rα2 expressing cells, for instance in a subject body. Various imaging modalities may then be used depending on the particular detectable label, for instance, X-ray imaging, magnetic resonance imaging (MRI), positron emission tomography (PET), or single photon emission computed tomography (SPECT).

Instead of using a detectable label, the antibody, or the antigen-binding fragment thereof, of the embodiments could be detected by substituting an element, such as hydrogen or carbon, in at least one amino acid residue of the antibody, or the antigen-binding fragment thereof, with its isotope, such as deuterium instead of hydrogen and ¹³C instead of ¹²C.

Metal can be also be used as effector domain for imaging purposes including paramagnetic metals and radioisotopes.

Cytotoxin, or cytotoxic agent, can be used as effector domain to exert cytotoxicity and kill an IL13Rα2 expressing cell once the conjugate has targeted the cell using the antibody, or the antigen-binding fragment thereof. A typical example of cytotoxin is a chemotherapeutic agent. Any chemotherapeutic agent can be used according to the embodiments including, but not limited to, alkylating agents, antimetabolites, anti-microtubule agents, topoisomerase inhibitors, and cytotoxic antibiotics.

In another embodiment, the effector domain is an apoptosis tag, which causes induction of apoptosis by a cell expressing IL13Rα2 and targeted by the conjugate. An example of such an apoptosis tag is the TRAIL protein.

In yet another embodiment, the effector domain is a T cell or B cell epitope, or a nucleic acid sequence coding for the T cell or B cell epitope, which causes specific induction of T- or B-cell immunity against the epitope.

The effector domain may be another antibody, or an antigen-binding fragment thereof. For instance, the effector domain may be an Fc domain of IgG or other immunoglobulin. The Fc domain can then be used to enable purification via a Protein A affinity column. In addition, or alternatively, the Fc domain may improve the half-life of the conjugate in vivo. Furthermore, the Fc region allows for dimerization/multimerization of the conjugate.

A further aspect of the embodiments includes a nucleic acid molecule encoding an antibody, or an antigen-binding fragment thereof, a CAR and/or a TCR-complex according to the embodiments. Nucleic acid molecule as used herein includes polynucleotide, oligonucleotide, and nucleic acid sequence, and generally means a polymer of DNA or RNA, which may be single-stranded or double-stranded, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. Nucleic acid molecule also include complementary DNA (cDNA) and messenger RNA (mRNA).

The nucleic acid molecule may also encode other molecules than the antibody, or the antigen-binding fragment thereof, according to the embodiments. An illustrative example of such another molecule is Helicobacter pylori (HP) neutrophil activating protein (NAP), HP-NAP is a dodecameric protein that acts as a virulence factor in H. pylori bacterial infection. It is made of 12 monomeric subunits and each subunit is comprised of four alpha-helices. The surface of HP-NAP is highly positively charged and has capacity of interacting with and activating human white blood cells (WBCs), also denoted leukocytes.

Another aspect of the embodiments relates to a vector comprising a nucleic acid molecule according to the embodiments.

The vector is preferably an expression vector, i.e., a vector comprising at least one nucleic acid molecule comprising coding sequences that can be expressed, such as transcribed and translated, in a host cell, such as host T cell, comprising the expression vector. The expression vector is in an embodiment selected among DNA molecules, RNA molecules, plasmids, episomal plasmids and virus vectors.

In an embodiment, the vector is a virus vector. In a particular embodiment, the virus vector is selected from a group consisting of a lentiviral vector, an adenoviral vector, an adeno-associated viral vector, a retroviral vector, a Semliki Forest virus, a polio virus and a hybrid vector.

Lentiviruses are a subclass of retroviruses. They are adapted as gene delivery vehicles (vectors) thanks to their ability to integrate into the genome of non-dividing cells, which is the unique feature of lentiviruses as other retroviruses can infect only dividing cells. The viral genome in the form of RNA is reverse-transcribed when the virus enters a cell, such as a T cell, to produce DNA, which is then inserted into the genome at a position by the viral integrase enzyme. The vector, now called a provirus, remains in the genome and is passed on to the progeny of the cell if it divides. For safety reasons lentiviral vectors typically never carry the genes required for their replication. To produce a lentivirus, several plasmids are transfected into a so-called packaging cell line, commonly HEK 293. One or more plasmids, generally referred to as packaging plasmids, encode the virion proteins, such as the capsid and the reverse transcriptase. Another plasmid contains the genetic material to be delivered by the vector. It is transcribed to produce the single-stranded RNA viral genome and is marked by the presence of the ψ (psi) sequence. This sequence is used to package the genome into the virion.

Retroviruses are one of the mainstays of current gene therapy approaches. The recombinant retroviruses, such as the Moloney murine leukemia virus, have the ability to integrate into the host genome in a stable fashion. They contain a reverse transcriptase that allows integration into the host genome. Retroviral vectors can either be replication-competent or replication-defective. Replication-defective vectors are the most common choice because the viruses have had the coding regions for the genes necessary for additional rounds of virion replication and packaging replaced with other genes, or deleted. These viruses are capable of infecting T cells and delivering their viral payload, but then fail to continue the typical lytic pathway that leads to cell lysis and death. If the vector is a lentiviral or retroviral vector then the nucleic acid sequence(s) encoding CAR and/or TCR-complex and HP-NAP and/or the immunological equivalent fragment of HP-NAP is/are preferably RNA sequence(s).

Adenoviral vector as used herein include adenovirus vectors and adenovirus-derived virus vectors.

Adenoviral DNA does not integrate into the genome and is not replicated during cell division. An adeno-derived virus vector is based on an adenovirus but in which various modifications have been done, such as relating to the nucleotide sequences coding replication proteins, regulation protein, viral surface proteins, etc.

Adeno-associated virus (AAV) is a small virus that infects humans and some other primate species. AAV can infect both dividing and non-dividing cells and may incorporate its genome into that of the host cell. Moreover, AAV mostly stays as episomal, performing long and stable expression. Whereas AAV packages a single strand of DNA and requires the process of second-strand synthesis, self-complementary adeno-associated virus (scAAV) packages both strands which anneal together to form double stranded DNA. By skipping second strand synthesis, scAAV allows for rapid expression in the cell. If the vector is an adenoviral vector then the nucleic acid sequence(s) encoding CAR and/or TCR-complex and HP-NAP and/or the immunological equivalent fragment of HP-NAP is/are preferably DNA sequence(s).

The Semliki Forest virus is a positive-stranded RNA virus with a genome of approximately 13,000 base pairs which encodes nine proteins. The 5′ two thirds of the genome encode four non-structural proteins concerned with RNA synthesis and the structural proteins are encoded in the 3′ third. Of the structural proteins, the C proteins makes up the icosahedral capsid which is enveloped by a lipid bilayer, derived from the host cell. The outermost surface of the virus is almost entirely covered by heterodimers of glycoproteins E1 and E2, arranged in interconnective trimers, which form an outer shell. Trimers are anchored in the membrane by an E2 cytoplasmic domain that associates with the nucleocapsid. Due to its broad host range and efficient replication, it has also been developed as a vector.

A hybrid vector is a vector virus that is genetically engineered to have qualities of more than one vector. For instance, a hybrid vector may be a combination of an adenovirus and a lentivirus.

In an embodiment, the nucleic acid molecule(s) encoding the antibody, or the antigen-binding fragment thereof, the CAR and/or the TCR-complex is(are) under transcriptional control of a promoter. In an embodiment, the promoter is selected from a group consisting of the human EF1α promoter, the CMV promoter and the CAG promoter, preferably the EF1α promoter.

In an embodiment, the vector comprises a nucleic acid molecule encoding a CAR and/or a TCR-complex under transcriptional control of a promoter, such as the EF1α promoter. The vector also comprises a nucleic acid molecule encoding HP-NAP, preferably with a signal peptide for secretion, and under transcriptional control of an inducible promoter, such as an inducible NFAT-IL-2 promoter.

A further aspect of the embodiments relates to a cell comprising an antibody, or an antigen-binding fragment thereof; a CAR; a TCR-complex; a nucleic acid molecule and/or a vector according to the embodiments.

The nucleic acid or vector can then be transcribed in the cell to produce the antibody, or the antigen-binding fragment thereof, the CAR and/or TCR-complex in the cell.

In an embodiment, the cell is selected from the group consisting of a T cell, a natural killer (NK) cell, a B cell, a monocyte, and a macrophage. In a particular embodiment, the cell is a T cell.

The embodiments also relate to a pharmaceutical composition comprising an antibody, or an antigen-binding fragment thereof; a CAR; a TCR-complex; a cell; a conjugate; a nucleic acid; and/or a vector according to the embodiments, and a pharmaceutically acceptable carrier.

The pharmaceutically acceptable carriers could be any pharmaceutically acceptable carrier, vehicle and/or excipient, including combinations thereof, that are is or are compatible with the other constituent(s) of the pharmaceutical composition. Non-limiting examples of such pharmaceutically acceptable carriers include injection solutions, such as saline or buffered injection solutions.

The embodiments also relate to an antibody, or an antigen-binding fragment thereof; a CAR; a TCR-complex; a conjugate wherein the effector molecule is a cytotoxin; a nucleic acid molecule; a vector; a cell and/or a pharmaceutical composition according to the embodiments for use as a medicament.

In a particular embodiment, the antibody, or the antigen-binding fragment thereof; the CAR; the TCR-complex; the conjugate wherein the effector molecule is a cytotoxin; the nucleic acid molecule; the vector; the cell and/or the pharmaceutical composition according to the embodiments can be used in treating or delaying the onset of a cancer disease characterized by expression of IL13Rα2 on the cancer cells, i.e., an IL13Rα2-expressing cancer disease.

In an embodiment, the IL13Rα2-expressing cancer disease is selected from the group consisting of glioblastoma, medulloblastoma, breast cancer, head and neck cancer, pancreatic cancer, kidney cancer, ovarian cancer, colon cancer, liver cancer, lung cancer, urothelial cancer, melanoma and Kaposi's sarcoma.

A further embodiment is directed towards a method of treating, reducing and/or preventing an IL13Rα2-expressing cancer in a patient. The method comprises administering an effective amount of an antibody, or an antigen-binding fragment thereof; a CAR; a TCR-complex; a conjugate wherein the effector molecule is a cytotoxin; a nucleic acid molecule; a vector; a cell and/or a pharmaceutical composition according to the embodiments to the patient.

As used herein, effective amount indicates an amount effective, at dosages and for periods of time necessary to achieve a desired result. For example, in the context of inhibiting a tumor growth an effective amount is an amount that, for example, induces, remission, reduces tumor burden, and/or prevents tumor spread or growth compared to the response obtained without administration of the cells. Effective amounts may vary according to factors, such as the disease state, age, sex, weight of the patient. Treating or treatment as used herein and is well understood in the art, means an approach for obtaining beneficial or desired results, including clinical results. Beneficial or desired clinical results could include, for instance, alleviation or amelioration of one or more symptoms or conditions, diminishment of extent of disease, stabilized state of disease, i.e. prevent worsening, preventing spread of disease, delay or slowing of disease progression, amelioration or palliation of the disease state, diminishment of the reoccurrence of disease, and remission. Treating or treatment may also prolong survival as compared to expected survival if not receiving any treatment.

Preventing or prophylaxis as used herein and is well understood in the art, means an approach in which a risk of developing a disease or condition is reduced or prevented, including prolonging or delaying disease development. For instance, a patient predisposed to develop a disease, such as due to genetic or hereditary predisposition, could benefit for administration of the antibody, or the antigen-binding fragment thereof, the cell, the conjugate and/or the pharmaceutical composition according to the embodiments to prevent, reduce the risk of, delaying and/or slowing development of the disease.

The patient is preferably a human patient. The embodiments may, however, also be applied in veterinary applications, i.e., non-human patients, such as non-human mammals including, for instance, primates, monkeys, apes, cattle, sheep, pigs, goats, horses, cats, dogs, mice, rats and guinea pigs. The antibody, or the antigen-binding fragment thereof, the cell, the conjugate and/or the pharmaceutical composition according to the embodiments may be administered to the patient according to various routes including, for instance, intravenous, subcutaneous, intraperitoneal, intramuscular or intratumoral administration.

The present invention also relates to a method of identifying an IL13Rα2-positive cell. The method comprises contacting a biological sample with an antibody, or an antigen-binding fragment thereof, according to embodiments and measuring the amount of the antibody, or the antigen-binding fragment thereof, bound to at least one cell of the biological sample, thereby identifying the at least one cell as an IL13Rα2-positive cell.

In an embodiment, the antibody, or the antigen-binding fragment thereof, comprises an isotope or is fused to or connected to, such as using a biotin-avidin or biotin-streptavidin link, a detectable label as previously described herein.

Alternatively, the IL13Rα2-positive cell could be detected using flow cytometry (FCM) or Enzyme-Linked Immunosorbent Assay (ELISA).

### EXAMPLES

The Examples herein describe the development of human single-chain antibody variable fragment (scFv) that targeted IL13Rα2 for use in chimeric antigen receptor (CAR) T cell therapy for treatment of cancer.

### Example 1—Conversion of 1E10B9 and mAb47 IgG to scFv and Binding Characterization by ELISA and HTRF

A literature study revealed two existing monoclonal anti-IL13Rα2, namely 1E10B9 (U.S. Pat. No. 9,868,788; Debinski, et al., New agents for targeting of IL-13RA2 expressed in primary human and canine brain tumors, PLoS One 2013, 8(10): e77719)) and mAb clone 47 (mAb47) (U.S. Pat. No. 10,308,719; Balyasnikova et al., Characterization and immunotherapeutic implications for a novel antibody targeting interleukin (IL)-13 receptor α2, J Biol Chem 2012, 287(36): 30215-30227; Kim et al., A novel single-chain antibody redirects adenovirus to IL13Rα2-expressing brain tumors, Sci Rep 2015, 5: 18133). These are both mouse antibodies generated by the hybridoma technology. In case of 1E10B9, a peptide fragment encoding part of the extracellular domain of IL13Rα2, with 100% homology between human and canine sequences, was synthesized and used as immunogen. The resulting antibody 1E10B9, thus, binds both human and canine orthologues of the receptor. mAb47 was obtained by using the full extracellular region of the receptor as immunogen (IL13Rα2-Fc). The reported affinity to human IL13Rα2 is K_{D}=1.4 nM and no binding to human IL13Rα1 or to mouse IL13Rα2 was seen (Balyasnikova et al., Characterization and immunotherapeutic implications for a novel antibody targeting interleukin (IL)-13 receptor α2, J Biol Chem 2012, 287(36): 30215-30227). Also, this antibody competes with IL-13 for binding to IL13Rα2.

Genes encoding scFv proteins of 1E10B9 and mAb47 were synthesized, protein expressed in bacteria and tested for binding to human IL13Rα2 by ELISA and homogeneous time resolved fluorescence (HTRF). The result of these assays showed that only mAb47 scFv retained the binding ability of its full-length parental IgG counterpart.

Material and Methods

Gene Synthesis

Sequences of the variable domains of the heavy (VH) and light (VL) chains of 1E10B9 and mAb47 were obtained from U.S. Pat. Nos. 9,868,788 and 10,308,719, respectively. The first seven amino acids of the VH of 1E10B9 were not included in the patent. In order to create a complete VH gene, the first seven amino acids of the most homologous mouse VH gene was included based on searches in the IMGT database (http://www.imgt.org/3Dstructure-DB/cgi/DomainGapAlign.cgi). By fusing the VH to the VL via a glycine-serine linker ((Gly4Ser)₃), genes encoding the corresponding scFv constructs 1E10B9 scFv, also referred to as X-ME107-B9, (SEQ ID NO: 92) and mAb47 scFv, also referred to as X-ME107-47, (SEQ ID NO: 93) were formed.

1E10B9 scFv (VH + linker + VL + 3xFLAG + Hisx6)*
(SEQ ID NO: 92)
QIQLVQSGPELKKPGETVKIYCKASGYSFRDYSVHWVKQAPGKGLKWMGWINTETGEPTYVD
EFKGRFAFFLEASANTVYLQISNLKNEDTATYFCDYRFTYWGQGTLVTVSAGGGGSGGGGSG
GGGSEIVMTQTPLILSVTIGQPASISCKSSQSVLYSNGKTYLNWLLQRPGQSPKRLIYLVSK
LDSGVPDRFTGSGSGTDFTLKISRVEAEDLGVYYCVQGSHFPYTFGGGTKLEIKAAADYKDH
DGDYKDHDIDYKDDDDKAAAHHHHHH
mAb47 scFv (VH + linker + VL + 3xFLAG + Hisx6)
(SEQ ID NO: 93)
QVQLQQPGAELVRPGASVKLSCKASGYTFSNYLMNWVKQRPEQDLDWIGRIDPYDGDIDYNQ
NFKDKAILTVDKSSSTAYMQLSSLTSEDSAVYYCARGYGTAYGVDYWGQGTSVTVSSGGGGS
GGGGSGGGGSDIVLTQSPASLAVSLGQRATISCRASESVDNYGISFMNWFQQKPGQPPKLLI
YAASRQGSGVPARFSGSGSGTDFSLNIHPMEEDDTAMYFCQQSKEVPWTFGGGTKLEIKAAA
DYKDHDGDYKDHDIDYKDDDDKAAAHHHHHH

Synthesis and sub-cloning of 1E10B9 scFv and mAb47 scFv were performed by GenScript (Piscataway, N.J., USA). Codon optimizations of these were performed based on Escherichia coli expression. After synthesis, the scFv genes were cloned into the pHAT-6 vector (SciLifeLab, Stockholm, Sweden) using restriction enzymes SfiI and NotI. The pHAT-6 vector provides the secreted scFv with a triple-FLAG tag and a hexahistidine (His×6) tag at the C-terminus.

The vectors were transformed into Top10 E. coli and the intended sequences confirmed by sequencing (GATC, Germany).

Protein Expression

Small-scale expression of 1E10B9 scFv and mAb47 scFv, two colonies per scFv, was carried out in 96-deep-well plates. Following overnight incubation, the bacteria were spun down and the supernatants used for functional assessment in both an ELISA and HTRF.

ELISA

Human IL13Rα2-avi (see Example A1), and negative control protein streptavidin were coated into a 384-ELISA well plate at 1 μg/ml in PBS at 4° C. overnight.

Plates were washed twice with MilliQ water and blocked for 2 h in blocking buffer (phosphate-buffered saline (PBS) supplemented with 0.5% bovine serum albumin (BSA)+0.05% TWEEN 20®). Triple-FLAG-tagged scFv 1E10B9 and mAb47, present in bacterial supernatant, were diluted 1:2, 1:20 and 1:200 in blocking buffer and allowed to bind. An assay control scFv (G-strep-1), specific for streptavidin, was also included. Detection of binding was enabled through an HRP-conjugated anti-FLAG M2 antibody (Sigma-Aldrich #A8592) followed by incubation with 1-step Ultra TMB ELISA substrate (ThermoFisher Scientific #34029). The colorimetric signal development was stopped by adding 1 M sulfuric acid and plates were read at 450 nm. All samples were assayed in duplicates.

Homogeneous Time Resolved Fluorescence (HTRF)

1E10B9, mAb47 and a positive assay control scFv were diluted 1:5 in assay buffer (PBS supplemented with 0.1% BSA) and allowed to bind to human IL13Rα2-avi, and a non-relevant protein, both diluted to 200 nM in assay buffer. Detection of binding was enabled through donor molecule terbium-conjugated anti-FLAG antibody (Cisbio #611FG2TL) and acceptor molecule streptavidin-conjugated XL665 (Cisbio #610SAXL). Plates were incubated dark for 2 h at room temperature before being analyzed on an Envision spectrometer (Perkin Elmer) at 615 nm (background/noise signal) and 665 nm (binding signal). All samples were assayed in duplicates.

Results

4 μg of vectors encoding 1E10B9 scFv and mAb47 scFv were obtained from GenScript. DNA sequencing of these verified the correct sequences. Small-scale expressions were performed and the binding of these analyzed to a small set of antigens using ELISA and HTRF.

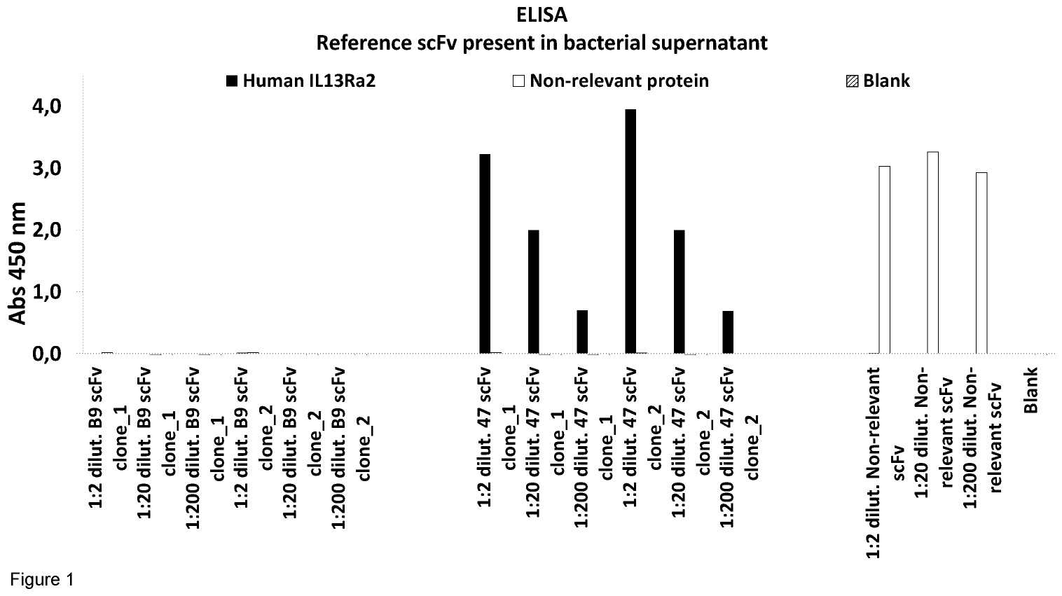

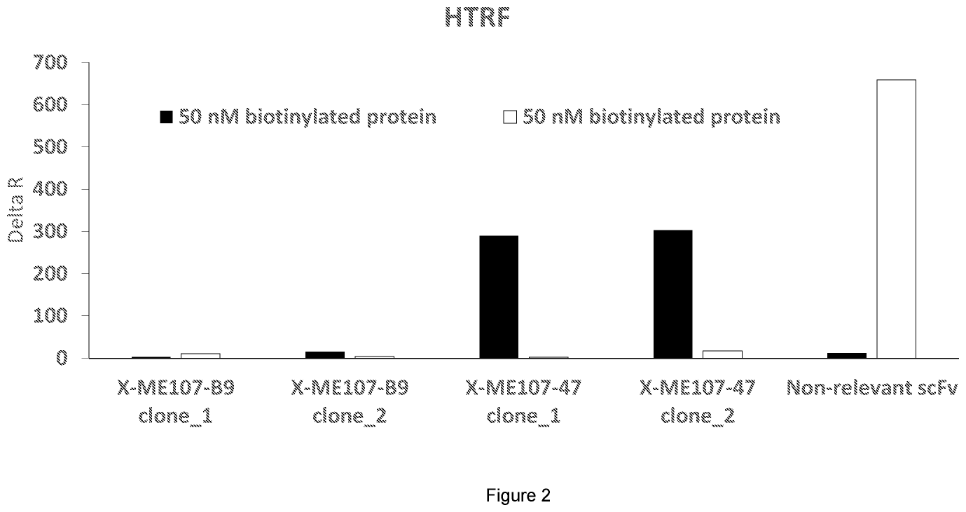

The results of these analyses showed that mAb47 scFv recognized human IL13Rα2 (FIGS. 1 and 2). 1E10B9 scFv did not give rise to a detectable signal to any of the included antigens, neither in ELISA nor HTRF.

### Conclusion

scFv genes encoding the VH and VL of the IL13Rα2 specific mouse IgG antibodies 1E10B9 and mAb47 were successfully synthesized and cloned into pHAT6 and the proteins expressed and analyzed for binding by ELISA and HTRF.

mAb47 scFv showed retained binding to human IL13Rα2 and no binding to the negative control proteins was detected.

1E10B9 scFv, however, did not show binding to human IL13Rα2. This is in contrast to what has been reported for its full length IgG counterpart (Kim et al., A novel single-chain antibody redirects adenovirus to IL13Rα2-expressing brain tumors, Sci Rep 2015, 5: 18133). Loss of antigen binding when alternating between antibody formats is not uncommon, especially when going from IgG to scFv. An altered protein fold affecting the structure of the antigen-binding site can most likely explain this.

### Example 2—Phage Display Selection on Human and Mouse IL13Rα2 Using Phage Libraries

Phage display selections were performed to enable isolation of scFv fragments with specificity for human and mouse IL13Rα2. The scFv were selected from two different human phage libraries (SciLifeLib 1 and 2). A primary screen of a total of 920 clones by ELISA resulted in 673 positive clones being sent for sequencing. 304 of these turned out to be sequence unique.

Material and Methods

Antigens

The human and mouse versions of IL13Rα2 used as antigen for phage display selection are listed in Table 7.

TABLE 7
IL13Rα2 reagents

| | | | | Conc. | Mw |
|---|---|---|---|---|---|
| Name | Short name | Source | Characteristics | (mg/ml) | (kDa) |
| Biotinylated | hIL13Rα2-avi | Example A1 | Produced in Sf9-cells. In | 0.82 | 42.2 |
| human | | | vivo biotinylated on avi-tag | | |
| IL13Rα2 | | | | | |
| Mouse | mIL13Rα2-Fc | RnD Systems | Produced in NS0-cells. | 0.1 | 128 |
| IL13Rα2 | | (#539-IR) | Homodimer through Fc- | | |
| Fc chimera | | | fusion | | |

Phage Display Selection

Biopanning was performed using four selection rounds of enrichment employing two human synthetic scFv phage libraries, SciLifeLib1 and SciLifeLib2 (SciLifeLab, Stockholm, Sweden). SciLifeLib 1 and 2 are naive human synthetic scFv libraries, similar in design and construction to previously reported (Säll, et al., Protein Eng Des Sel (2016) 29: 427-437). Briefly, human germline genes IGHV3-23 and IGKV1-39 were used as library scaffold and Kunkel mutagenesis was used to introduce diversity into four of the six complementarity determining regions (CDR); namely CDR-H1, CDR-H2, CDR-H3 and CDR-L3. For the biotinylated sample (hIL13Rα2-avi), the selection was performed using streptavidin-coated magnetic beads (Dynabeads M-280, ThermoFisher Scientific, #11206D). Analogously, protein G-coupled magnetic beads (Dynabeads, ThermoFisher Scientific, #10004D) were used to capture the Fc-fused mIL13Rα2-Fc. In two of the tracks, in order to preferentially select for cross-species reactive scFv, the antigen was alternated between human and mouse IL13Rα2 in the different rounds. Furthermore, in another two tracks, human IL-13 (Prospec #cyt-446) was included in the selection buffer. The selection pressure was increased by gradually decreasing the antigen amount and by increasing the number and intensity of washes between the different rounds. Elution of antigen-bound phages was performed using a trypsin-aprotonin approach. The entire selection process, except the phage-target protein incubation step, was automated and performed with a Kingfisher Flex robot. The combination of the different parameters resulted in a scheme covering a total of six different selection tracks.

Re-Cloning and Expression of scFv

To allow production of soluble scFv, phagemid DNA from the third and fourth round of each selection track was isolated. In pools, the genes encoding the scFv fragments were restriction enzyme digested and sub-cloned into screening vector pHAT-6, providing a signal for secretion of the scFv along with a triple-FLAG tag and a hexahistidine (His₆) tag at the C-terminus. The constructs were subsequently transformed into TOP10 E. coli. Single colonies were picked, cultivated and IPTG-induced for soluble scFv expression in 96-well format. In total, 920 scFv clones present in bacterial supernatant were prepared for a primary ELISA screen.

ELISA Screen

Mouse IL13Rα2-Fc was directly coated into a 384-ELISA well plate at 1 μg/ml in PBS, 4° C. overnight, whereas human IL13Rα2-avi were indirectly coated through streptavidin, also at 1 μg/ml in PBS, 4° C. overnight. Two negative control proteins, streptavidin and BSA, were also coated.

FLAG-tagged W-ME107 scFv clones present in bacterial supernatant were diluted 1:3 in block buffer (PBS supplemented with 0.5% BSA+0.05% TWEEN 20®) and allowed to bind to the coated proteins. Detection of binding was enabled through an HRP-conjugated anti-FLAG M2 antibody (Sigma-Aldrich #A8592) followed by incubation with TMB ELISA substrate (ThermoFisher Scientific #34029). The colorimetric-signal development was stopped by adding 1 M sulfuric acid and the plate was read at 450 nm. All samples were assayed in duplicates. Also the two reference scFv, mAb47 scFv and G-strep-1 scFv, were included.

DNA Sequencing

673 positive scFv clones showing binding to human and/or mouse IL13Rα2 were sent for Sanger DNA sequencing to GATC Biotech (Ebersberg, Germany).

Results

A total of six selection tracks were carried out in parallel on human and/or mouse IL13Rα2 using SciLifeLib 1 and 2. Following re-cloning of selected scFv clones, 92 clones (colonies) were picked from selection rounds 3 and 4, resulting in a total of 920 picked clones.

ELISA screen resulted in the identification of 673 potential hits having distinct binding characteristics. The majority of hits displayed only binding towards human IL13Rα2 with the remaining hits displaying binding towards both human and mouse IL13Rα2.

DNA sequencing of the 673 hits resulted in the identification of 304 sequence unique W-ME107 clones.

### Conclusion

scFv clones binding to human and/or mouse IL13Rα2 were successfully isolated by phage display selections. Following an initial ELISA screen of 920 clones and DNA sequencing of 673 positive hits, a total of 304 sequence unique scFv clones were identified. A majority of the clones bound only to human IL13Rα2 but a set showed binding to both human and mouse IL13Rα2.

### Example 3—Secondary Screen of 304 Sequence Unique scFv Clones by ELISA and HTRF

To facilitate further ranking of the 304 sequence-unique scFv clones, a secondary ELISA screen and a Homogenous Time Resolved Fluorescence (HTRF) screen were performed on all sequence unique clones.

These analyses confirmed the results of the initial ELISA screen (Example 2) and two major groups of clones, based on binding specificity, were identified. One group bound only to human IL13Rα2, while the other group contained clones that bound to both human and mouse IL13Rα2.

The generated ELISA and HTRF data was used to reduce the number of clones further, resulting in a list of 160 scFv. These 160 clones were chosen based on two main criteria; 1) showing high signal on both mouse and human IL13Rα2 (cross-species reactive) or 2) displaying high signal on human IL13Rα2 and low background signal to non-related targets.

Material and Methods

ELISA

Human IL13Rα2-avi was in-directly coated into 384 ELISA well plates through streptavidin, whereas mouse IL13Rα2-Fc and negative control proteins streptavidin and a non-relevant protein were directly coated to the wells. Details on the IL13Rα2 proteins used in this Example are given in Table 7.

Following coating of plates overnight at 4° C., the plates were washed twice with MilliQ water and blocked for 2 h in blocking buffer (PBS supplemented with 0.5% BSA+0.05% TWEEN 20®). Triple-FLAG-tagged W-ME107 scFv clones present in bacterial supernatant were diluted 1:10 in blocking buffer and allowed to bind. Detection of binding was enabled through an HRP-conjugated anti-FLAG M2 antibody (Sigma-Aldrich #A8592) followed by incubation with 1-step Ultra TMB ELISA substrate (ThermoFisher Scientific #34029). The colorimetric-signal development was stopped by adding 1 M sulfuric acid and plates were analyzed at 450 nm.

All samples were assayed in duplicates. Also the two positive controls, mAb47 scFv and G-strep-1, were included.

HTRF

W-ME107 scFv clones, as well as references mAb47 scFv and G-strep-1 scFv were diluted 1:5 in assay buffer (PBS supplemented with 0.1% BSA) and allowed to bind to hIL13Rα2-avi or to non-relevant antigen diluted to 200 nM in assay buffer. Detection of binding was enabled through donor molecule terbium-conjugated anti-FLAG antibody (Cisbio #611FG2TL) and acceptor molecule streptavidin-conjugated XL665 (Cisbio #610SAXL). Plates were incubated dark for 2 h at room temperature before being analyzed on an Envision spectrometer (Perkin Elmer) at 615 nm (background/noise signal) and 665 nm (binding signal). The 665 nm value was divided with the 615 nm value to retrieve an R value for each sample.

All samples were assayed in duplicates.

Results

ELISA

The scFv clones could be divided into two main groups based on binding specificity. The first group, containing the majority of scFv, bound only to human IL13Rα2, while clones belonging to the second group bound to both human and mouse IL13Rα2. Reference clone mAb47 scFv showed only binding to human IL13Rα2.

HTRF

The 304 scFv clones were also analyzed for binding to human IL13Rα2 in a FRET-based homogenous solution assay. Also in this assay, the far majority of clones showed a clear binding to human IL13Rα2, with no significant binding to the negative control protein included in the assay. Unexpectedly, mAb47 scFv displayed a quite low binding signal.

### Conclusion

ELISA and HTRF screens were successfully performed on the 304 sequence-unique scFv clones isolated by phage display selection. Based on the results, together with the results from the performed Luminex assay on the same samples in Example 4, 160 clones were selected for further screening by SPR. These 160 clones could be divided into two subsets based on their binding characteristics; i) clones binding to only human IL13Rα2 and ii) clones binding to both human and mouse IL13Rα2.

### Example 4—Evaluating Binding Specificity of 304 scFv Clones by Luminex

In this Example, all 304 unique scFv clones, as well as reference clone mAb47 scFv, were further evaluated for specificity in a Luminex-based approach.

This analysis showed that eight of the 304 clones displayed unspecific binding towards several non-relevant proteins. Remaining 296 clones, as well as reference clone mAb47, displayed specific binding towards their intended antigen, human IL13Rα2.

Together with the LUMINEX® data presented in this Example 4, the generated ELISA and HTRF data on the same samples in Example 3 was used to reduce the number of clones further, resulting in a list of 160 scFv. In addition to good performance in LUMINEX®, these 160 clones were chosen based on two main criteria; 1) showing high signal on both mouse and human IL13Rα2 (cross-species reactive) or 2) displaying high signal on human IL13Rα2 and low background signal to non-related targets.

Material and Methods

Biotinylated human IL13Rα2-avi and 31 biotinylated non-relevant proteins were individually conjugated to a specific neutravidin-coupled Luminex bead ID. Following conjugation, all bead IDs were mixed and incubated with scFv clones present in bacterial supernatant, diluted 1:10 in assay buffer (PBS supplemented with 3% BSA, 0.05% TWEEN 20® and 10 μg/ml neutravidin). For each of the 31 non-relevant proteins, at least one positive control scFv was included. Binding of clones to a particular protein-conjugated bead was enabled through an R-PE-conjugated anti-FLAG M2 (Prozyme #PJ315) antibody followed by analyses on a FlexMAP 3D instrument.

All samples were assayed in duplicates and a mean value of obtained binding signal, corresponding to median intensity fluorescence (MFI), was calculated for each clone towards each bead ID.

Results

296 out of the 304 scFv clones screen displayed specific binding towards human IL13Rα2. Eight clones however, besides binding to human IL13Rα2, showed binding to several non-relevant proteins.

Of note, for each of the 31 non-relevant proteins, at least one positive control scFv was assayed. All of these specifically interacted with their cognate antigen as expected. This ensures that all antigens were functionally coupled to their respective beads.

### Conclusion

A 32-plexed LUMINEX® based assay was successfully performed on the 304 sequence-unique scFv clones isolated by phage display selection. Only eight of the scFv showed unspecific binding towards non-relevant proteins included in the assay. The remaining 296 clones, and reference clone mAb47, displayed specific binding towards human IL13Rα2 with no significant binding to any of the 31 non-relevant proteins.

### Example 5—Kinetic Screen of 160 Sequence Unique scFv by SPR

160 W-ME107 scFv clones from Example 4, as well as reference clone mAb47 scFv, were selected for further characterization by surface plasmon resonance (SPR) in a kinetic screen-based approach to enable efficient ranking of the different clones.

Material and Methods

The kinetic screen was performed on a BIACORE® T200 instrument (GE Healthcare). An anti-FLAG M2 antibody (Sigma-Aldrich #F1804), functioning as a capture ligand, was immobilized onto all four surfaces of a CM5-S amine sensor chip according to manufacturer's recommendations.

160 FLAG-tagged W-ME107 clones present in bacterial supernatant were injected and captured onto the chip surfaces, followed by injection of either hIL13Rα2-avi, hIL13Rα2-Fc or mIL13Rα2-Fc at 50 nM. Details on the IL13Rα2 proteins used in this study are given in Table 8. The surfaces were regenerated with 10 mM glycin-HCl pH 2.2. All experiments were performed at 25° C. in running buffer (HBS supplemented with 0.05% TWEEN 20®, pH 7.5).

By subtracting the response curve of a reference surface, being an anti-FLAG M2 antibody immobilized surface, response curve sensorgrams for all scFv clones were obtained. Data was analyzed using the BIACORE® T200 Evaluation 3.1 software.

TABLE 8
IL13Rα2 reagents

| | | | | Conc. | Mw |
|---|---|---|---|---|---|
| Name | Short name | Source | Characteristics | (mg/ml) | (kDa) |
| Biotinylated | hIL13Rα2-avi | Example A1 | Produced in Sf9-cells. In | 0.82 | 42.2 |
| human | | | vivo biotinylated on avi- | | |
| IL13Rα2 | | | tag. Antigen aa E29- | | |
| | | | W331. | | |
| Human | hIL13Rα2-Fc | RnD Systems | Produced in CHO-cells. | 0.1 | 128 |
| IL13Rα2 Fc | | (#7147-IR) | Homodimer through Fc- | | |
| chimera | | | fusion. Antigen aa C22- | | |
| | | | L342. | | |
| Mouse | mIL13Rα2-Fc | RnD Systems | Produced in NS0-cells. | 0.1 | 128 |
| IL13Rα2 | | (#539-IR) | Homodimer through Fc- | | |
| Fc chimera | | | fusion. Antigen aa L22- | | |
| | | | K334. | | |

An anti-FLAG M2 antibody was immobilized onto all four surfaces of a CM5 S chip and similar RU-levels of captured scFv clones were obtained. Following IL13Rα2 injection at 50 nM, each surface was successfully regenerated using a low pH acid solution.

Analysis of data was performed by visual inspection of the sensorgrams (not shown). It was clear from the sensograms that the clones could be divided into three groups based on binding characteristics; (i) scFv clones binding to both human IL13Rα2-avi and human IL13Rα2-Fc, as well as mouse IL13Rα2-Fc; (ii) scFv binding to both human IL13Rα2 variants but showing no measurable binding to mouse IL13Rα2-Fc; (iii) scFv binding to only human IL13Rα2-avi but not to human IL13Rα2-Fc, a few of which also displayed binding towards mouse IL13Rα2-Fc.

The binding patterns for 44 of the 160 scFv are listed in Table 9. This set was considered the most promising based on binding to the human receptor. More specifically, a high binding response and favorable slow off-rate were considered. In addition, clones showing cross-species reactivity were included.

TABLE 9
Binding of 44 scFv to three different IL13Rα2 protein constructs

| | | | |
|---|---|---|---|
| Clone name | Binding to hILRα2-avi | Binding to hILRα2-Fc | Binding to mILRα2-Fc |

Cluster (i) scFv clones binding to human IL13Rα2-avi tag, human IL13Rα2-Fc, mouse IL13Rα2-Fc

| | | | |
|---|---|---|---|
| W-ME107-7 | +++ | + | ++ |
| W-ME107-16 | ++ | +++ | +++ |
| W-ME107-26 | ++ | + | +++ |
| W-ME107-52 | ++ | + | +++ |
| W-ME107-55 | +++ | + | ++ |
| W-ME107-67 | ++ | + | +++ |
| W-ME107-75 | ++ | +++ | + |
| W-ME107-88 | + | ++ | +++ |
| W-ME107-129 | + | + | ++ |
| W-ME107-150 | +++ | + | ++ |
| W-ME107-155 | ++ | + | ++ |
| W-ME107-157 | ++ | + | ++ |

Cluster (ii) scFv clones binding to human IL13Rα2-avi and human IL13Rα2-Fc but no detectable
binding to mouse IL13Rα2-Fc

| | | | |
|---|---|---|---|
| mAb47 scFv | +++ | + | − |
| W-ME107-18 | + | + | − |
| W-ME107-32 | + | ++ | − |
| W-ME107-58 | + | ++ | − |
| W-ME107-89 | + | ++ | − |
| W-ME107-106 | ++ | +++ | − |
| W-ME107-107 | + | + | − |
| W-ME107-112 | +++ | + | − |
| W-ME107-117 | ++ | +++ | − |
| W-ME107-123 | ++ | + | − |
| W-ME107-124 | + | + | − |
| W-ME107-125 | ++ | + | − |
| W-ME107-127 | + | + | − |
| W-ME107-128 | ++ | ++ | − |
| W-ME107-130 | ++ | + | − |
| W-ME107-131 | ++ | ++ | − |
| W-ME107-132 | ++ | ++ | − |
| W-ME107-149 | ++ | + | − |
| W-ME107-151 | + | + | − |
| W-ME107-156 | ++ | +++ | − |
| W-ME107-159 | + | ++ | − |

Cluster (iii) scFv clones inding to human IL13Rα2-avi but no detectable binding to human
IL13Rα2-Fc. A few scFv clones al so display binding towards mouse IL13Rα2-Fc.

| | | | |
|---|---|---|---|
| W-ME107-10 | +++ | − | + |
| W-ME107-27 | +++ | − | +++ |
| W-ME107-31 | + | − | − |
| W-ME107-35 | ++ | − | − |
| W-ME107-97 | ++ | − | − |
| W-ME107-101 | ++ | − | − |
| W-ME107-110 | ++ | − | − |
| W-ME107-137 | ++ | − | + |
| W-ME107-141 | ++ | − | − |
| W-ME107-143 | ++ | − | ++ |
| W-ME107-148 | ++ | − | − |
| W-ME107-152 | + | − | − |

Value reported as “−” indicates that no binding could be detected
Value reported as “+” indicates relatively low binding response
Value reported as “++” indicates relatively high binding response but fast off rate
Value reported as “+++” indicates good binding response with favorable slow off rate

### Conclusion

The kinetic screen resulted in clones showing different binding characteristics; (i) clones binding to both human IL13Rα2-avi and Fc-fused human IL13Rα2 (IL13Rα2-Fc), as well as to mouse IL13Rα2-Fc, (ii) clones binding to both human IL13Rα2 variants but showing no measurable binding to mouse IL13Rα2-Fc and (iii) clones binding to only human IL13Rα2-avi but showing no measurable binding to human IL13Rα2-Fc, a few of which also displayed binding towards mouse IL13Rα2-Fc. The generated data allowed for efficient ranking of the different clones and further reduction of the list of clones from 160 to 44.

### Example 6—Small-Scale Protein Purification and ELISA Analyses of 44 scFv Clones

44 clones and reference clone mAb47 scFv were selected, based on the results presented in Examples 3 to 5, for small-scale protein production and purification.

Protein purification was carried out in a 96-well format on a Kingfisher Flex instrument using protein A- or nickel-coupled magnetic beads. All 44 W-ME107 scFv clones could be purified with satisfying purities and at sufficient protein concentrations. Reference clone mAb47 scFv, however, expressed poorly and thereby displayed low purity and concentration.

When analyzed by ELISA, most purified clones displayed binding to their expected antigen, human and/or mouse IL13Rα2.

Material and Methods

Production and Purification

Each of the 44+1 scFv clones were produced in 15 ml E. coli cultures. Following protein production at 30° C. for 18 h, cells were lysed by B-PER reagent (ThermoFisher Scientific #78248) and clarified bacterial lysates of W-ME107 scFv clones and reference clone mAb47 scFv were mixed with Protein A-coupled magnetic beads (ThermoFisher Scientific #88846) or MagneHis Ni-particle magnetic beads (Promega #V8548), respectively, to enable purification on a Kingfisher Flex instrument. Buffer of eluted scFv clones were exchanged to PBS using a Zeba 96-well spin desalting plate (ThermoFisher Scientific #89807).

Purified scFv clones were analyzed by gel electrophoresis under reducing conditions to determine purity and integrity, and protein concentrations were determined by standard BCA (Bicinchoninic Acid) assay according to manufacturers recommendations (ThermoFisher Scientific #23227).

ELISA

Human IL13Rα2-avi, human IL13Rα2-Fc and mouse IL13Rα2-Fc were coated at 37° C. for 45 min, 1 μg/ml in PBS, into a 384-ELISA well plate. Details on the IL13Rα2 proteins used in this Example are given in Table 8. Three negative control proteins (streptavidin, BSA and a non-relevant protein) were also coated. FLAG-tagged scFv were diluted to 1 μg/ml in blocking buffer (PBS supplemented with 0.5% BSA+0.05% TWEEN 20®) and were allowed to bind to the coated proteins. Detection of binding was enabled through an HRP-conjugate anti-FLAG M2 antibody (Sigma-Aldrich #A8592) followed by incubation with TMB ELISA substrate (ThermoFisher Scientific #34029). The colorimetric-signal development was stopped by addition of 1 M sulfuric acid and the plate was read at 450 nm.

All samples were assayed in duplicates, including reference clone mAb47 scFv and positive assay control G-Strep-1 scFv (specific for streptavidin).

Results

Production and Purification

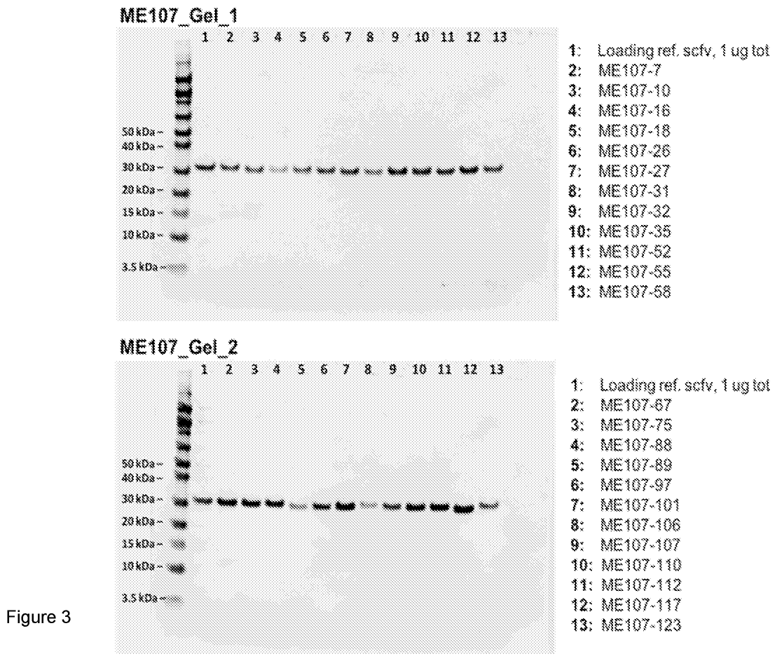

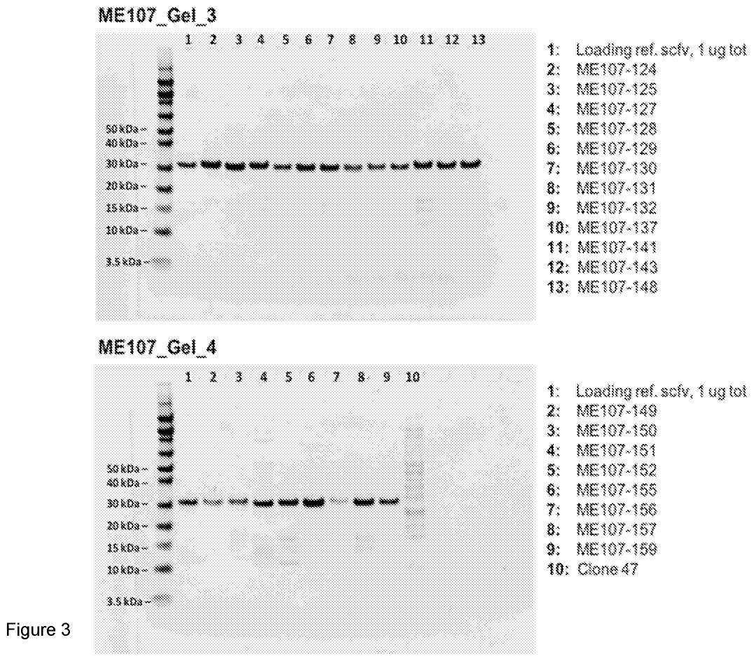

For the 44 W-ME107 scFv, SDS-PAGE showed satisfying sample purity, i.e., one main band correlating to the expected molecular weight of a scFv (approximately 30 kDa) and with no or only a few weak E. coli-derived protein bands (FIG. 3).

Reference clone mAb47, however, displayed low sample purify with multiple E. coli-derived protein bands. Also, the protein band corresponding to its expected molecular weight was quite weak in comparison to all other bands on the gel, indicating a low sample concentration of this clone.

Protein concentrations of purified scFv samples were determined by a standard BCA assay. All 44 W-ME107 scFv clones were purified at sufficient protein concentrations, ranging between 0.1-0.6 mg/ml.

ELISA

The far majority of purified W-ME107 scFv clones displayed binding to their intended antigen, human and/or mouse IL13Rα2 with no or very low binding to coated negative control proteins. There were, however, some clones displaying low or no antigen binding.

### Conclusion

44 W-ME107 scFv clones and reference clone mAb47 were purified in small scale on a Kingfisher flex instrument. All scFv clones, except reference clone mAb47, showed satisfying sample purities and sufficient protein concentrations.

When assayed in an ELISA-based approach, the majority of clones displayed binding to their intended antigen, human and/or mouse IL13Rα2. A few clones however did not perform as expected, having low or no antigen binding.

### Example 7—Investigating Binding Towards Human IL13Rα1 of 44 scFv Clones by ELISA

44 clones and reference clone mAb47 scFv selected for small-scale protein purification in Example 6 were tested for binding to human IL13Rα1 using an ELISA-based approach.

Result showed that all W-ME107 scFv clones displayed binding to their intended antigen, human and/or mouse IL13Rα2, with no significant binding toward human IL13Rα1. The same result was obtained for reference clone mAb47 scFv.

Material and Methods

Coating proteins hIL13Rα2-avi (Example A1), hIL13Rα2-Fc (RnD Systems #7147-IR), mIL13Rα2-Fc (RnD Systems #539-IR) and hIL13Rα1-Fc (RnD Systems #146-IR) were diluted to 1 μg/ml in PBS and directly coated into a 384-well ELISA plate. Two negative control proteins, streptavidin and a non-relevant protein, were also coated. Following incubation of plates with coating-proteins overnight at 4° C., plates were washed twice with MilliQ water and blocked for 2 h in blocking buffer (PBS supplemented with 0.5% BSA+0.05% TWEEN 20®). Triple-FLAG-tagged W-ME107 scFv clones present in bacterial supernatant were diluted 1:10 in blocking buffer and allowed to bind. Detection of binding was enabled through an HRP-conjugated anti-FLAG M2 antibody (Sigma-Aldrich #A8592) followed by incubation with 1-step Ultra TMB ELISA substrate (ThermoFisher Scientific #34029). The colorimetric-signal development was stopped by addition of 1 M sulfuric acid and plates were analyzed at 450 mm.

All samples were assayed in duplicates, including reference clone mAb47 scFv and positive ELISA control G-Strep-1 scFv (specific for streptavidin).

Results

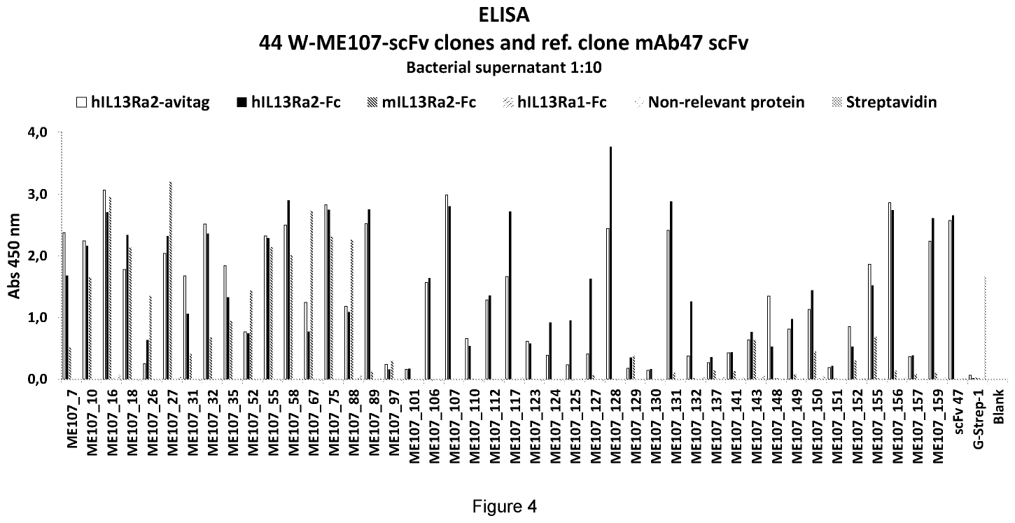

All W-ME107 scFv clones assayed displayed binding towards their intended antigens, human and/or mouse IL13Rα2, as reported previously (Example 3 and Example 5) (FIG. 4). No significant binding could be detected towards human IL13Rα1, or to negative control proteins, for any of the clones.

Reference clone mAb47 scFv only displayed binding towards human IL13Rα2 and not to mouse IL13Rα2 or human IL13Rα1, and positive assay control G-strep-1 scFv bound to streptavidin coated wells.

### Conclusion

No significant binding towards human IL13Rα1 could be detected for any of the 44 W-ME107 scFv clones, or for reference clone mAb47 scFv, when assayed from bacterial supernatant (FIG. 4).

In contrast to the kinetic screen results (Example 5), all clones here showed binding to both human IL13Rα2 proteins; the avi-tagged and the Fc-fused. In contrast, in Example 5, twelve of the 44 clones showed no measureable binding to the Fc-fused variant. This discrepancy can most likely be explained by a difference in sensitivity of the two methods but also on experimental set-up. In this Example, the receptors are attached to a surface, while in Examples 5 and 9 the receptors are in solution. The differences in tags or fusion proteins of the receptor could have a large influence on how the antigens behave and different epitopes may be differently displayed in the two experimental set-ups.

The binding to mouse IL13Rα2 correlated well to the previous SPR data (Example 5), i.e., a scFv showing no measurable binding to the target in the affinity screen was also considered a “non-binder” in this experimental set-up.

A few of the scFv showed low signals to all analyzed proteins (exemplified by W-ME107-97, W-ME107-101, W-ME107-129, W-ME107-130, W-ME107-137, W-ME107-141, W-ME107-151, W-ME107-157) (FIG. 4).

### Example 8—Cell Binding

The binding of selected scFv to cells expressing IL13Rα2 was assessed to confirm binding to the receptor also on the surface of target cells.

Materials and Methods

scFv binding to IL13Rα2 expressing cells was assessed by incubating 100 ng purified scFv (Example 6) with 1×10⁵ cells of the human glioblastoma cell line U-87MG (original Uppsala University clone with verified IL13Rα2 expression, PMID: 27582061) or human non-small cell lung cancer cells (A549) for 20 min in room temperature (RT) (about 20-25° C.). The human glioblastoma cell line U-87MG endogenously expresses high levels of human IL13Rα2 (hIL13Rα2), whereas the human non-small cell lung cancer cells do no not express hIL13Rα2.

After washing (1×PBS, 0.1% BSA, 3 mM EDTA), FLAG-tag was stained using a PE conjugated anti-FLAG antibody for 20 min in RT. Readout was done using CytoFLEX Flow cytometer (Beckman coulter, CA).

Results

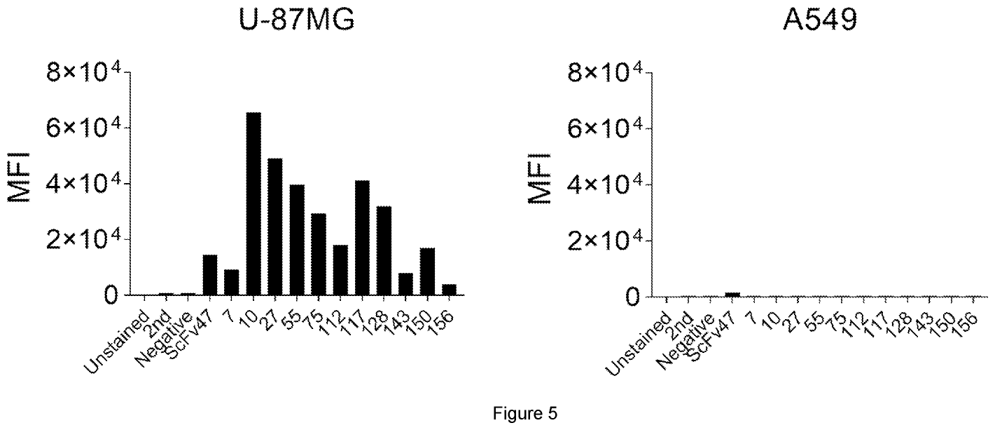

Selected scFv were incubated with target cells expressing IL13Rα2, to confirm binding to the receptor also on the surface of target cells. All scFv could specifically bind to the human glioblastoma cell line U-87MG endogenously expressing high levels of hIL13Rα2 but did not bind to negative control human non-small cell lung cancer cells (A549) (FIG. 5).

### Conclusion

Flow cytometry successfully assessed the cell binding ability of the selected W-ME107 clones, and the selected W-ME107 clones showed different binding avidities to the target cell line. No off-target, i.e., binding to non-glioma cell, was observed in this binding assay.

### Example 9—Single Cycle Kinetics by SPR on 11 W-ME107 scFv Clones

Surface plasmon resonance (SPR) analyses using a single cycle kinetic approach were performed on the 11 most promising W-ME107 clones, selected based on the results of Examples 5, 7 and 8, and reference clone mAb47 scFv to determine their kinetic parameters for human IL13Rα2-avi, human IL13Rα2-Fc and mouse IL13Rα2-Fc, respectively.

Analyses of obtained data showed the highest affinity-binding clones displaying K_{D}-values in the low nanomolar range towards both human and mouse IL13Rα2.

Material and Methods

An anti-FLAG M2 antibody (Sigma-Aldrich #F1804), functioning as a capture ligand was immobilized onto all four surfaces of a CM5-S amine sensor chip on a BIACORE® T200 instrument (GE Healthcare) according to manufacturer's recommendations.

Protein A purified FLAG-tagged W-ME107 clones were each injected and captured onto the chip surface, aiming for equal response units (RU) between clones. Reference clone mAb47 was, however, captured non-purified, from the bacterial supernatant, since protein purification resulted in too low yield (Example 6).

A 3-fold dilution series of human IL13Rα2-avi, human IL13Rα2-Fc and mouse IL13Rα2-Fc, respectively, consisting of five concentrations ranging between 1.2-100 nM, were prepared in running buffer (HBS supplemented with 0.05% TWEEN 20®, pH 7.5) and sequentially injected over the chip surfaces. Following a dissociation phase, the chip surfaces were regenerated with 10 mM glycine-HCl, pH 2.1.

By subtracting the response curve of a reference surface, being an anti-FLAG M2 antibody immobilized surface, response curve sensorgrams for all scFv clones were obtained. Data was analyzed using software BIAeval version 3.1 (GE Healthcare) and kinetic parameters were calculated assuming a 1:1 Langmuir binding model.

Results

Data showed that all W-ME107 clones displayed an affinity in the low nanomolar to nanomolar range for human hIL13Rα2-avi, with the highest affinity binding clone, W-ME107-27, having a K_{D}-value of 3.1 nM (Table 10).

All but four clones (W-ME107-112, W-ME107-117, W-ME107-128 and W-ME107-156) bound also to the mouse version of the receptor. This correlated well to the data presented in Example 5.

Unexpectedly, while all of the analyzed clones clearly showed binding to hIL13Rα2-avi, only about half showed binding to the Fc-fused human construct (hIL13Rα2-Fc). This was exemplified by W-ME107-10, W-ME107-27, W-ME107-55, W-ME107-112, W-ME107-143 and W-ME107-150 that showed a high response against hIL13Rα2-avi (>50 RU) but hardly any response to the Fc-fused human construct. This was also noted during kinetic screen analyses (Example 5).

Binding affinity, K_{D} (M), of reference clone mAb47 scFv (present in bacterial supernatant) to human IL13Rα2-avi was determined to 1.1 nM.

TABLE 10
kinetic parameters of W-ME107 clones and
reference clone mAb47

| | Human ILRα2-avi (Example A1) | | |
|---|---|---|---|
| Clone name | kₐ (1/Ms) | k_{d} (1/s) | K_{D} (nM) |
| W-ME107-7 | 1.1 × 10⁵ | 1.2 × 10⁻³ | 11 |
| W-ME107-10 | 5.1 × 10⁴ | 1.7 × 10⁻⁴ | 3.4 |
| W-ME107-27 | 6.9 × 10⁴ | 2.2 × 10⁻⁴ | 3.1 |
| W-ME107-55 | 6.9 × 10⁴ | 4.6 × 10⁻⁴ | 6.6 |
| W-ME107-75 | 2.2 × 10⁵ | 7.9 × 10⁻³ | 36 |
| W-ME107-112 | 5.2 × 10⁴ | 1.4 × 10⁻³ | 26 |
| W-ME107-117 | 1.5 × 10⁵ | 3.3 × 10⁻³ | 22 |
| W-ME107-128 | 5.4 × 10⁴ | 6.0 × 10⁻³ | 110 |
| W-ME107-143 | 5.2 × 10⁴ | 3.1 × 10⁻³ | 60 |
| W-ME107-150 | 7.8 × 10⁴ | 2.3 × 10⁻³ | 29 |
| W-ME107-156 | 9.9 × 10⁴ | 5.7 × 10⁻³ | 57 |
| mAb47 scFv | 2.2 × 10⁵ | 2.3 × 10⁻⁴ | 1.1 |
| | Human ILRα2-Fc (RnD System) | | |
| Clone name | kₐ (1/Ms) | k_{d} (1/s) | K_{D} (nM) |
| W-ME107-7 | 9.8 × 10³ | 3.4 × 10⁻⁴ | 34 |
| W-ME107-10 | ** | ** | ** |
| W-ME107-27 | ** | ** | ** |
| W-ME107-55 | ** | ** | ** |
| W-ME107-75 | 4.8 × 10⁴ | 2.6 × 10⁻⁴ | 5.4 |
| W-ME107-112 | ** | ** | ** |
| W-ME107-117 | 4.5 × 10⁴ | 3.5 × 10⁻⁴ | 7.8 |
| W-ME107-128 | 3.5 × 10⁴ | 7.6 × 10⁻⁴ | 22 |
| W-ME107-143 | ** | ** | ** |
| W-ME107-150 | ** | ** | ** |
| W-ME107-156 | 4.5 × 10⁴ | 3.5 × 10⁻⁴ | 7.8 |
| | Mouse ILRα2-Fc (RnD System) | | |
| Clone name | kₐ (1/Ms) | k_{d} (1/s) | K_{D} (nM) |
| W-ME107-7 | 8.0 × 10⁵ | 3.9 × 10⁻² | 49 |
| W-ME107-10 | 4.0 × 10⁴ | 1.8 × 10⁻³ | 45 |
| W-ME107-27 | 8.0 × 10⁴ | 1.9 × 10⁻⁴ | 2.4 |
| W-ME107-55 | 1.6 × 10⁵ | 3.3 × 10⁻³ | 20 |
| W-ME107-75 | 4.5 × 10⁴ | 3.6 × 10⁻³ | 80 |
| W-ME107-112 | — | — | — |
| W-ME107-117 | — | — | — |
| W-ME107-128 | — | — | — |
| W-ME107-143 | 4.9 × 10⁴ | 1.8 × 10⁻³ | 36 |
| W-ME107-150 | 4.2 × 10⁵ | 3.5 × 10⁻² | 82 |
| W-ME107-156 | — | — | — |

All scFv samples were present as purified samples except clone mAb47 scFv, which was present in bacterial supernatant.
Values reported as “—” indicates that binding parameters could not be determined.
Values reported as “**” indicates inconclusive data due to low binding response (RU).

### Conclusion

A single cycle kinetic approach on 11 W-ME107 scFv clones and reference clone mAb47 was successfully carried out on a BIACORE® T200 instrument. Obtained kinetic parameters of each clone for human IL13Rα2-avi, human IL13Rα2-Fc and mouse IL13Rα2-Fc, respectively, correlated well with estimated affinities obtained during kinetic screen analyses (Example 5). The highest affinity binding clone, W-ME107-27, had a K_{D}-value of 3.1 nM. W-ME107-27 was also the clone displaying the highest affinity for mouse IL13Rα2-Fc (K_{D}=2.4 nM).

Unexpectedly, in this Example, some clones showed good binding to hL13Rα2-avi but not to hIL13R2a2-Fc (W-ME107-10, W-ME107-27, W-ME107-55, W-ME107-112, W-ME107-143 and W-ME107-150). This is despite the fact that the amino acids included in these protein constructs are almost identical (Table 8). However, different epitopes may be displayed differently in the two constructs. For example, dimerization caused by the Fc-fusion may cause steric hindrance of some epitopes.

### Example 10—Tm Determination of 11 W-ME107 scFv Clones by nanoDSF

Eleven purified W-ME107 scFv clones were subjected to melting temperature, Tm (° C.), measurements by nanoDSF on a Prometheus NT.48 instrument in order to evaluate stability.

Material and Methods

The nanoDSF technology measures the intrinsic fluorescence of a protein while it is being subjected to thermal denaturation, thereby characterizing the unfolding of the protein under native conditions.

The protein A purified W-ME107 scFv clones (Example 6) were diluted to 0.1 mg/ml in PBS and loaded into “high sensitivity” capillary (NanoTemper #PR-0006) through capillary force, which were subsequently mounted into a Prometheus NT.48 instrument. Melting temperature ramp was set between 20° C. and 95° C., heating 1° C./min.

Tryptophan emission was measured at 330 nm and 350 nm and the calculated ratio plotted against temperature to obtain melting curves for each clone, from which Tm-values can be deduced using software PR. ThermoControl (NanoTemper Inc).

Results

The melting temperatures were determined by nanoDSF. Table 11 presents Tm-values (inflection points) for each of the 11 scFv clones.

TABLE 11
Melting temperature (Tm) values

| | | Inflection | Inflection |
|---|---|---|---|
| | Onset #1 | Point #1 | Point #2 |
| | for Ratio | for Ratio | for Ratio |
| Clone number | (° C.) | (° C.) | (° C.) |
| W-ME107-7 | 42 | 57 | 70 |
| W-ME107-10 | 56 | 63 | |
| W-ME107-27 | 48 | 55 | |
| W-ME107-55 | 43 | 62 | |
| W-ME107-75 | 61 | 66 | |
| W-ME107-112 | 47 | 66 | |
| W-ME107-117 | 64 | 69 | |
| W-ME107-128 | 66 | 72 | |
| W-ME107-143 | 51 | 57 | |
| W-ME107-150 | 43 | 73 | |
| W-ME107-156 | 60 | 68 | |

### Conclusion

The melting temperature, Tm (° C.) could be determined for all 11 W-ME107 scFv clones. Tm values were in the expected range, approximately 60° C.<Tm<70° C., for all clones except for W-ME107-27 and W-ME107-143, which displayed a somewhat lower Tm value of 55° C. and 57° C., respectively.

Also, all clones displayed only one melting event except W-ME107-7, which displayed two melting events. This may possibly be due to tryptophan-containing contaminants or a heterogenous mix of W-ME107-7 (folded and misfolded populations present).

Determined Tm-values may be used as a protein stability measurement and the obtained data indicates lower stability for clone W-ME107-27 and W-ME107-143 compared to other scFv clones included in this analysis.

### Example 11—IL13-Inhibition of W-ME107 scFv Binding to IL13Rα2 by SPR

Twelve W-ME107 scFv clones and reference clone mAb47 scFv were analyzed for their ability to compete with IL13 for binding to the receptor using an SPR-based approach.

Each W-ME107-clone was captured onto an anti-FLAG M2 antibody immobilized SPR chip surface and allowed to bind to IL13a2 receptor itself, as well as to IL13Rα2 pre-incubated with IL-13.

It was found that seven out of the twelve clones, W-ME107-7, W-ME107-10, W-ME107-27, W-ME107-55, W-ME107-67, W-ME107-112, W-ME107-150, showed IL-13 competition for binding to the receptor. Here, some clones displayed an almost complete loss of binding with IL-13 present, whereas some clones only showed a partial loss of binding to the receptor. Binding of clones to IL13Rα2 pre-incubated with a negative control protein could not block receptor binding. These findings indicate that these seven clones have a binding epitope overlapping with, or in close proximity to, ligand binding site of IL-13 on the receptor.

The five clones that were not affected by the presence of IL-13, namely W-ME107-16, W-ME107-75, W-ME107-117, W-ME107-128, and W-ME107-156, bound an epitope distinct from the IL-13 binding site of IL13Rα2.

Reference clone mAb 47 scFv displayed no binding activity in this experiment so no data could be retrieved. However, in the literature it has been reported that this antibody competes with IL-13 for binding to IL13Rα2 (Balyasnikova et al., Characterization and immunotherapeutic implications for a novel antibody targeting interleukin (IL)-13 receptor α2, J Biol Chem 2012, 287(36): 30215-30227).

Material and Methods

An anti-FLAG M2 antibody (Sigma-Aldrich #F1804), functioning as a capture ligand, was immobilized onto all four surfaces of a CM5-S amine sensor chip through ECD/NHS chemistry on a BIACORE® T200 instrument (GE Healthcare) according to manufacturer's recommendations.

Twelve protein A purified FLAG-tagged W-ME107 clones (Example 6), positive reference clone mAb47 and negative control G-strep-1 scFv (specific for streptavidin), were injected and captured onto the chip surfaces, followed by injection of either 100 nM hIL13Rα2-avi (Example A1), 100 nM hIL13Rα2-avi+200 nM IL13 (Prospec #cyt-446), 100 nM IL13Rα2-avi+200 nM streptavidin (Sigma-Aldrich #5A4762) or 200 nM streptavidin. The surfaces were regenerated with 10 mM glycin-HCl pH 2.1. All experiments were performed at 25° C. in HBS supplemented with 0.05% TWEEN 20®, pH 7.5.

By subtracting the response curve of a reference surface, being an anti-FLAG M2 antibody immobilized surface, response curve sensorgrams for all scFv clones were obtained.

Results

An anti-FLAG M2 antibody was successfully immobilized onto all four surfaces of a CM5 series S chip. Following scFv clone capture and antigen injection, the chip surfaces were successfully regenerated by low pH.

All twelve W-ME107 scFv clones displayed binding towards hIL13Rα2, as well as towards hIL13Rα2 pre-incubated with negative control streptavidin. Also, none of the clones displayed binding towards streptavidin alone.

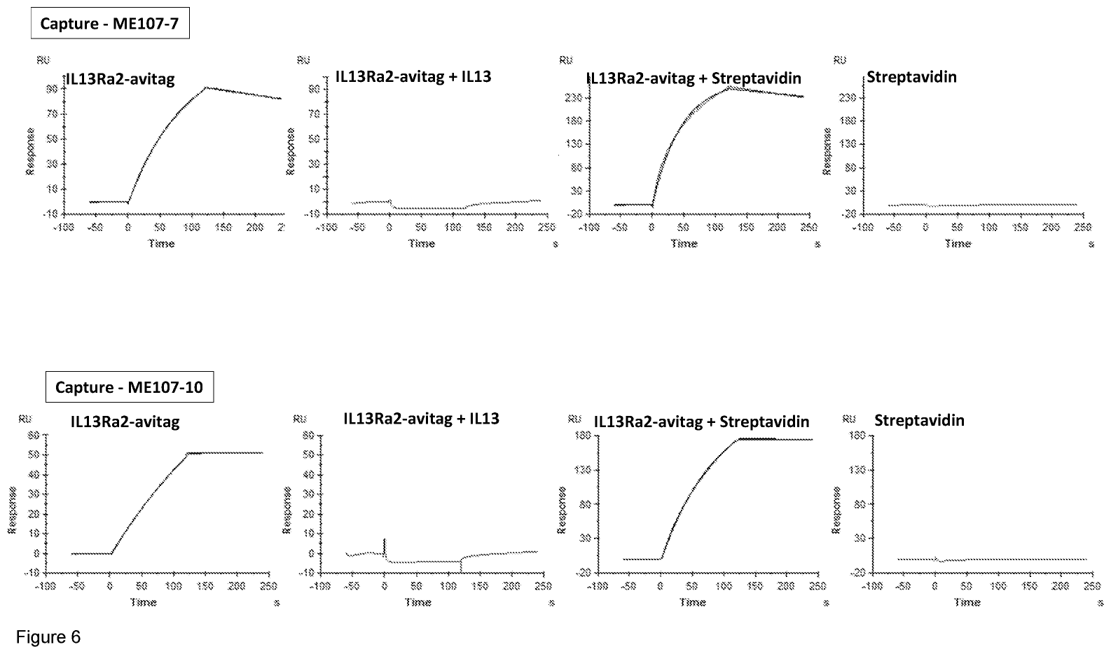

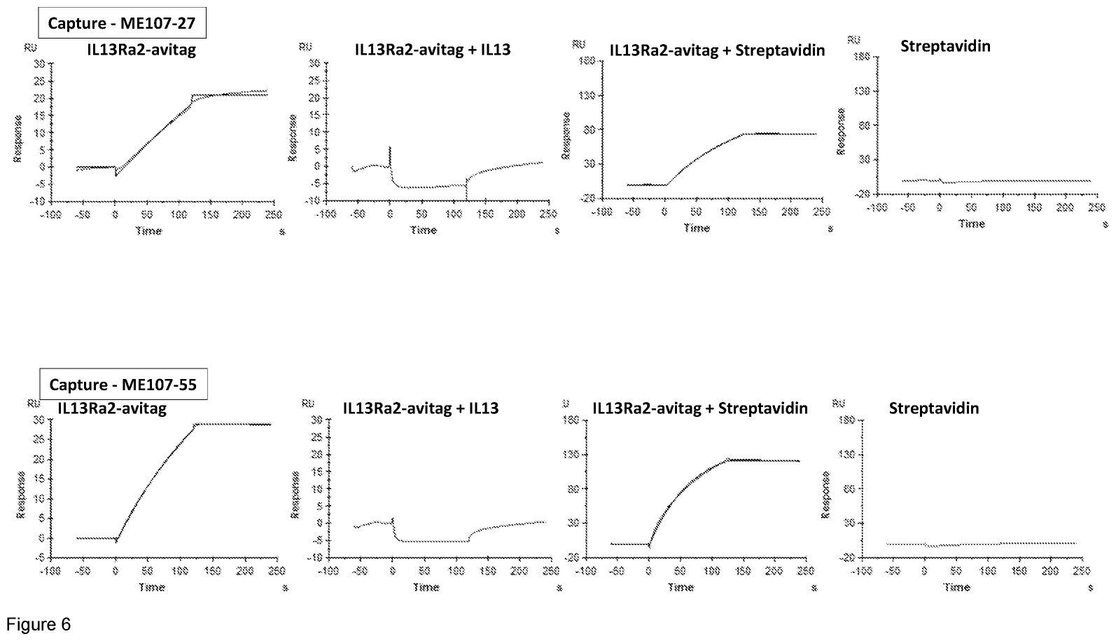

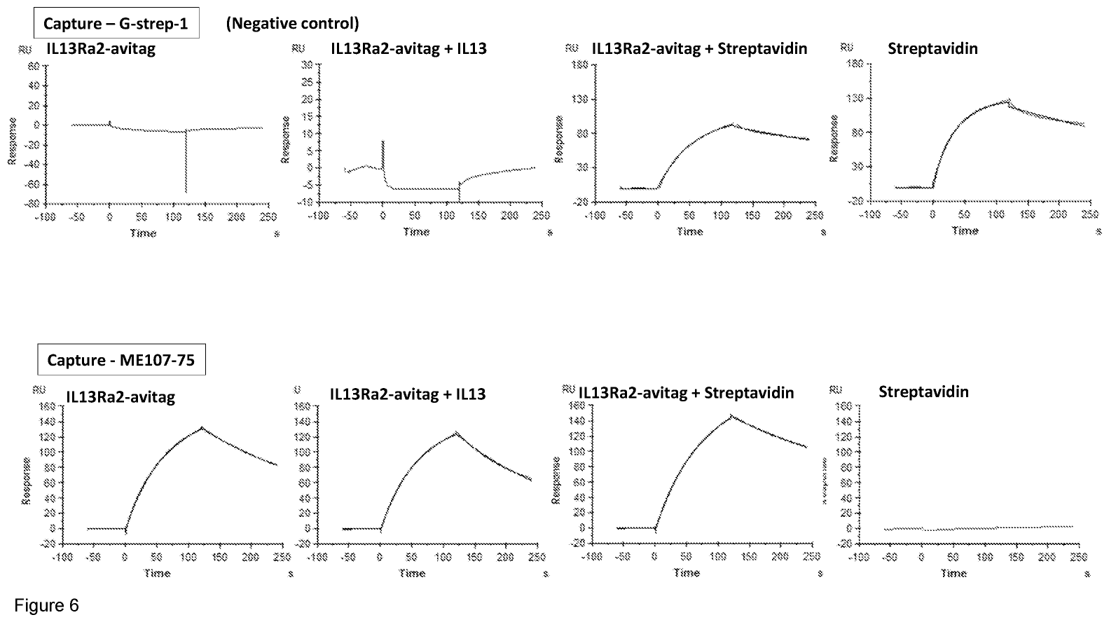

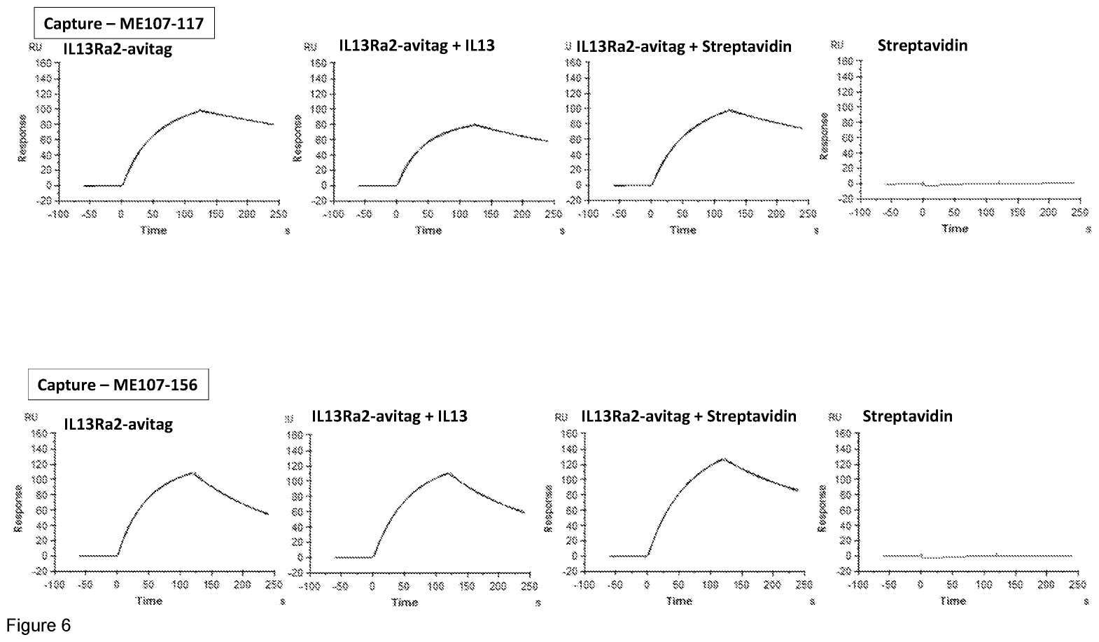

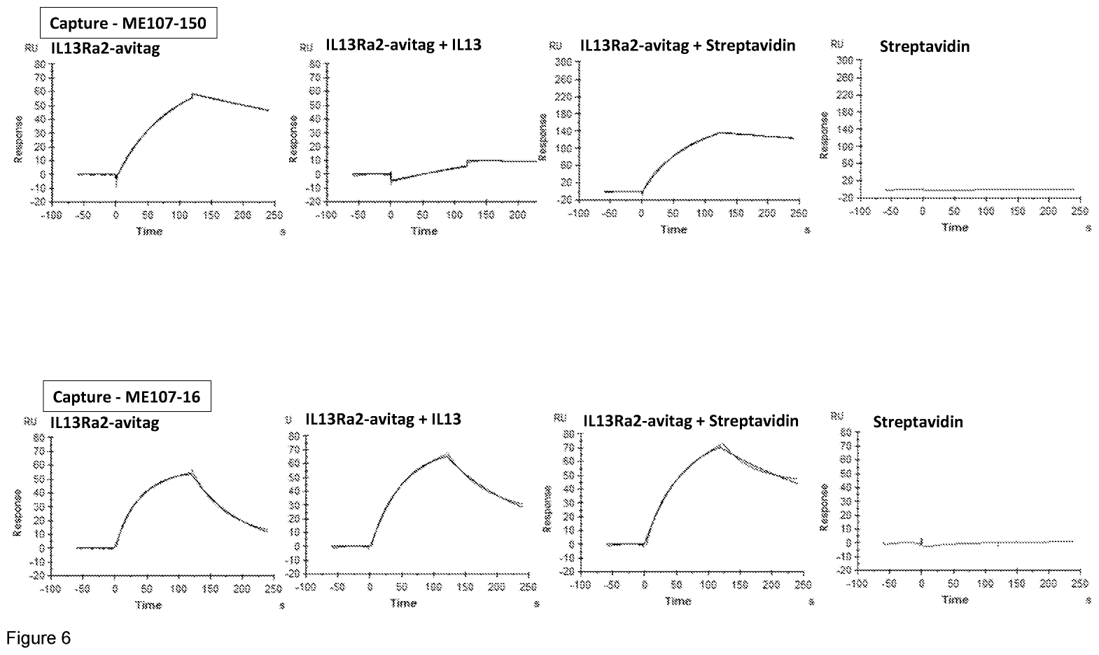

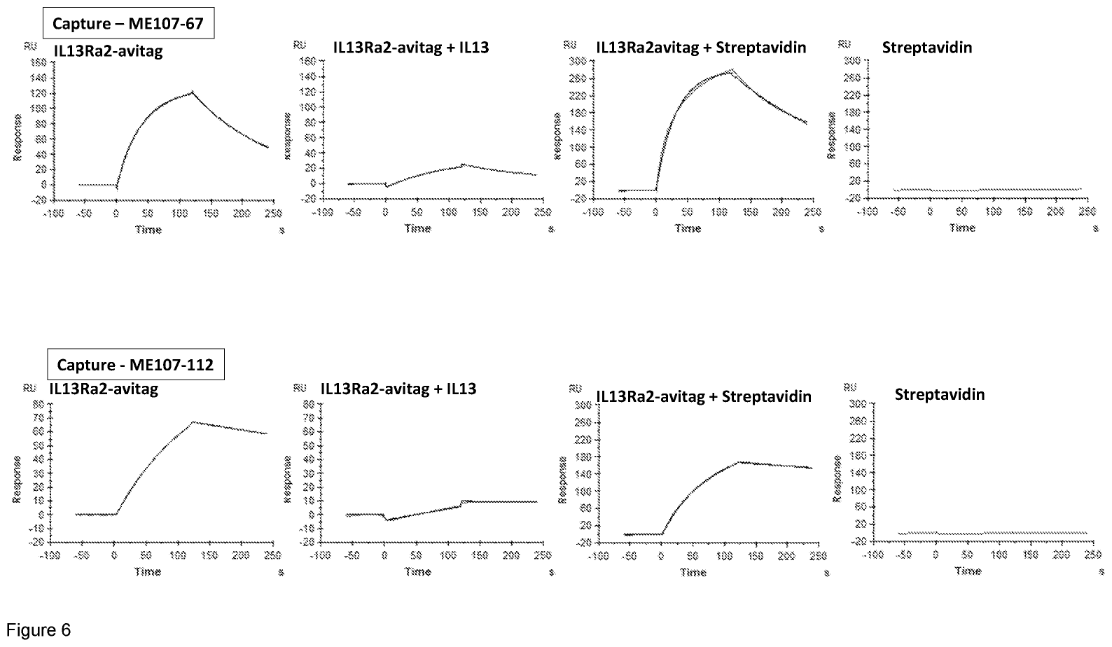

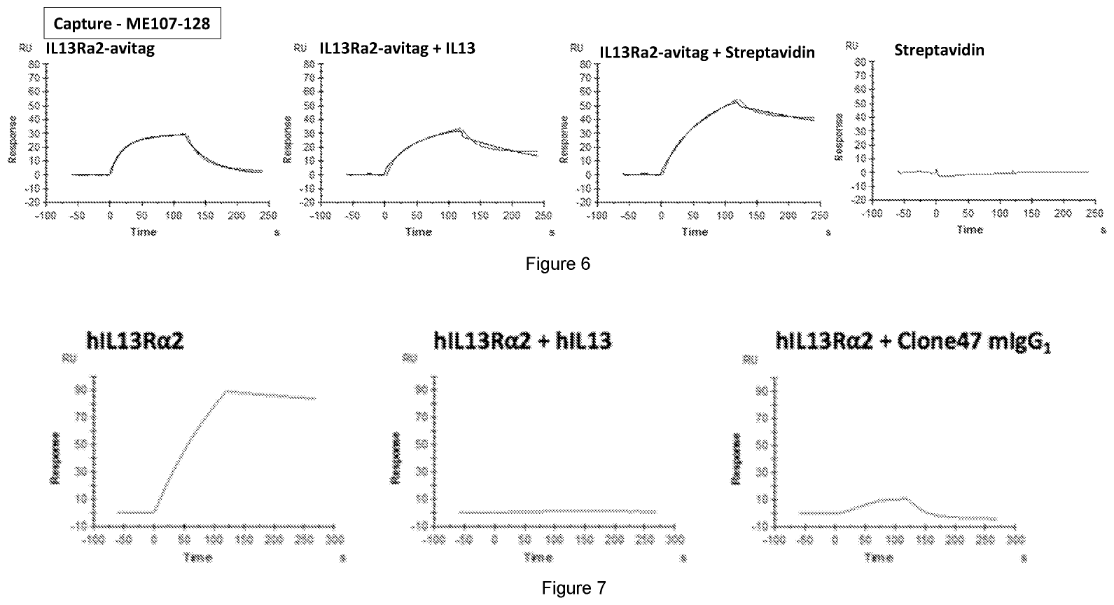

When clones were tested for binding to hIL13Rα2 pre-incubated with IL-13 only five clones (W-ME107-16, W-ME107-75, W-ME107-117, W-ME107-128, and W-ME107-156) showed retained binding towards the receptor-ligand complex (FIG. 6). For the remaining seven clones (W-ME107-7, W-ME107-10, W-ME107-27, W-ME107-55, W-ME107-67, W-ME107-112 and W-ME107-150) binding could be more or less completely blocked by the presence of IL-13, indicated by a decrease in response units (RU) (FIG. 6).

No binding activity of reference clone mAb47 scFv could be seen. As expected, negative control G-strep-1 scFv only displayed binding towards hIL13Rα2 pre-incubated with streptavidin, as well as streptavidin alone.

### Conclusion

The results showed that seven out of the twelve W-ME107 scFv clones could not bind hIL13Rα2 receptor when IL-13 was present. These results indicate that these clones had a binding epitope overlapping with, or in close proximity to, the ligand binding site of IL-13 on the receptor. The remaining five clones were capable of binding to hIL13Rα2 also when IL-13 was bound to the receptor.

All clones showing binding competition with IL-13 also displayed no, or very little, detectable binding to the Fc-fused human IL13Rα2 construct (hIL13Rα2-Fc, RnD systems #7147-IR) in previously performed kinetic measurements (Example 5 and Example 9).

### Example 12—Epitope Binning Experiments Using Clone mAb47 mIgG1

Full-length antibody clone mAb47 mIgG1 was purchased and analyzed in an SPR based epitope binning approach. Also, its kinetic constants towards human IL13Rα2 were determined.

Clone mAb47 mIgG1 displayed an affinity, defined as equilibrium dissociation constant (K_{D} (M)), towards human IL13Rα2 in the sub-nanomolar range, K_{D}=0.9 nM.

Furthermore, epitope binning data showed that clone mAb47 mIgG1 competed or interfered with IL-13, W-ME107-10 scFv and W-ME107-27 scFv for binding to human IL13Rα2. This indicated that they all have overlapping epitopes, or epitopes in close proximity, on the receptor. Data also showed that W-ME107-75 scFv and W-ME107-117 scFv did not interfere with clone mAb47 mIgG1 for binding to human IL13Rα2, hence indicating that they have separate non-overlapping epitopes.

Material and Methods

Single Cycle Kinetics, SCK

Reference clone mAb47 mIgG1 (Creative Biolabs #NEUT-1190QC) was diluted to 50 μg/ml in 10 nM NaAc pH 4.0 and immobilized onto a CM5 series S chip using NHS/EDC chemistry according to manufacturer's recommendations. A 4-fold dilution series of human IL13Rα2-avi, comprised of 5 concentrations, ranging between 50 nM down to 0.2 nM was sequentially injected over the chip surfaces.

Binding sensorgram was subtracted with a blank reference surface sensorgram (no ligand immobilized) and fitted to a 1:1 Langmuir binding model to retrieve the kinetic parameters, kₐ (M⁻¹S⁻¹), k_{d} (S⁻¹) and K_{D} (M).

Epitope Binning

10 nM hIL13Rα2-avi was injected over the immobilized clone mAb47 mIgG1 surface, either alone or pre-incubated with ten times molar excess (100 nM) of human IL13 (Prospec #cyt-446) or W-ME107-10, W-ME107-27, W-ME107-75, W-ME107-117 scFv clones. As control samples, 10 nM hIL13Rα2-avi pre-incubated with ten times molar excess of BI-8 scFv (negative control) or clone mAb47 mIgG1 (positive control) were also assessed. Following an association phase and dissociation phase, the chip surface was regenerated using 10 nM glycin-HCl, pH 2.1. Binding sensorgrams were subtracted with a blank reference surface sensorgram (no antibody immobilized) and binding levels, response units (RU), of each sample could be retrieved.

Results

Clone mAb47 mIgG1 was successfully immobilized onto a CM5 series S chip using NHS/EDC chemistry with retained activity following regeneration with 10 nM glycin-HCl at pH 2.1.

The affinity of clone mAb47 mIgG1 towards hIL13Rα2 was determined to be in the sub-nanomolar range, with a K_{D}-value of 0.9 nM. The observed association rate constant (kₐ) and the dissociation rate constant (k_{d}) were determined to be 5.9×10⁵ M⁻¹s⁻¹ and 5.4×10⁻⁴ s⁻¹, respectively.

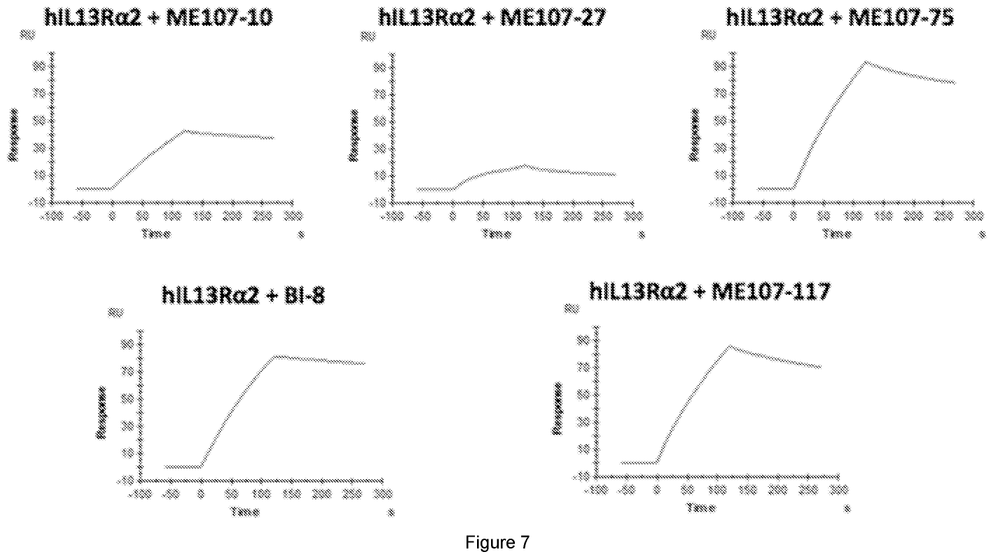

Epitope binning showed that when hIL13Rα2 was pre-incubated with ten times molar excess of human IL-13, W-ME107-10 scFv, W-ME107-27 scFv or positive control clone mAb47 mIgG1, respectively, followed by injection over the clone mAb47 mIgG1 immobilized surface, the observed binding response (RU, y-axis) decreased when compared to injection of hIL13Rα2 alone (FIG. 7). This was not observed for W-ME107-75 scFv, W-ME107-117 scFv and negative control BI-8 scFv, where the binding response was more or less equal to injection of hIL13Rα2 alone (FIG. 7).

### Conclusion

The affinity of reference clone mAb47 mIgG1 towards human IL13Rα2 was determined to be in the sub-nanomolar range (K_{D}-value of 0.9 nM).

Epitope binning results showed that W-ME107-10 and W-ME107-27, as well as human IL-13, competed with reference clone mAb47 mIgG1 for binding to hIL13Rα2 receptor.

Results from the epitope binning analyses correlated well with previously performed SPR analyses on W-ME107 scFv clones (Example 11). This Example showed that W-ME107-10 scFv and W-ME107-27 scFv competed with human IL-13 for binding to hIL13Rα2, whereas W-ME107-75 and W-ME107-117 did not.

Taken together, the results in Examples 11 and 12 showed that W-ME107-10 and W-ME107-27, as well as reference clone mAb47, bound to, or in close proximity to, the human IL-13 binding site on human IL13a2 receptor, whereas W-ME107-75 and W-ME107-117 did not. An overlapping epitope of IL-13 and clone mAb47 is also suggested by Balyasnikova et al., Characterization and immunotherapeutic implications for a novel antibody targeting interleukin (IL)-13 receptor α2, J Biol Chem 2012, 287(36): 30215-30227, where they showed significant inhibition of the interaction between human soluble IL-13 and hIL13Rα2 by this antibody using a plate-based competition assay.

### Example 13—Epitope Mapping Experiments by HDX-MS

When proteins are dissolved in a buffer containing heavy water (D₂O), their hydrogens attached to heteroatoms (e.g., —OH, —NH, —SH) are replaced by deuterium. In hydrogen deuterium exchange mass spectrometry (HDX-MS), the speed of deuterium incorporation is correlated with the degree of intramolecular hydrogen bonding of the peptide backbone amide group (—NHCO—). Backbone amide-NH hydrogens that are already engaged in hydrogen bonding, e.g., —NHCO-hydrogens located at α-helixes or β-sheets, exchange slower than those NH hydrogens available to share their hydrogen with an acceptor. Thus, backbone amide H located in a disordered region will exchange faster than those hydrogens participating in β-sheets or α-helices.

Now, since the binding of a ligand to a protein target produces local changes in hydrogen bonding, e.g., structure stabilization or destabilization, this technique can be used to identify the binding interfaces of protein complexes. For most hydrogens, the H/D exchange is short-lived, and as soon as the polypeptide chain is in contact with a water-containing solution, the deuterons incorporated as -OD, -SD are replaced again by hydrogen. This phenomenon is called “back exchange”, and it is very fast for deuterons bound to heteroatoms located at the side chain of the amino acid residues. Fortunately, under acidic conditions (pH ˜2.3) and near zero ° C. temperatures, the back exchange at the peptide backbone amide group (—NDCO—) slows to time windows compatible with analysis by liquid chromatography and mass spectrometry (LC-MS). In this manner, the degree of incorporation can be monitored by MS since each D atom is one mass unit heavier than an H atom. Additionally, when deuterium labeling is further combined with enzymatic proteolysis, the deuteration profile of different areas within the protein can be monitored.

Materials and Methods

A control sample (IL13Rα2 alone) was prepared by mixing 3 μL of the human IL13Rα2-avi (see Example A1) (1.6 mg/mL) with 24 μL of deuterated PBS. The antigen/scFv complexes were prepared as follows: 40 μL of hIL13Rα2-avi (1.6 mg/mL) was mixed with 34.5 μL of W-ME107-117 scFv (1.46 mg/mL), 74 μL of W-ME107-10 scFv (0.68 mg/mL), 57.2 μL W-ME107-27 scFv (0.88 mg/mL) and 122.8 μL W-ME107-75 scFv (0.41 mg/mL) for a 1:1 antigen/scFv molar ratio, using a 30 kDa average molecular weight for each scFv. The complexes were concentrated to the initial 40 μL using a 10 kDa protein concentrator Amicon Ultra 0.5 mL. The labelling reactions were made by mixing 3 μL of the antigen/scFv complex with 24 μL of deuterated PBS for 4 min, 10 min and 60 min (in triplicate except the 4 min incubations of W-ME107-27 and W-ME107-75, which was done in duplicates). After incubation, the reaction was quenched by decreasing the pH to ˜2.3 and temperature to ˜4° C. by adding 25 μL of a solution containing 6 M Urea, 100 mM TCEP and 0.5% TFA.

Samples were analyzed in an automated HDX-MS system (CTC PAL/Biomotif HDX), in which samples were automatically labeled, quenched, digested, cleaned and separated at 2° C. Samples were digested using an immobilized pepsin column (2.1×30 mm) at 60 μL/min for 2 min followed by an on-line desalting step using a 2 mm I.D×10 mm length C-18 pre-column (ACE HPLC Columns, Aberdeen, UK) using 0.1% formic acid at 400 μl/min for 1 min. Peptic peptides were then separated by a 18 min 8-55% linear gradient of ACN in 0.1% formic acid using a 2 mm I.D×50 mm length HALO 018/1.8 μm analytical column operated at 60 μL/min. An LTQ Elite Orbitrap mass spectrometer (Thermo Fisher Scientific) operated at 120,000 resolution at m/z 400 was use to perform all experiments.

All HDX-MS data was processed by an HDExaminer Version 2.5.1. Mascot that was used for peptide identification in a dedicated database, using a 10 ppm precursor tolerance, 0.05 Da MS/MS mass error.

Results

In the present HDX-MS experiments, IL13Rα2 was labeled with deuterium using a deuterated buffer under two different experimental conditions. One set of experiments using IL13Rα2 alone and another, in which the IL13Rα2 was labelled in the presence of equimolar concentration of W-ME107-117 scFv. After labeling, the reaction was stopped, and the protein was digested by a proteolytic enzyme. In a subsequent step, the individual mass of each deuterated peptide derived from the IL13Rα2 digest was measured by liquid chromatography-mass spectrometry (LC-MS). Under these experimental conditions, differences in deuterium incorporation between IL13Rα2 alone and in the presence of W-ME107-117 scFv could exclusively be attributed to changes in deuterium incorporation caused by the binding of W-ME107-117 scFv. Now, since it is well known in the art that antibodies produce a decrease in deuterium incorporation at the antigen/antibody interface (epitope), differences in deuterium incorporation between these two experimental conditions were determined and mapped into the 3-dimensional (3D) structure of IL13Rα2 with IL-13 (PDB 3LB6, Lupardus, et al., Structure (2010) 18: 332-342) in order to determine the location of the epitope. Peptides derived from the IL13Rα2 with statistically significant difference (t-student test, 3 replicates per experimental condition, and 99% confidence interval, p<0.01) between IL13Rα2 alone and in the presence W-ME107-117 scFv were identified and assigned to belong to the epitope if they were clustered into an area between 15 to 20 Angstrom and show enough solvent exposure to allow the correct binding of the scFv.

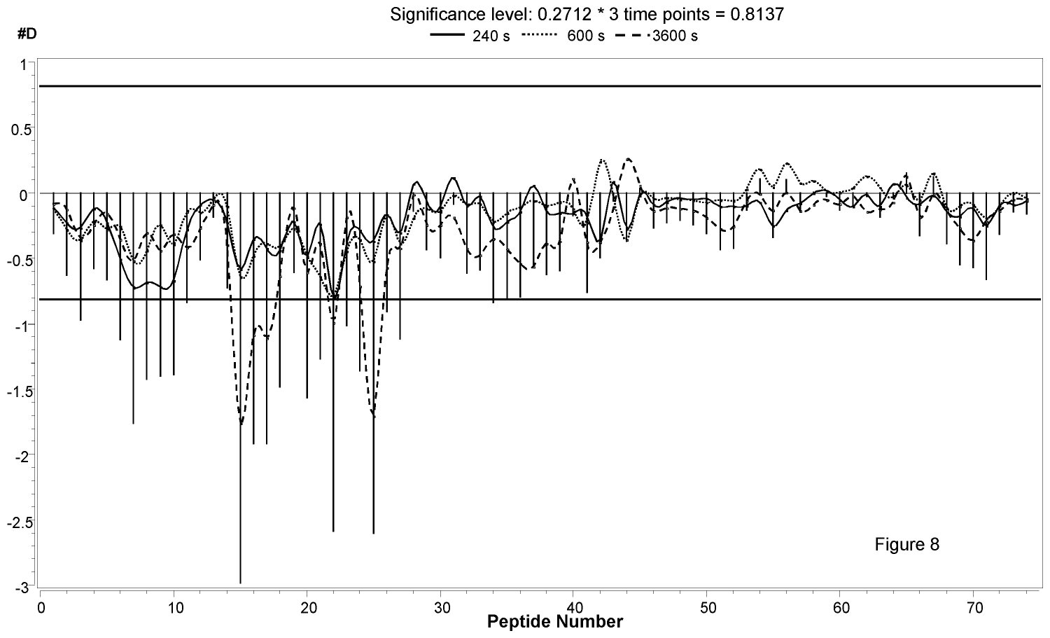

The following peptides were identified as the epitope for W-ME107-117 scFv: amino acid sequence VEYELKYRNIGSETW (SEQ ID NO: 44), corresponding to positions 67-81; amino acid sequence DLNKGIEAKIH (SEQ ID NO: 45), corresponding to positions 96-106; and amino acid sequence WAETTY, (SEQ ID NO: 46) corresponding to positions 123-128 (FIG. 8 and Table 12 and 13). These three sequences mapped to domain 1 of IL13Rα2 and are part of the second beta sheet of this beta sandwich domain. The epitope area more specifically consisted of adjacent beta strands 3, 6 and 7, the loop following beta strand 3 and the loop preceding beta strand 6 (FIG. 10). Both these loops point towards domain 2 of the receptor. The epitope area formed a continuous surface area, which matched those typically seen in antigen/antibody interfaces. The amino acids protruding out from the beta sheet were mainly large amino acids (Trp, Lys, His and Glu) providing several charged groups for potential hydrogen bonding to the antibody. These residues had well defined electron density in the published structure and were seen to form stacking interactions to each other. The epitope area was on the side of domain 1, which did not interact with IL-13.

TABLE 12
Summary of the areas with statistically significant differences
(decrease in deuteration incorporation) between the IL13Rα2 alone
and in the presence of W-ME107-117 (p < 0.001)

| | | | SEQ | Labelling | |
|---|---|---|---|---|---|
| Start | End | Sequence | ID NO: | Time (sec) | P |
| 67 | 80 | VEYELKYRNIGSET | 94 | 600 | 8.33E-05 |
| 67 | 80 | VEYELKYRNIGSET | 94 | 3600 | 2.14E-04 |
| 67 | 80 | VEYELKYRNIGSET | 94 | 240 | 9.14E-04 |
| 69 | 80 | YELKYRNIGSET | 95 | 3600 | 1.96E-04 |
| 71 | 80 | LKYRNIGSET | 96 | 3600 | 4.78E-05 |
| 96 | 109 | DLNKGIEAKIHTLL | 97 | 3600 | 8.73E-04 |
| 103 | 109 | AKIHTLL | 98 | 3600 | 3.92E-04 |
| 103 | 109 | AKIHTLL | 98 | 240 | 4.58E-04 |
| 123 | 128 | WAETTY | 46 | 240 | 4.01E-04 |
| 123 | 128 | WAETTY | 46 | 3600 | 7.69E-04 |
| 228 | 232 | FTFQL | 99 | 3600 | 8.55E-05 |
| 228 | 232 | FTFQL | 99 | 600 | 2.43E-04 |

TABLE 13
Listing of peptides shown in FIG. 8

| Peptide | Start | End |
|---|---|---|
| No. | aa | aa |
| 1 | 29 | 37 |
| 2 | 29 | 38 |
| 3 | 29 | 39 |
| 4 | 30 | 37 |
| 5 | 30 | 39 |
| 6 | 38 | 46 |
| 7 | 38 | 48 |
| 8 | 39 | 48 |
| 9 | 39 | 49 |
| 10 | 40 | 49 |
| 11 | 50 | 64 |
| 12 | 52 | 62 |
| 13 | 63 | 68 |
| 14 | 63 | 69 |
| 15 | 67 | 80 |
| 16 | 69 | 80 |
| 17 | 70 | 80 |
| 18 | 71 | 80 |
| 19 | 74 | 80 |
| 20 | 81 | 95 |
| 21 | 81 | 96 |
| 22 | 81 | 102 |
| 23 | 90 | 95 |
| 24 | 96 | 102 |
| 25 | 96 | 109 |
| 26 | 103 | 109 |
| 27 | 123 | 128 |
| 28 | 146 | 154 |
| 29 | 163 | 171 |
| 30 | 165 | 171 |
| 31 | 165 | 172 |
| 32 | 172 | 179 |
| 33 | 172 | 183 |
| 34 | 173 | 181 |
| 35 | 173 | 183 |
| 36 | 173 | 184 |
| 37 | 173 | 186 |
| 38 | 174 | 183 |
| 39 | 185 | 204 |
| 40 | 187 | 199 |
| 41 | 187 | 202 |
| 42 | 187 | 204 |
| 43 | 187 | 206 |
| 44 | 188 | 206 |
| 45 | 200 | 206 |
| 46 | 203 | 210 |
| 47 | 204 | 210 |
| 48 | 205 | 210 |
| 49 | 205 | 211 |
| 50 | 207 | 211 |
| 51 | 228 | 232 |
| 52 | 228 | 233 |
| 53 | 228 | 243 |
| 54 | 228 | 245 |
| 55 | 229 | 243 |
| 56 | 230 | 243 |
| 57 | 232 | 245 |
| 58 | 232 | 246 |
| 59 | 234 | 243 |
| 60 | 235 | 243 |
| 61 | 235 | 245 |
| 62 | 235 | 246 |
| 63 | 246 | 253 |
| 64 | 253 | 269 |
| 65 | 270 | 275 |
| 66 | 274 | 283 |
| 67 | 274 | 285 |
| 68 | 276 | 283 |
| 69 | 284 | 292 |
| 70 | 323 | 333 |
| 71 | 323 | 337 |
| 72 | 338 | 352 |
| 73 | 339 | 352 |
| 74 | 341 | 352 |

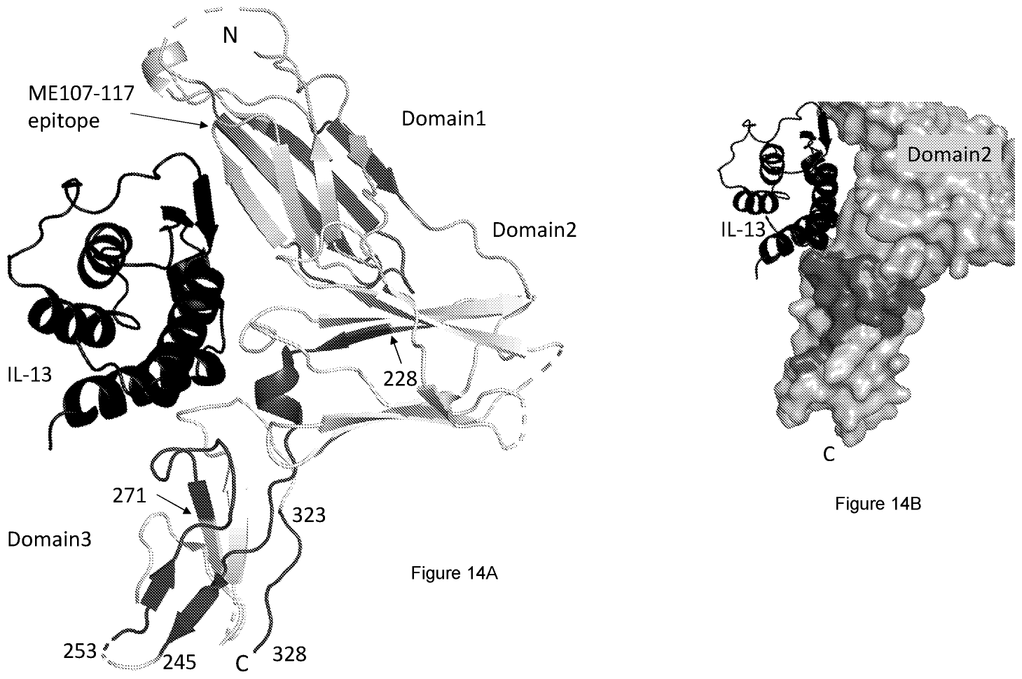

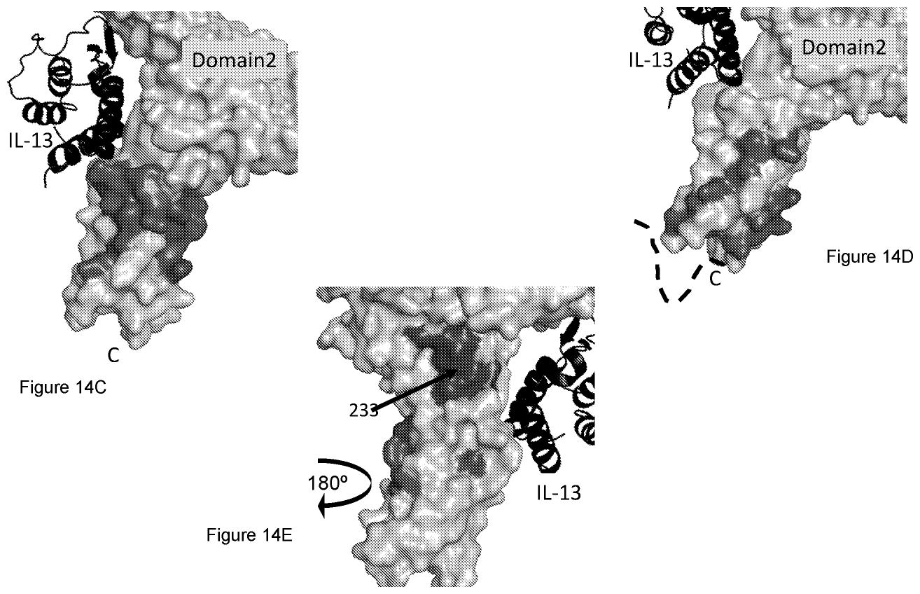

Peptides mapped to clones W-ME107-10, W-ME107-27 and W-ME107-75 were overlapping in different combinations and were all located on domain 3 with the exception of one peptide, which started on domain 2. The peptides are all highlighted in FIG. 14 with the start and stop amino acids marked with numbers (FIG. 14A). HDX-MS of W-ME107-10 gave the following two peptides: FTFQLQNIVKPLPPVYLT (SEQ ID NO: 43) corresponding to amino acid numbers 228-245 and EIKLKWSIPLGPIPARCFD (SEQ ID NO: 36) corresponding to amino acid numbers 253-271. Results for W-ME107-27 gave the same two peptides with an addition of peptide SEWSDKQCWGLNDIF (SEQ ID NO: 23) corresponding to amino acid numbers 323-337 and where residues 332-337 are from the affinity tag of the recombinant protein. The epitope mapped to W-ME107-75 consisted of two peptides with amino acid numbers 253-271 and 323-337 same as mentioned above.

Mapping these three different peptides to the 3-dimensional structure showed that they all cluster towards the C-terminal region of IL13Rα2. The clones W-ME107-10 and W-ME107-27 both had peptide 228-245. This peptide started in domain 2 and continued through to domain 3. Close to the beginning of the peptide, Gln231, Gln233 and Asn234 were in close proximity to the binding site of IL-13 (FIG. 14E), even though none of the residues were seen to interact in the structure. An analysis of the structure of IL13Rα2 indicated that it would be hard for a scFv to interact with these residues and at the same time interact with other peptides mentioned above. A conformational change of IL13Rα2 may explain the inclusion of these residues in the HDX-MS results.

The clone W-ME107-75 lacked the peptide 228-245 in its epitope and was found not to compete with IL-13 binding, whereas the other two clones did. The peptide 253-271 is adjacent to 228-245 and lies towards the IL-13 binding site with Arg268 forming a bond to the ligand. All three clones shared this peptide in the epitope mapping. However, due to the different binding profile of W-ME107-75, it seems unlikely that this scFv will bind peptide 253-271 in the same way as W-ME107-10 and W-ME107-27 but would rather bind more in the beginning of the peptide 253-271 (FIG. 14D).

The peptide 323-337 is the very C-terminal part of the receptor and lies on the opposite side of 228-245. The published structure in Protein Data Bank (PDB) stops at residue 329 and it is not possible to say where the C-terminal part continues. This is most likely a flexible area of the structure. Looking at the data from Examples 5 and 9 of how the different scFv bound mouse IL13Rα2 and then studying where conserved residues were located in the structure gave more ideas to why W-ME107-75 differs from W-ME107-10 and W-ME107-27. There is a band of conserved residues in mouse and human IL13Rα2 that stretches along the top of domain 3 and consists of residues 237-241, 263-269 and 323-326. W-ME107-27 is thought to bind this area as is W-ME107-10 but lacking interactions with peptide 323-326 (FIGS. 14B and 14C). W-ME107-75 on the other hand does not bind as much to this conserved area but rather binds to amino acid residues 253-266 and the very C-terminal residues 332-337. This would mean an area further down domain 3 as compared to W-ME107-10 and W-ME107-27.

Furthermore, the results from the HDX-MS epitope mapping study correlated well with data from Example 12, and analysis of a sequence alignment of mouse and human with binding and non-binding data to the scFv in Example 5 and 9. Clone W-ME107-117 maps to one region of the receptor, which is away from the IL-13 binding site. Clones W-ME107-10, W-ME107-27 and W-ME107-75 were all mapped to similar regions on hIL13Rα2, but W-ME107-10 and W-ME107-27 had one peptide in common, which was lacking in W-ME107-75. This peptide had a small region overlapping with the binding site of the IL-13 ligand. Differences in how these three scFv bind the mouse receptor and where conserved residues are located can also help to pinpoint the different epitopes more.

### Example 14—Epitope Mapping of scFv Using a Peptide Array

Epitope mapping of W-ME107-10, W-ME107-27, W-ME107-75 and W-ME107-117 scFv clones on human IL13Rα2 indicated two distinct parts of the receptor as epitopes for the four clones (Example 13). W-ME107-10, W-ME107-27 and W-ME117-75 seemed to bind similar, or overlapping, regions on domain 3, whereas W-ME107-117 bound a distinct epitope on domain 1. The initial results of the HDX-MS mapping of clone W-ME107-117 resulted in several different peptides spread over both sides of domain 1. In an attempt to narrow down the epitope, 15 aa (amino acid) long peptides with 1 aa in shift covering aa 39-102 on IL13Rα2 were synthesized and assayed in an ELISA-based approach.

No binding of W-ME107-117 scFv to any of the 60 peptides included in the array could be detected. Binding could only be detected towards protein constructs covering the full extracellular domain. This strengthened the finding in Example 13 where it was shown that W-ME107-117 scFv had a conformational epitope rather than a linear peptide epitope in the IL13Rα2 receptor.

Material and Methods

Sixty N-terminally biotinylated peptides, representing a stretch of 74 aa, aa 39-102, of IL13Rα2, were ordered and synthesized by JPT Peptide Technologies (Germany). Upon arrival, peptides were dissolved in sterile DMSO (dimethyl sulfoxide) to a final concentration of 0.5 mg/ml.

A 384-ELISA well plate was coated with 1 μg/ml streptavidin and 1 μg/ml hIL13Rα2-Fc in PBS at 4° C. overnight. Following washing and blocking of the plate in block buffer (PBS supplemented with 0.5% BSA and 0.05% TWEEN 20®), the biotinylated peptides and a biotinylated version of the IL13Rα2 ECD, hIL13Rα2-avi, diluted to 0.25 μg/ml and 1 μg/ml in blocking buffer, respectively, were allowed to bind for 30 minutes. Purified W-ME107-117 and W-ME107-75 were diluted to 1 μg/ml in block buffer and allowed to bind for 1 h.

Detection of binding was enabled through an HRP-conjugated anti-FLAG M2 antibody (Sigma-Aldrich #A8592). Binding signal development was started by adding TMB ELISA substrate (ThermoFisher Scientific #34029) and terminated by the addition of 1 M sulfuric acid. Plates were analyzed at 450 nm.

Each sample was assayed in duplicates from which a mean absorbance value was retrieved after blank well values had been subtracted.

Results

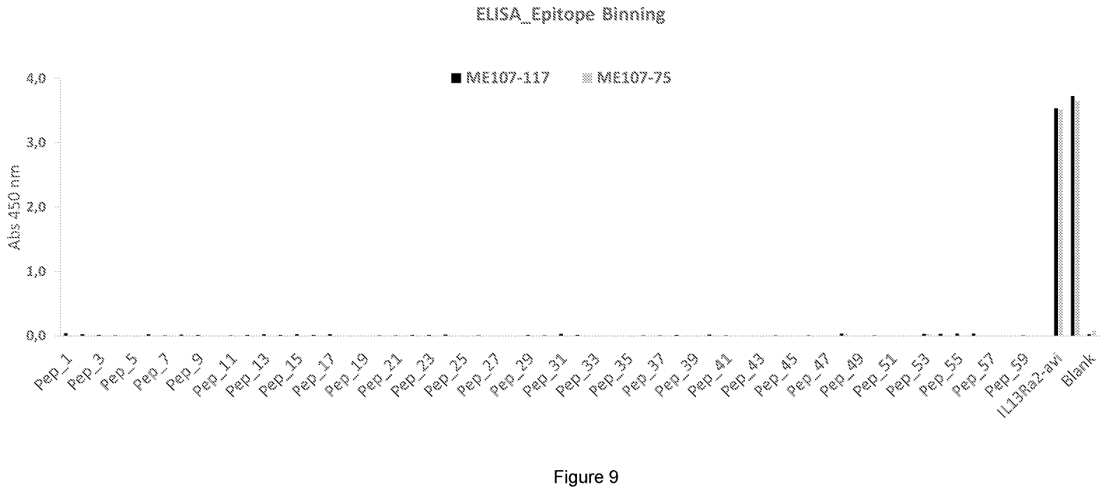

No binding of W-ME107-117 scFv to any of the peptides included in the array could be detected. Binding was only detected towards hIL13Rα2-avi and hIL13Rα2-Fc (FIG. 9). As expected, W-ME107-75 scFv, having an epitope not represented within the peptide array, only displayed binding towards the ECD domain of IL13Rα2. The experiment was repeated using a 10-fold higher concentration of peptides with the same result.

### Conclusion

The epitope mapping study of W-ME107-117 scFv indicated an epitope located on the opposite side of IL13Rα2 from where the epitopes of the W-ME107-10, W-ME107-27 and W-ME107-75 scFv clones were found. The structure depicted in FIG. 10 shows that W-ME107-117 binds a part of IL13Rα2 made up of three beta strands (β-strands) connected by loop regions that fold into a single beta sheet (β-sheet) domain. The β-sheet and the two loops included in the epitope are not in the part of IL13Rα2 that binds the ligand IL-13. Therefore, W-ME107-117 does not interfere with ligand binding. The absence of binding to the peptide array, together with the results from Example 12 strengthens the result of Example 13 where it was found that W-ME107-117 had a conformational epitope.

### Example 15—Lentivirus Construction and T Cell Engineering

This Example constructed chimeric antigen receptor (CAR) T cells based on the selected scFv and tested the CAR T cells in a target cell killing assay.

Materials and Methods

Viral Vector Construction for Transduction of T Cells

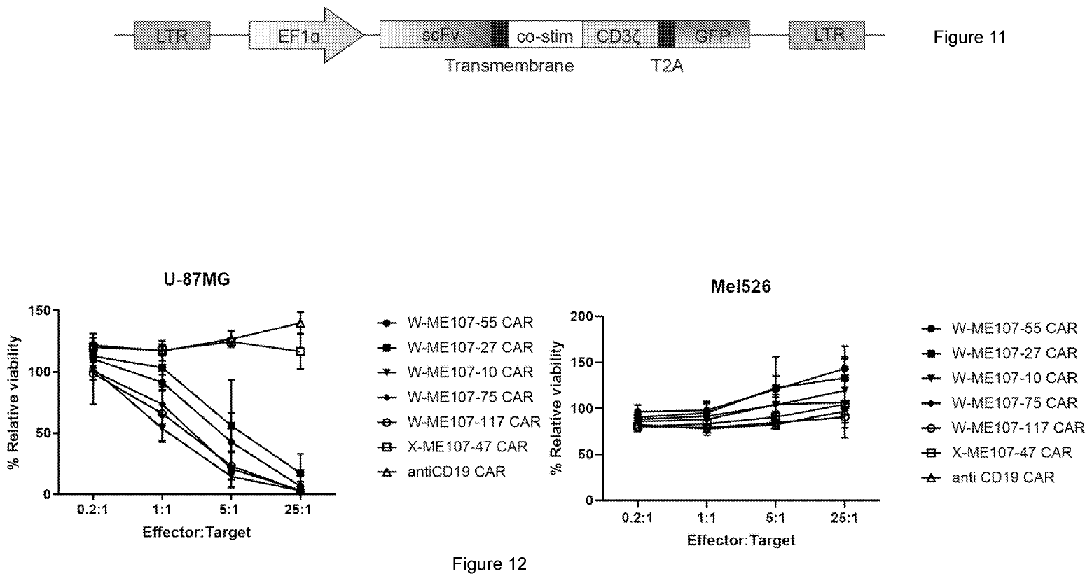

Selected scFv were incorporated into second-generation CAR constructs containing a co-stimulatory domain from CD137 (4-1BB) and a stimulatory domain from CD3ζ. The CAR cassette was cloned into a third generation self-inactivating (SIN) lentiviral vector (SBI, System Biosciences, Mountain View, Calif.) under the control of elongation factor-1 alpha (EF1α) promoter (FIG. 11). Green fluorescent protein (GFP) was incorporated after the CAR cassette and was separated by a self-cleaving T2A sequence. All recombinant sequences were purchased from Genscript (Piscataway, N.J.). Production of viral particles was performed with transient transfection of 293T cells using the constructed CAR lentiviral plasmids and corresponding helper plasmids. Virus particle-containing supernatants were harvested, ultracentrifugation and then used for experiments.

T Cell Engineering

Human peripheral blood mononuclear cells (PBMCs) were activated using OKT-3 (50 ng/ml, BioLegend San Diego, Calif.) and IL-2 (100 IU/ml, Proleukin, Novartis, Basel, Switzerland) for 3 days at a concentration of 2×10⁶ cells per ml. Following activation, T cells (2×10⁶ cells) were re-suspended in 30 μl concentrated lentivirus together with 10 mg/ml protamine sulfate (Sigma-Aldrich, St Louis, Mo.) and IL-2 (100 IU) and incubated for 4 hrs at 37° C. T cells were transduced in a similar manner the day after and re-suspended in culturing medium (RPM11640 supplemented with 10% FBS, 1% PeSt, 1% sodium pyruvate) with a final concentration of 100 IU/ml IL-2. After 7 days CAR T cells were enriched by sorting based on GFP expression (BD FACSAriaIII, BD Bioscience, San Jose, Calif.). Sorted cells were expanded using immunocult reagent (STEMCELL Technologies, Vancouver, Calif.) according to manufacturer's protocol.

Killing Assay

Human glioblastoma cell line U-87MG, naturally expressing hIL13Rα2, and the melanoma cell line Mel526, not expressing hIL13Rα2, were first modified with a lentiviral plasmid to express firefly-luciferase in order to use the luciferase signal as a read out signal of cell viability and exposure to CAR T cells. These target cells were co-cultured with CAR-engineered T cells at effector (CAR T cell) to target (tumor cell) cell ratio 0, 0.2, 1, 5 or 25 CAR T cells to 1 target cell for 24 hrs. CAR T cells and negative control (CD19-targeting CART cells) were used in this Example. Firefly luciferase activity was used as a measurement of target cell viability and was measured using ONE-Glo Luciferase assay system (Promega Biotech AB, Sweden).

All the tested CAR T cell constructs specifically killed the hIL13Rα2-expressing glioblastoma cells (U-87MG) but did not kill the antigen-negative melanoma cells (Mel526) (FIG. 12). Negative control (CD19-targeting CAR T cells) did not kill any of the cancer cell lines, as expected since none of the cancer cell lines express CD19 (FIG. 12).

W-ME107-10 CAR, W-ME107-75 CAR and W-ME107-117 CART cells, based on W-ME107-10 scFv, W-ME107-75 scFv and W-ME107-117 scFv, respectively, displayed substantial cytotoxic capacity already at low effector to target cell ratios. On the other hand, W-ME107-27 CAR and W-ME107-55 CART cells based on W-ME107-27 scFv and W-ME107-55 scFv, demonstrated killing only at a higher effector to target ratio (FIG. 12). Reference X-ME107-47 (mAb47) CAR T cells, produced based on the already published mAb47 scFv (J Biol Chem 2012, 287: 30215-30227, Mol Ther 2016, 24(2): 354-363) showed only marginal target cell killing.

### Example 16—Proliferation Assay

This Example investigated proliferation capacity of various CAR T cells when co-cultured with target cells.

Materials and Methods

The produced CAR T cells were labeled with CellTrace Violet Label (ThermoFisher) in order to follow cell proliferation upon encounter of antigen-positive target cells. CellTrace Violet-Labeled T cells were either left unstimulated or co-cultured with target U-87MG cells (1:1 ratio). Cultures were either left untreated or treated with 10 μM lovastatin (Sigma-Aldrich, to prevent T cell proliferation as a control) for 4 days before being analyzed by flow cytometry (BD FACSCantoll, BD Bioscience). The dilution of violet-dye seen in histograms (peaks pattern) was regarded as cell proliferation.

Results

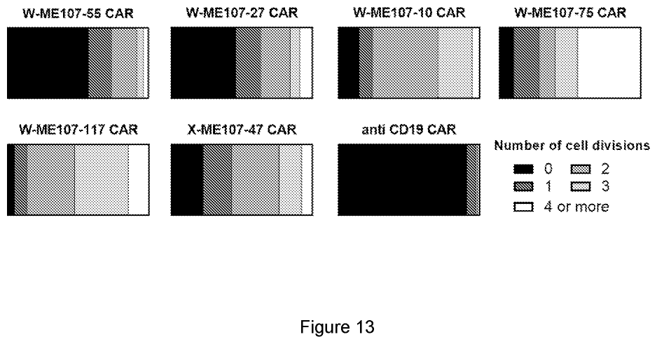

Approximately half of the W-ME107-55 CAR and W-ME107-27 CAR T cells divided upon co-culture with tumor cells. Strikingly, almost all W-ME107-10 CAR, W-ME107-75 CAR and W-ME107-117 CAR T cells divided twice or more upon target cell encounter (FIG. 13). The reference mAb47 (X-ME107-47) CAR T cells also proliferated upon co-culture with target tumor cells, but less than for W-ME107-10 CAR, W-ME107-75 CAR and W-ME107-117 CAR T cells, given the fact that only half the mAb47 CAR T cells divided twice or more. As expected CD19-targeting CART cells did not proliferate in culture with U-87MG cells. Taken together W-ME107-10 CAR, W-ME107-75 CAR and W-ME107-117 CAR T cells demonstrated high proliferative capacity upon target cell recognition. The results show that W-ME107-10 CAR, W-ME107-75 CAR and W-ME107-117 CAR T cells, but not reference mAb47 CAR T cells, were capable of efficiently interacting with the target cells and thereby capable of achieving high proliferative capacity.

### Example 17—CART Cells Profiling and Characterization

This Example investigated and characterized CAR T cells with the cytokine release profile, surface marker expression and CAR expression status.

Materials and Methods

Human CART cells were engineered using lentiviral constructs as described in Example 15. Cells were either cultured in cell culture medium supplemented with IL-2 (25 IU/mL) until analysis or expanded with rapid expansion protocol using 3 donors of PBMCs as stimuli, before analysis.

IFN-Gamma Secretion from Unstimulated CAR T Cells

Mock (as control), W-ME107-55 CAR, W-ME107-27 CAR, W-ME107-10 CAR, W-ME107-75 CAR and W-ME107-117 CAR T cells were engineered using lentiviral vector and cultured in standard T cell culture medium (Example 15). On day 7 after lentivirus transduction, CAR T cells (2×10⁵ cells per well in 200 μL culture medium) were seeded in 96-well plate and cultured for 1 additional day before the supernatant was harvested. IFN-gamma secreted by the CAR T cells into the cell culture supernatant was quantified by ELISA (Mabtech, Sweden).

IFN-Gamma Secretion from Tumor-Stimulated CAR T Cells

Mock (as control), W-ME107-55 CAR, W-ME107-27 CAR, W-ME107-10 CAR, W-ME107-75 CAR and W-ME107-117 CAR T cells were engineered using lentiviral vector (Example 15) expanded using rapid expansion protocol. After expansion, the CAR T cells were rested in cell culture media supplemented with IL-2 (25 IU/mL) for 3 days and CART cells were seeded in 96-well plate together with U87UU or U343MG tumor cells at various ratios and cultured for 2 additional days before the supernatant was harvested. The IFN-gamma secreted into the cell culture supernatant was quantified by ELISA (Mabtech, Sweden).

CAR Expression Level on Cell Surface

Mock (as control), W-ME107-55 CAR, W-ME107-27 CAR, W-ME107-10 CAR, W-ME107-75 CAR and W-ME107-117 CAR T cells were engineered using lentiviral vector and cultured in standard T cell culture medium (Example 15). On day 3, 6 and 12 after lentivirus transduction, CAR T cells were stained for CD3, CAR (using anti-human Ig(H+L) antibody), and analyzed with flow cytometry. CAR expression level overtime was presented as for each CAR T cell as histogram.

CAR T Cell Surface Activation Marker Expression Before and After Tumor Stimulation

Mock (as control), W-ME107-27 CAR, and W-ME107-117 CAR T cells were engineered using lentiviral vector (Example 15) and expanded using rapid expansion protocol. Engineered CAR T cells were rested in cell culture medium supplemented with IL-2 (25 IU/mL) for 3 days before assay. Rested CAR T cells were directly seeded in 96-well plate, cultured alone and analyzed; or co-cultured with U87UU tumor cells for 1 day. The CAR T cells were stained for PD-1, TIM-3, LAG-3, CD69, CD25, and CD3, before analyzed in flow cytometry. Data were presented as percentage of specific-marker positive cells in CAR T cells (gated as CD3-positive and GFP-positive cells).

Results

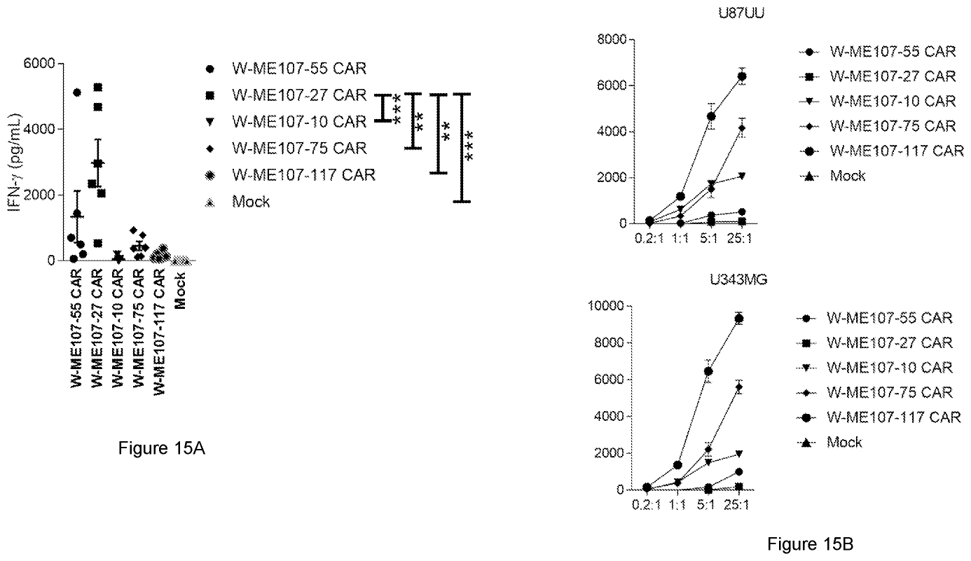

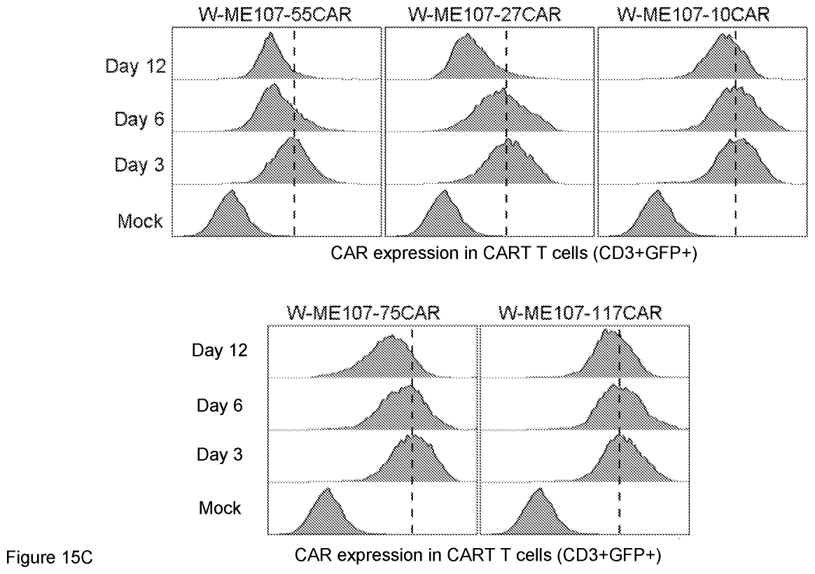

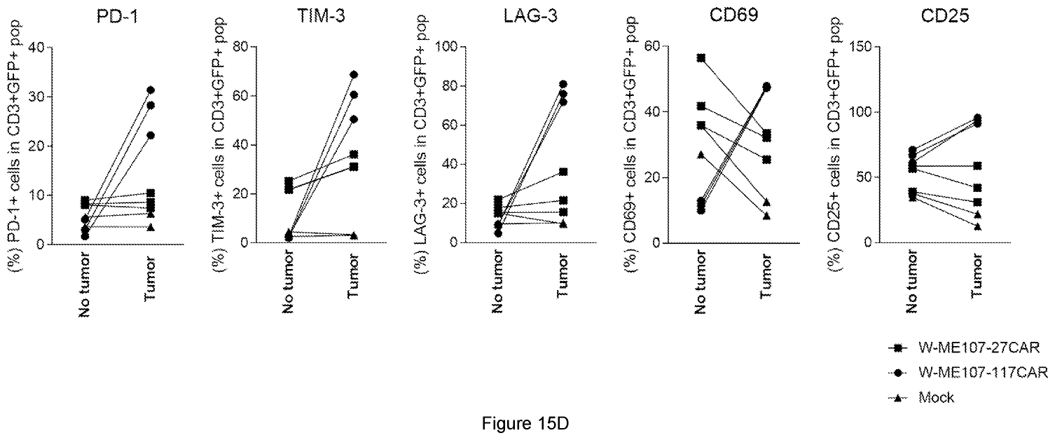

In steady status directly after T cell transduction with lentiviral vectors, W-ME107-55 CAR, and W-ME107-27 CAR T cells secreted high amount of IFN-gamma, while W-ME107-10 CAR, W-ME107-75 CAR and W-ME107-117 CAR T cells secret relatively low amount of IFN-gamma in comparison with Mock CAR T cell control (FIG. 15A). On the other hand, when engineered CAR T cells were co-cultured with tumor cells, W-ME107-10 CAR, W-ME107-75 CAR and W-ME107-117 CAR T cells expressed higher amount of IFN-gamma (IFN-γ) in a dose dependent manner, while W-ME107-55 CAR, and W-ME107-27 CAR T cells barely secret any IFN-gamma in response to tumor cell stimuli (FIG. 15B).

When analyzing surface CAR expression level overtime, we observed that all CAR T cells have the CAR molecules expressed on cell surface after lentivirus transduction. However, expression of W-ME107-55 CAR, and W-ME107-27 CAR decreased overtime, while W-ME107-10 CAR, W-ME107-75 CAR and W-ME107-117 CAR remained at a similar level on cell surface as from beginning (FIG. 15C).

By examining surface activating markers on engineered CAR T cells in the presence and absence of tumor cell stimulation, we noticed that W-ME107-27 CAR T cells had already higher level of these activation markers expressed without tumor cell stimulation, and additional tumor stimulation did not affect the markers expression level. One the other hand, these markers remained at low level in unstimulated W-ME107-117 CAR T cells, while increased their expression drastically upon tumor cell stimulation (FIG. 15D).

All together, these results indicated that W-ME107-55 CAR, and W-ME107-27 CAR T cells had basal level activation that was not target dependent, and this basal level activation led to less responsiveness of the CAR T cells towards target tumor cells.

### Example 18—CART Cells Control Glioblastoma Tumor Growth In Vivo

This Example investigated the efficacy of various CAR T cells in controlling glioblastoma tumor growth in an animal model. Since previous data suggest that W-ME107-55 CAR, and W-ME107-27 CAR T cells had unspecific, target-independent basal level activation, we analyzed the tumor growth inhibitory efficacy of W-ME107-10 CAR, W-ME107-75 CAR and W-ME107-117 CAR T cells.

Materials and Methods

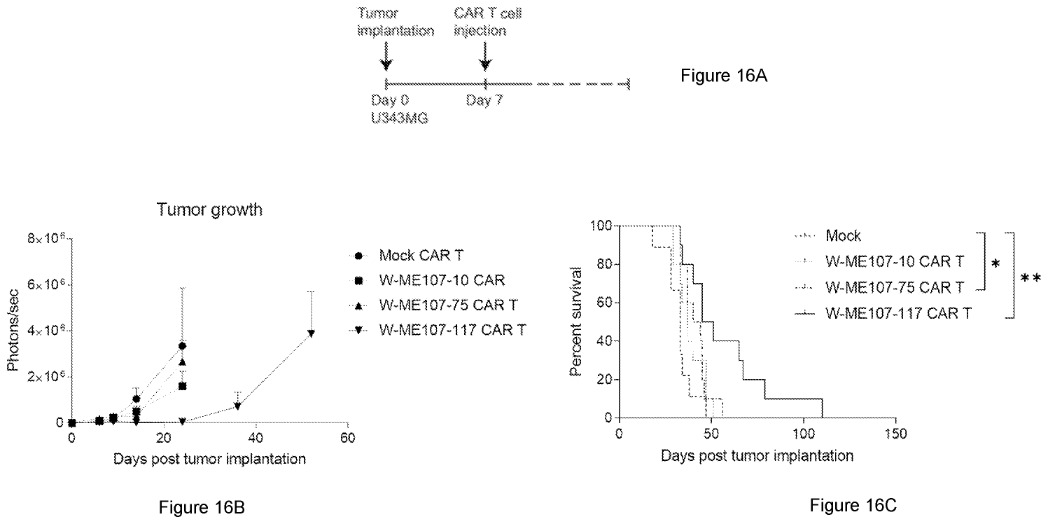

Human CAR T cells were engineered using lentiviral constructs as described in Example 15. Since previous data indicated W-ME107-10 CAR, W-ME107-75 CAR and W-ME107-117 CART were better in proliferation and with lower basal level activation, we evaluated these CAR T cells in vivo. Human glioblastoma cells U343MG-Luc (1×10⁵ cells in 5 μL) were engineered to express fire fly luciferase, and implanted intracranially into nude mice. Injection was performed at 1 mm anterior and 1.5 mm right from bregma and at 2.7 mm depth using a Hamilton syringe and stereotactic injection frame. On day 7 after tumor implantation, mice were treated with Mock T cells (as control) or various CAR T cells (2 million) administrated intracranially at same location. Mice are followed up with imaging of the luciferase signal using IVIS system (NightOWL), and euthanized up on development of severe symptoms strictly following the locally approved animal ethic permission. A schematic experimental procedure is shown in FIG. 16A. The luciferase signal (mean+SEM) was presented as indication of tumor growth, and survival of mice in each treatment groups were presented as Kaplan-Meier curve and compared using Gehan-Breslow-Wilcoxon test.

Results

All three tested CART cells (W-ME107-10 CAR T, W-ME107-75 CART and W-ME107-117 CAR T) could control tumor growth as indicated by lower luciferase signal in the treatment groups compared to Mock T cell treated group (FIG. 16B). In addition, both W-ME107-75 CAR T cell treatment and W-ME107-117 CAR T cell treatment showed significantly improve mice survival in currently assay condition (FIG. 16C).

### Example 19 CDRs Differentially Affect CAR Expression and Functionality

This example investigated the mechanism of CAR expression level differences between different constructs, by substitution of amino acids in the CDR regions with alanine. Since previous data suggested W-ME107-27 and W-ME107-117 had biggest difference in CAR surface expression, we investigated these two clones and substituted amino acids in W-ME107-27 that were different from W-ME107-117.

Material and Methods

Virus Construction and T Cell Engineering

DNA constructs with amino acids substitution (detailed in Table 14) were ordered from Genscript and subcloned into lentiviral vectors. Lentiviral construction was described in Example 15. The human Jurkat T cell line was engineered with the lentiviral constructs. Engineered cells were cultured in cell culture medium (RPM11640 supplemented with 10% FBS, 1% PeSt, 1% sodium pyruvate) until analysis by flow cytometry at 4 days after viral transduction.

TABLE 14
Constructs

| | | SEQ | | SEQ | | SEQ | | SEQ |
|---|---|---|---|---|---|---|---|---|
| Construct | | ID | | ID | | ID | | ID |
| name | HCDR1 | NO: | HCDR2 | NO: | HCDR3 | NO: | LCDR3 | NO: |
| W- | FYGSYMGW | 112 | SYISGYG | 113 | RTPYSAYID | 114 | QFYSYPLT | 115 |
| ME107- | | | | | | | | |
| 27 | | | | | | | | |
| W- | FAGSYMAW | 116 | SYISGYG | 113 | RTPYSAYID | 114 | QFYSYPLT | 115 |
| ME107- | | | | | | | | |
| 27-Ala1 | | | | | | | | |
| W- | FYGSYMGW | 112 | SAIAGAG | 117 | RTPYSAYID | 114 | QFYSYPLT | 115 |
| ME107- | | | | | | | | |
| 27-Ala2 | | | | | | | | |
| W- | FYGSYMGW | 112 | SYISGYG | 113 | RAAAAAYAD | 118 | QFYSYPLT | 115 |
| ME107- | | | | | | | | |
| 27-Ala3 | | | | | | | | |
| W- | FYGSYMGW | 112 | SYISGYG | 113 | RTPYSAYID | 114 | QAYSAPAT | 119 |
| ME107- | | | | | | | | |
| 27-Ala4 | | | | | | | | |
| W- | FAGSYMAW | 116 | SAIAGAG | 117 | PAAAAAYAD | 118 | QAYSAPAT | 119 |
| ME107- | | | | | | | | |
| 27-Ala5 | | | | | | | | |

CAR-Expression Analysis

Engineered cells were stained for CAR (using anti-human Ig(H+L) antibody), and analyzed with flow cytometry. The percentage of CAR-positive cells out of GFP-positive cells were gated and presented.

Results

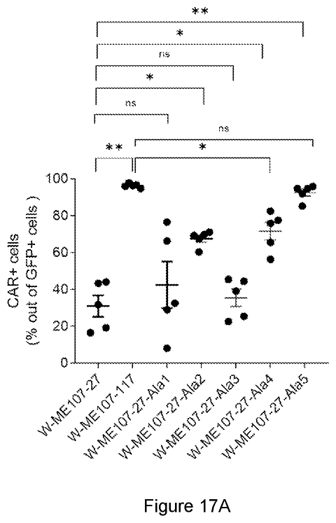

W-ME107-27-Ala2, W-ME107-27-Ala4, W-ME107-27-Ala5 showed significantly higher CAR expression out of GFP positive transduced T cells. W-ME107-27-Ala1, W-ME107-27-Ala3 showed similar level of CAR expression as W-ME107-27 (FIGS. 17A, 17B). These results indicated that CDR2 in the heavy chain and CDR3 in the light chain contributed most in affecting the CAR surface expression.

### Example 20 Basal Level Activation is Associated with Intracellular Signaling Domain

This example investigated the mechanism of basal level activation of engineered CAR T cells, by taking away the intracellular signaling domain of CAR molecules (decoy CARs). Since previous data suggested W-ME107-27 and W-ME107-117 had biggest difference in CAR surface expression, we investigate these two clones.

Material and Methods

Virus Construction and T Cell Engineering

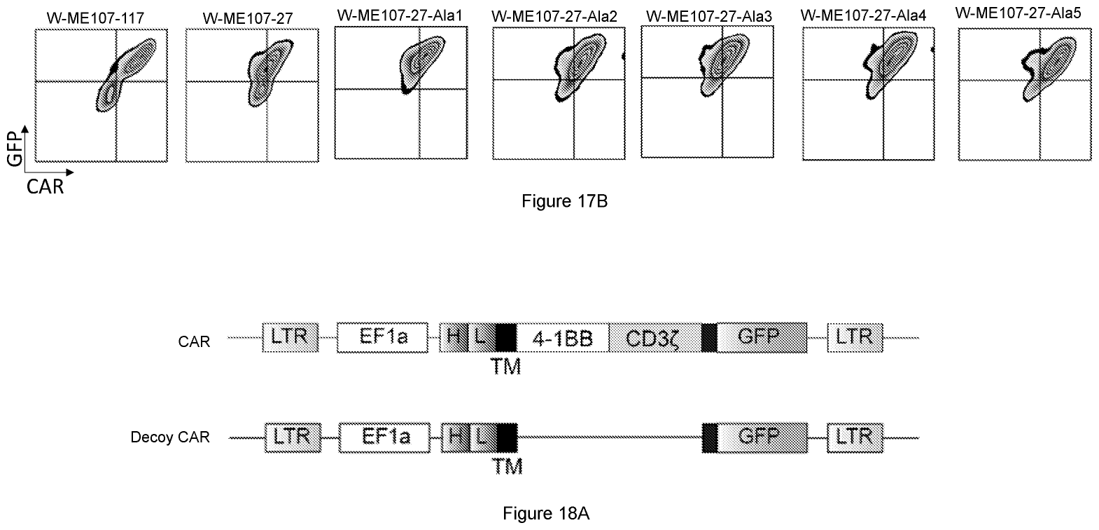

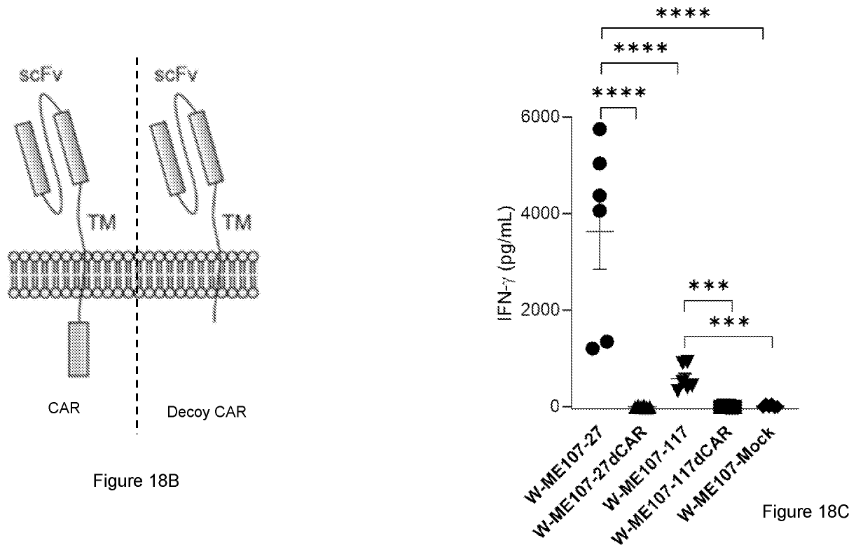

DNA constructs with decoy CAR were ordered form Genscript and subcloned into lentiviral vectors (FIG. 18A), which generated W-ME107-27dCAR and W-ME107-117dCAR. Lentiviral construction was described in Example 15. Human T cells were engineered with lentiviral constructs.

IFN-γ Secretion

Engineered cells were cultured in cell culture medium (RPM11640 supplemented with 10% FBS, 1% PeSt, 1% sodium pyruvate) supplied with 25 IU IL-2/mL for 7 days after viral transduction. Engineered cells (2×10⁵ cells/well) were then plated in 96-well plate in 200 μL cell culture medium without any further cytokine supplement. Cell culture supernatant was harvested 24 hours later and the IFN-γ in the supernatant were measured by ELISA (Mabtech, Sweden).

Results

Both W-ME107-27 and W-ME107-117 CART cells secret IFN-γ at steady status, while W-ME107-27 CAR T cells secreted significantly higher amount of IFN-γ (FIG. 18C). When the intracellular signaling domain was removed, both W-ME107-27dCAR and W-ME107-117dCAR showed significantly lower IFN-γ secretion compared to their counterpart (FIG. 18C). Mock T cells did not secret IFN-γ (FIG. 18C). These results indicated that basal level activation of engineered CART cells is associated with its intracellular signaling domain.

### Example A1—Preparation of Recombinant Human IL13Rα2 (hIL13Rα2-Avi)

Biotinylated hIL13Rα2 was cloned and isolated.

Material and Methods

Material

MultiBac Expression System Kit, Geneva Biotech, NaN

SalI, G|TCGAC Thermo Fischer Scientific, FD0644

XhoI, C|TCGAG Thermo Fischer Scientific, FD0694

KpnI, GGTAC|C Thermo Fischer Scientific, FD0524

PstI, CTGCA|G Thermo Fischer Scientific, FD0615

Rapid DNA Ligation Kit, Thermo Fischer Scientific, K1422

Cre Recombinase, NEB, M0298

GeneJET Plasmid Miniprep Kit, Thermo Fischer Scientific, K0503

PureLink HiPure Plasmid DNA Mini kit, Thermo Fisher Scientific, K210002

LA, Sigma, L7025

LB Broth, Sigma, L03022

One Shot Mach1 T1 Phage-Resistant Chemically Competent E. coli, Thermo Fischer Scientific, C862003

One Shot PIR1 Chemically Competent E. coli, Thermo Fischer Scientific, C101010

Spectamycin, Sigma, S4014

Gentamycin, Sigma, G1397

IPTG, ThermoFisher, 15529019

BluoGal, ThermoFisher, 15519028

Tetracycline, Bioline, 87030

DH10EmBacy competent cells (prepared from multibac kit)

Stellar Competent Cells, Clontech, 636763.

17AEAMOP_ME107h_pMA-T, Gene Art

17AEAMPP_BirA_pMA-T, Gene Art

pFastBac Forward, GGA TTA TTC ATA CCG TCC CA

pACEBac1 Reverse (SV40 polyA), TGA AAT TTG TGA TGC TAT TGC

pIDS Forward, CGA TAC TAG TAT ACG GAC C

pIDS Reverse, CCG TGC GTT TTA TTC TGT C

Hepes, VWR, 441487M

Glycerol, VWR, 444485B

TWEEN 80® (Surfact-Amps 80), Pierce, 0028328

Imidazole, Merck, 1.04716.0250

NaCl, VWR, 27800.360

HisTrap excel 1 ml, GE Healthcare, 17-3712-05

HiLoad Superdex 200 16/60, GE Healthcare, 28-9893-35

Novex Sharp Pre-stained Protein Standard, Invitrogen, LC5800

NuPAGE Antioxidant, Invitrogen, NP0005

NuPAGE LDS Sample Buffer, Invitrogen, NP0007

NuPAGE MES SDS Running Buffer 20×, Invitrogen, NP000202

NuPAGE Novex 4-12% Bis-Tris Protein Gels 1.0 mm 10-well, Invitrogen, NP0321BOX

NuPAGE Novex 4-12% Bis-Tris Protein Gels 1.0 mm 15-well, Invitrogen, NP0323BOX

Pierce Protein Concentrator 10K 5-20 ml, Thermo Scientific, 88527

Anti-6×His tag antibody, Abcam, ab18184

Commassie Protein Assay Reagent, Thermo Fisher Scientific, 1856209

Cloning of hIL13Rα2-avi

The ECD domain of the human IL13Rα2 (Uniprot Q14627 aa 29-331) was co-expressed with the BirA in the baculovirus/Sf9 system to produce the biotinylated receptor. In the gene construct, the native signal peptide (aa 1-28) was exchanged to the gp67 baculovirus signal peptide for secretion of the protein into the culturing medium. An Avi-tag was incorporated C-terminal to the receptor to facilitate directed biotinylation by BirA, followed by a His6-tag for affinity purification. This gave the C-terminal amino acid sequence GLNDIFEAQKIEWHEHHHHHH (SEQ ID NO: 111) added after Trp331. The construct was flanked by the cloning sites SalI and XhoI and codon optimized for Spodoptera frugiperda. Likewise, the E. coli BirA ligase gene was flanked by XhoI and KpnI and codon optimized. The genes were ordered (vector 17AEAMOP_ME107h_pMA-T and vector 17AEAMPP_BirA_pMA-T) from GeneArt, Thermofisher.

The multibac constructs of hIL13Rα2-avi in the acceptor vector pACEBac1 and BirA in donor vector pIDS was made according to manufacturer's protocol. The vector sequences were verified by sequencing at GATC. The two vectors were fused by Cre-Lox recombination, forming the hIL13Rα2-AVIhis/BirA construct. This construct was transformed into DH10EmbacY for transposition into the bacmid. Selection of positive clones was made by blue/white screening in these cells. Finally, the bacmid was isolated and analyzed by PCR for incorporation of genes.

Expression and Characterization of hIL13Rα2-Avi

The bacmid was transfected into Sf9 cells to produce the baculovirus. The human IL13Rα2 was expressed in 2.3 L transfected Sf9 culture during 48 h and harvested from the medium by capturing on HisTrap Excel column. The column was equilibrated with buffer A (50 mM HEPES pH 7.0, 150 mM NaCl, 9 mM imidazole, 10% glycerol and 10 μM TWEEN 80®). After washing the column with buffer A, the protein was eluted with Buffer B (50 mM HEPES pH 7.0, 150 mM NaCl, 9 mM imidazole, 10% glycerol, 10 μM TWEEN 80® and 300 mM imidazole).

After pooling and concentrating the protein containing fractions, the sample was polished on the Superdex 200 16/60 column using 50 mM HEPES pH 7.0, 150 mM NaCl, 10% glycerol and 10 μM TWEEN 80®. The purest protein fractions according to SDS Page were selected, pooled and concentrated. The purified hIL13Rα2 receptor was run on an SDS gel to determine purity and size. Binding to the clone mAb47 single chain control was verified by ELISA and Western blot. The receptor was also sent to Xiaofang Cao, Clinical Proteomics Mass Spectrometry, Science for Life Laboratory for MS analysis. The sample was run three times, but only part of the sequence was covered. We could conclude that the ID of the protein was correct.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1.-51. (canceled)

52. An antibody, or an antigen-binding fragment thereof, capable of binding to interleukin-13 receptor subunit alpha-2 (IL13Rα2), wherein the antibody, or the antigen-binding fragment thereof, comprises:
a variable heavy (VH) domain complementarity determining region 1 (CDR1) consisting of the amino acid sequence GFTFX₁X₂X₃X₄, wherein each Xₙ, n=1 . . . 4, is independently selected from the group consisting of G, A, S and Y;
a VH domain CDR2 consisting of the amino acid sequence IB₁B₂B₃B₄B₅B₆T, wherein each Bₘ, m=1 . . . 6, is independently selected from the group consisting of G, S and Y;
a VH domain CDR3 consisting of the amino acid sequence AR-Z_{H}-Z₁DY, wherein Z₁ is selected from the group consisting of F, M, I and L and Z_{H} represents an amino acid sequence selected from the group consisting of VVRSTYGY (SEQ ID NO: 15), YGHYAYGSY (SEQ ID NO: 16), YSSSGWYYGF (SEQ ID NO: 17), TPYSAY (SEQ ID NO: 18), RYRSHRPGLS (SEQ ID NO: 19), FHPRYGY (SEQ ID NO: 20), GSYSHYGAHY (SEQ ID NO: 21), YYHYDYGYYY (SEQ ID NO: 22), YSPFY (SEQ ID NO: 3), RNYWEHGGGS (SEQ ID NO: 24), HHYGYYPPGSVYY (SEQ ID NO: 25), and VEYTYYGSEGSPV (SEQ ID NO: 26);
a variable light (VL) domain CDR1 consisting of the amino acid sequence QSISSY (SEQ ID NO: 12);
a VL domain CDR2 comprising, preferably consisting of, the amino acid sequence AAS; and
a VL domain CDR3 consisting of the amino acid sequence QQ-Z_{L}-T, wherein Z_{L} represents an amino acid sequence selected from the group consisting of TYYSPH (SEQ ID NO: 28), DYYLF (SEQ ID NO: 29), SYSTPY (SEQ ID NO: 30), FYSYPL (SEQ ID NO: 31), AFSPS (SEQ ID NO: 32), SYDTLL (SEQ ID NO: 33), ALSSLP (SEQ ID NO: 34), FSTRLS (SEQ ID NO: 35), GYSFPP (SEQ ID NO: 4), STYPF (SEQ ID NO: 37), YGSNPL (SEQ ID NO: 38), and RYNGLF (SEQ ID NO: 39).

53. The antibody, or the antigen-binding fragment thereof, according to claim 52, wherein
X₁ is S or Y;
X₂ is S or G;
X₃ is S or Y; and
X₄ is A, Y or G.

54. The antibody, or the antigen-binding fragment thereof, according to claim 52, wherein
B₁ is S or Y;
B₂ is G;
B₃ is S, G or Y;
B₄ is G;
B₅ is S or G; and
B₆ is S or Y.

55. The antibody, or the antigen-binding fragment thereof, according to claim 52, wherein Z_{H}-Z₁ represents an amino acid sequence selected from the group consisting of YGHYAYGSYF (SEQ ID NO: 40), TPYSAYI (SEQ ID NO: 41), GSYSHYGAHYL (SEQ ID NO: 42), and YSPFYM (SEQ ID NO: 9).

56. The antibody, or the antigen-binding fragment thereof, according to claim 52, wherein Z_{L} represents an amino acid sequence selected from the group consisting of DYYLF (SEQ ID NO: 29), FYSYPL (SEQ ID NO: 31), ALSSLP (SEQ ID NO: 34), and GYSFPP (SEQ ID NO: 4).

57. The antibody, or the antigen-binding fragment thereof, according to claim 52, wherein the antibody, or the antigen-binding fragment thereof, comprises:
a VH domain CDR1 consisting of the amino acid sequence of SEQ ID NO: 101, a VH CDR2 consisting of the amino acid sequence of SEQ ID NO: 53; a VH CDR3 consisting of the amino acid sequence of SEQ ID NO: 65, a VL CDR1 consisting of the amino acid sequence of SEQ ID NO: 12, a VL CDR2 consisting of the amino acid sequence of AAS and a VL CDR3 consisting of the amino acid sequence of SEQ ID NO: 76; or
a VH domain CDR1 consisting of the amino acid sequence of SEQ ID NO: 102, a VH CDR2 consisting of the amino acid sequence of SEQ ID NO: 55; a VH CDR3 consisting of the amino acid sequence of SEQ ID NO: 68, a VL CDR1 consisting of the amino acid sequence of SEQ ID NO: 12, a VL CDR2 consisting of the amino acid sequence of AAS and a VL CDR3 consisting of the amino acid sequence of SEQ ID NO: 78; or
a VH domain CDR1 consisting of the amino acid sequence of SEQ ID NO: 104, a VH CDR2 consisting of the amino acid sequence of SEQ ID NO: 54; a VH CDR3 consisting of the amino acid sequence of SEQ ID NO: 70, a VL CDR1 consisting of the amino acid sequence of SEQ ID NO: 12, a VL CDR2 consisting of the amino acid sequence of AAS and a VL CDR3 consisting of the amino acid sequence of SEQ ID NO: 81; or
a VH domain CDR1 consisting of the amino acid sequence of SEQ ID NO: 105, a VH CDR2 consisting of the amino acid sequence of SEQ ID NO: 7; a VH CDR3 consisting of the amino acid sequence of SEQ ID NO: 10, a VL CDR1 consisting of the amino acid sequence of SEQ ID NO: 12, a VL CDR2 consisting of the amino acid sequence of AAS and a VL CDR3 consisting of the amino acid sequence of SEQ ID NO: 11.

58. The antibody, or the antigen-binding fragment thereof, according to claim 52, wherein the antibody, or the antigen-binding fragment thereof, comprises:
a VH domain consisting of the amino acid sequence
(SEQ ID NO: 13)
EVQLLESGGGLVQPGGSLRLSCAASGFTFSGSYMSWVRQAPGKGLEWVSSIYGSGGYT
YYADSVKGRFTISRDNSKNTLYLQMNSLRAEDTAVYYCARYSPFYMDYWGQGTLVTV
SS;
and
a VL domain consisting of the amino acid sequence
(SEQ ID NO: 14)
DIQMTQSPSSLSASVGDRVTITCRASQSISSYLNWYQQKPGKAPKLLIYAASSLQSGVPSR
FSGSGSGTDFTLTISSLQPEDFATYYCQQGYSFPPTFGQGTKLEIK;
or
a VH domain consisting of the amino acid sequence
(SEQ ID NO: 86)
EVQLLESGGGLVQPGGSLRLSCAASGFTFSSYAMSWVRQAPGKGLEWVSAISGSGGSTY
YADSVKGRFTISRDNSKNTLYLQMNSLRAEDTAVYYCARYGHYAYGSYFDYWGQGTL
VTVSS;
and
a VL domain consisting of the amino acid sequence
(SEQ ID NO: 87)
DIQMTQSPSSLSASVGDRVTITCRASQSISSYLNWYQQKPGKAPKLLIYAASSLQSGVPSR
FSGSGSGTDFTLTISSLQPEDFATYYCQQDYYLFTFGQGTKLEIK;
or
a VH domain consisting of the amino acid sequence
(SEQ ID NO: 88)
EVQLLESGGGLVQPGGSLRLSCAASGFTFYGSYMGWVRQAPGKGLEWVSYISGYGGYT
YYADSVKGRFTISRDNSKNTLYLQMNSLRAEDTAVYYCARTPYSAYIDYWGQGTLVTV
SS;
and
a VL domain consisting of the amino acid sequence
(SEQ ID NO: 89)
DIQMTQSPSSLSASVGDRVTITCRASQSISSYLNWYQQKPGKAPKLLIYAASSLQSGVPSR
FSGSGSGTDFTLTISSLQPEDFATYYCQQFYSYPLTFGQGTKLEIK;
or
a VH domain consisting of the amino acid sequence
(SEQ ID NO: 90)
EVQLLESGGGLVQPGGSLRLSCAASGFTFYSYGMSWVRQAPGKGLEWVSYISGGGSYT
YYADSVKGRFTISRDNSKNTLYLQMNSLRAEDTAVYYCARGSYSHYGAHYLDYWGQG
TLVTVSS;
and
a VL domain consisting of the amino acid sequence
(SEQ ID NO: 91)
DIQMTQSPSSLSASVGDRVTITCRASQSISSYLNWYQQKPGKAPKLLIYAASSLQSGVPSR
FSGSGSGTDFTLTISSLQPEDFATYYCQQALSSLPTFGQGTKLEIK;
or
a VH domain consisting of the amino acid sequence
(SEQ ID NO: 13)
EVQLLESGGGLVQPGGSLRLSCAASGFTFSGSYMSWVRQAPGKGLEWVSSIYGSGGYT
YYADSVKGRFTISRDNSKNTLYLQMNSLRAEDTAVYYCARYSPFYMDYWGQGTLVTV
SS;
and
a VL domain consisting of the amino acid sequence
(SEQ ID NO: 14)
DIQMTQSPSSLSASVGDRVTITCRASQSISSYLNWYQQKPGKAPKLLIYAASSLQSGVPSR
FSGSGSGTDFTLTISSLQPEDFATYYCQQGYSFPPTFGQGTKLEIK

59. The antibody, or the antigen-binding fragment thereof, according to claim 52, wherein the antigen-binding fragment is a single-chain variable fragment (scFv).

60. An antibody, or an antigen-binding fragment thereof, capable of binding to interleukin-13 receptor subunit alpha-2 (IL13Rα2), wherein the antibody or the antigen-binding fragment thereof, has specificity for an epitope within a beta sheet area of IL13Rα2 comprising a first beta strand of amino acid number 68 to 75 in IL13Rα2, a loop following the first beta strand, a second beta strand of amino acid numbers 101 to 109 in IL13Rα2, a loop preceding the second beta strand, and a third beta strand of amino acid numbers 124 to 128 in IL13Rα2.

61. The antibody, or the antigen-binding fragment thereof, according to claim 60, wherein the antibody, or the antigen-binding fragment thereof, has specificity for an epitope comprising at least one peptide selected from the group consisting of amino acid number 67 to 81, i.e., VEYELKYRNIGSETW (SEQ ID NO: 44), amino acid number 96 to 106, i.e., DLNKGIEAKIH (SEQ ID NO: 45), and amino acid number 123 to 128, i.e., WAETTY (SEQ ID NO: 46), in IL13Rα2.

62. The antibody, or the antigen-binding fragment thereof, according to claim 60, wherein the antibody, or the antigen-binding fragment thereof, comprises a variable heavy (VH) domain complementarity determining region 3 (CDR3) comprising the amino acid sequence YSPFY (SEQ ID NO: 3).

63. The antibody, or the antigen-binding fragment thereof, according to claim 62, wherein the antibody, or the antigen-binding fragment thereof, comprises a VH domain CDR3 comprising the amino acid sequence YSPFYM (SEQ ID NO: 9).

64. The antibody, or the antigen-binding fragment thereof, according to claim 63, wherein the antibody, or the antigen-binding fragment thereof, comprises a VH domain CDR3 consisting of the amino acid sequence ARYSPFYMDY (SEQ ID NO: 10).

65. The antibody, or the antigen-binding fragment thereof, according to claim 60, wherein the antibody, or the antigen-binding fragment thereof, comprises a variable light (VL) domain complementarity determining region 3 (CDR3) comprising the amino acid sequence GYSFPP (SEQ ID NO: 4).

66. The antibody, or the antigen-binding fragment thereof, according to claim 65, wherein the antibody, or the antigen-binding fragment thereof, comprises a VL domain CDR3 consisting of the amino acid sequence QQGYSFPPT (SEQ ID NO: 11).

67. The antibody, or the antigen-binding fragment thereof, according to claim 60, wherein the antibody, or the antigen-binding fragment thereof, comprises a variable heavy (VH) domain complementarity determining region 1 (CDR1) comprising the amino acid sequence SGSY (SEQ ID NO: 1).

68. The antibody, or the antigen-binding fragment thereof, according to claim 67, wherein the antibody, or the antigen-binding fragment thereof, comprises a VH domain CDR1 consisting of the amino acid sequence GFTFSGSY (SEQ ID NO: 105).

69. The antibody, or the antigen-binding fragment thereof, according to claim 60, wherein the antibody, or the antigen-binding fragment thereof, comprises a variable heavy (VH) domain complementarity determining region 2 (CDR2) comprising the amino acid sequence YGSGGY (SEQ ID NO: 2).

70. The antibody, or the antigen-binding fragment thereof, according to claim 69, wherein the antibody, or the antigen-binding fragment thereof, comprises a VH domain CDR2 consisting of the amino acid sequence IYGSGGYT (SEQ ID NO: 7).

71. The antibody, or the antigen-binding fragment thereof, according to claim 60, wherein the antibody, or the antigen-binding fragment thereof, comprises a variable light (VL) domain complementarity determining region 1 (CDR1) consisting of the amino acid sequence QSISSY (SEQ ID NO: 12).

72. The antibody, or the antigen-binding fragment thereof, according to claim 60, wherein the antibody, or the antigen-binding fragment thereof, comprises a variable light (VL) domain complementarity determining region 2 (CDR2) consisting of the amino acid sequence AAS.

73. The antibody, or the antigen-binding fragment thereof, according to claim 72, wherein the antibody, or the antigen-binding fragment thereof, comprises:
a variable heavy (VH) domain consisting of the
amino acid sequence
(SEQ ID NO: 13)
EVQLLESGGGLVQPGGSLRLSCAASGFTFSGSYMSWVRQAPGKGLEWVS
SIYGSGGYTYYADSVKGRFTISRDNSKNTLYLQMNSLRAEDTAVYYCAR
YSPFYMDYWGQGTLVTVSS;
and
a variable light (VL) domain consisting of the
amino acid sequence
(SEQ ID NO: 14)
DIQMTQSPSSLSASVGDRVTITCRASQSISSYLNWYQQKPGKAPKLLIY
AASSLQSGVPSRFSGSGSGTDFTLTISSLQPEDFATYYCQQGYSFPPTF
GQGTKLEIK.

74. A chimeric antigen receptor (CAR) comprising:
an antigen recognition domain comprising an antibody, or an antigen-binding fragment thereof, according to claim 52;
a transmembrane domain; and
an intracellular signaling domain.

75. The CAR according to claim 74, wherein the transmembrane domain is selected from the group consisting of all, or a portion, of the transmembrane domain of cluster of differentiation 28 (CD28), all, or a portion, of the transmembrane domain of CD8α, all, or a portion, of the transmembrane domain of CD27, all, or a portion, of the transmembrane domain of CD137, all, or a portion, of the transmembrane domain of CD134, all, or a portion, of the transmembrane domain of CD3ε, all, or a portion, of the transmembrane domain of CD3ζ, all, or a portion, of the transmembrane domain of CD3γ, all, or a portion, of the transmembrane domain of CD3δ, all, or a portion, of the transmembrane domain of TCRα, and all, or a portion, of the transmembrane domain of TCRβ.

76. The CAR according to claim 75, wherein the transmembrane domain is selected from the group consisting of all, or a portion, of the transmembrane domain of CD28 and all, or a portion, of the transmembrane domain of CD8α.

77. The CAR according to claim 74, wherein the intracellular signaling domain is selected from the group consisting of zeta chain of cluster of differentiation 3 (CD3ζ), CD28, CD137, ICOS, CD27, CD40, CD134, and Myd88.

78. The CAR according to claim 77, wherein the intracellular signaling domain is selected from the group consisting of CD3ζ and CD137.

79. A T cell receptor (TCR) complex comprising an antigen recognition domain comprising an antibody, or an antigen-binding fragment thereof, according to claim 52.

80. A conjugate comprising:
an antibody, or an antigen-binding fragment thereof, according to claim 52; and
an effector molecule, wherein the effector molecule is preferably selected from the group consisting of a detectable label, a cytotoxin, a metal, another antibody or an antigen-binding fragment thereof, a nucleic acid sequence, and a lipid bilayer docking moiety.

81. The conjugate according to claim 80, wherein the effector molecule is a cytotoxin.

82. A nucleic acid molecule encoding an antibody, or an antigen-binding fragment thereof, according to claim 52.

83. A vector comprising the nucleic acid molecule according to claim 82.

84. A cell comprising an antibody, or an antigen-binding fragment thereof, according to claim 52.

85. The cell according to claim 84, wherein the cell is selected from the group consisting of a T cell, a natural killer (NK) cell, a B cell, a monocyte, and a macrophage.

86. The cell according to claim 85, wherein the cell is a T cell.

87. A pharmaceutical composition comprising an antibody, or an antigen-binding fragment thereof, according to claim 52, and a pharmaceutically acceptable carrier.

88. A method for treating or delaying the onset of a disease selected from the group consisting of glioblastoma, medulloblastoma, breast cancer, head and neck cancer, pancreatic cancer, kidney cancer, ovarian cancer, colon cancer, liver cancer, lung cancer, urothelial cancer and Kaposi's sarcoma in a subject, the method comprises administering an antibody, or an antigen-binding fragment thereof, according to claim 52 to the subject.

89. A method of identifying an interleukin-13 receptor subunit alpha-2 (IL13Rα2)-positive cell comprising:
contacting a biological sample with an antibody, or an antigen-binding fragment thereof, according to claim 52; and
measuring the amount of the antibody, or the antigen-binding fragment thereof, bound to at least one cell of the biological sample, thereby identifying the at least one cell as an IL13Rα2-positive cell.

90. An epitope of interleukin-13 receptor subunit alpha-2 (IL13Rα2), wherein the epitope is within a beta sheet area of IL13Rα2 comprising a first beta strand of amino acid number 68 to 75 in IL13Rα2, a loop following the first beta strand, a second beta strand of amino acid numbers 101 to 109 in IL13Rα2, a loop preceding the second beta strand, and a third beta strand of amino acid numbers 124 to 128 in IL13Rα2.

91. The epitope of claim 90, wherein the epitope comprises at least one peptide selected from the group consisting of amino acid number 67 to 81, i.e., VEYELKYRNIGSETW (SEQ ID NO: 44), amino acid number 96 to 106, i.e., DLNKGIEAKIH (SEQ ID NO: 45), and amino acid number 123 to 128, i.e., WAETTY (SEQ ID NO: 46), in IL13Rα2.


SEQUENCE LISTING (119 sequences)

SEQ ID NO: 1 - protein, 4 aa, Artificial Sequence
  VH CDR1
SGSY

SEQ ID NO: 2 - protein, 6 aa, Artificial Sequence
  VH CDR2
YGSGGY

SEQ ID NO: 3 - protein, 5 aa, Artificial Sequence
  VH CDR3
YSPFY

SEQ ID NO: 4 - protein, 5 aa, Artificial Sequence
  VL CDR3
GYSFP

SEQ ID NO: 5 - protein, 6 aa, Artificial Sequence
  VH CDR1
SGSYMS

SEQ ID NO: 6 - protein, 10 aa, Artificial Sequence
  VH extended CDR1
GFTFSGSYMS

SEQ ID NO: 7 - protein, 8 aa, Artificial Sequence
  VH CDR2
IYGSGGYT

SEQ ID NO: 8 - protein, 10 aa, Artificial Sequence
  VH eCDR2
SIYGSGGYTY

SEQ ID NO: 9 - protein, 6 aa, Artificial Sequence
  VH CDR3
YSPFYM

SEQ ID NO: 10 - protein, 10 aa, Artificial Sequence
  VH CDR3
ARYSPFYMDY

SEQ ID NO: 11 - protein, 9 aa, Artificial Sequence
  VL CDR3
QQGYSFPPT

SEQ ID NO: 12 - protein, 6 aa, Artificial Sequence
  VL CDR1
QSISSY

SEQ ID NO: 13 - protein, 117 aa, Artificial Sequence
  VH chain
EVQLLESGGGLVQPGGSLRLSCAASGFTFSGSYMSWVRQAPGKGLEWVSSIYGSGGYTYYADSVKGRFTISRDNSKNTLYLQMNSLRAEDTAVYYCARYSPFYMDYWGQGTLVTVSS

SEQ ID NO: 14 - protein, 107 aa, Artificial Sequence
  VL chain
DIQMTQSPSSLSASVGDRVTITCRASQSISSYLNWYQQKPGKAPKLLIYAASSLQSGVPSRFSGSGSGTDFTLTISSLQPEDFATYYCQQGYSFPPTFGQGTKLEIK

SEQ ID NO: 15 - protein, 8 aa, Artificial Sequence
  VH CDR3 part
VVRSTYGY

SEQ ID NO: 16 - protein, 9 aa, Artificial Sequence
  VH CDR3 part
YGHYAYGSY

SEQ ID NO: 17 - protein, 10 aa, Artificial Sequence
  VH CDR3 part
YSSSGWYYGF

SEQ ID NO: 18 - protein, 6 aa, Artificial Sequence
  VH CDR3 part
TPYSAY

SEQ ID NO: 19 - protein, 10 aa, Artificial Sequence
  VH CDR3 part
RYRSHRPGLS

SEQ ID NO: 20 - protein, 7 aa, Artificial Sequence
  VH CDR3 part
FHPRYGY

SEQ ID NO: 21 - protein, 10 aa, Artificial Sequence
  VH CDR3 part
GSYSHYGAHY

SEQ ID NO: 22 - protein, 10 aa, Artificial Sequence
  VH CDR3 part
YYHYDYGYYY

SEQ ID NO: 23 - protein, 15 aa, Homo sapiens
SEWSDKQCWGLNDIF

SEQ ID NO: 24 - protein, 10 aa, Artificial Sequence
  VH CDR part
RNYWEHGGGS

SEQ ID NO: 25 - protein, 13 aa, Artificial Sequence
  VH CDR3 part
HHYGYYPPGSVYY

SEQ ID NO: 26 - protein, 13 aa, Artificial Sequence
  VH CDR3 part
VEYTYYGSEGSPV

SEQ ID NO: 27 - protein, 15 aa, Artificial Sequence
  GS-linker
GGGGSGGGGSGGGGS

SEQ ID NO: 28 - protein, 6 aa, Artificial Sequence
  VL CDR3 part
TYYSPH

SEQ ID NO: 29 - protein, 5 aa, Artificial Sequence
  VL CDR3 part
DYYLF

SEQ ID NO: 30 - protein, 6 aa, Artificial Sequence
  VL CDR3 part
SYSTPY

SEQ ID NO: 31 - protein, 6 aa, Artificial Sequence
  VL CDR3 part
FYSYPL

SEQ ID NO: 32 - protein, 5 aa, Artificial Sequence
  VL CDR3 part
AFSPS

SEQ ID NO: 33 - protein, 6 aa, Artificial Sequence
  VL CDR3 part
SYDTLL

SEQ ID NO: 34 - protein, 6 aa, Artificial Sequence
  VL CDR3 part
ALSSLP

SEQ ID NO: 35 - protein, 6 aa, Artificial Sequence
  VL CDR3 part
FSTRLS

SEQ ID NO: 36 - protein, 19 aa, Homo sapiens
EIKLKWSIPLGPIPARCFD

SEQ ID NO: 37 - protein, 5 aa, Artificial Sequence
  VL CDR3 part
STYPF

SEQ ID NO: 38 - protein, 6 aa, Artificial Sequence
  VL CDR3 part
YGSNPL

SEQ ID NO: 39 - protein, 6 aa, Artificial Sequence
  VL CDR3 part
RYNGLF

SEQ ID NO: 40 - protein, 10 aa, Artificial Sequence
  VH CDR3 part
YGHYAYGSYF

SEQ ID NO: 41 - protein, 7 aa, Artificial Sequence
  VH CDR3 part
TPYSAYI

SEQ ID NO: 42 - protein, 11 aa, Artificial Sequence
  VH CDR3 part
GSYSHYGAHYL

SEQ ID NO: 43 - protein, 18 aa, Homo sapiens
FTFQLQNIVKPLPPVYLT

SEQ ID NO: 44 - protein, 15 aa, Homo sapiens
VEYELKYRNIGSETW

SEQ ID NO: 45 - protein, 11 aa, Homo sapiens
DLNKGIEAKIH

SEQ ID NO: 46 - protein, 6 aa, Homo sapiens
WAETTY

SEQ ID NO: 47 - protein, 10 aa, Artificial Sequence
  VH extended CDR1
GFTFGYYYMY

SEQ ID NO: 48 - protein, 10 aa, Artificial Sequence
  VH extended CDR1
GFTFSSYAMS

SEQ ID NO: 49 - protein, 10 aa, Artificial Sequence
  VH extended CDR1
GFTFYGSYMG

SEQ ID NO: 50 - protein, 10 aa, Artificial Sequence
  VH extended CDR1
GFTFGSSYMY

SEQ ID NO: 51 - protein, 10 aa, Artificial Sequence
  VH extended CDR1
GFTFYSYGMS

SEQ ID NO: 52 - protein, 10 aa, Artificial Sequence
  VH extended CDR1
GFTFSSYGMS

SEQ ID NO: 53 - protein, 8 aa, Artificial Sequence
  VH CDR2
ISGSGGST

SEQ ID NO: 54 - protein, 8 aa, Artificial Sequence
  VH CDR2
ISGGGSYT

SEQ ID NO: 55 - protein, 8 aa, Artificial Sequence
  VH CDR2
ISGYGGYT

SEQ ID NO: 56 - protein, 8 aa, Artificial Sequence
  VH CDR2
ISGSGSYT

SEQ ID NO: 57 - protein, 8 aa, Artificial Sequence
  VH CDR2
ISSGSSYT

SEQ ID NO: 58 - protein, 10 aa, Artificial Sequence
  VH eCDR2
AISGSGGSTY

SEQ ID NO: 59 - protein, 10 aa, Artificial Sequence
  VH eCDR2
YISGGGSYTY

SEQ ID NO: 60 - protein, 10 aa, Artificial Sequence
  VH eCDR2
YISGYGGYTY

SEQ ID NO: 61 - protein, 10 aa, Artificial Sequence
  VH eCDR2
GISGSGSYTY

SEQ ID NO: 62 - protein, 10 aa, Artificial Sequence
  VH eCDR2
YISGGGSYTY

SEQ ID NO: 63 - protein, 10 aa, Artificial Sequence
  VH eCDR2
SISSGSSYTY

SEQ ID NO: 64 - protein, 13 aa, Artificial Sequence
  VH CDR3
ARVVRSTYGYFDY

SEQ ID NO: 65 - protein, 14 aa, Artificial Sequence
  VH CDR3
ARYGHYAYGSYFDY

SEQ ID NO: 66 - protein, 15 aa, Artificial Sequence
  VH CDR3
ARYSSSGWYYGFMDY

SEQ ID NO: 67 - protein, 11 aa, Artificial Sequence
  VH CDR3
ARTPYSAYIDY

SEQ ID NO: 68 - protein, 15 aa, Artificial Sequence
  VH CDR3
ARRYRSHRPGLSFDY

SEQ ID NO: 69 - protein, 12 aa, Artificial Sequence
  VH CDR3
ARFHPRYGYFDY

SEQ ID NO: 70 - protein, 15 aa, Artificial Sequence
  VH CDR3
ARGSYSHYGAHYLDY

SEQ ID NO: 71 - protein, 15 aa, Artificial Sequence
  VH CDR3
ARYYHYDYGYYYFDY

SEQ ID NO: 72 - protein, 15 aa, Artificial Sequence
  VH CDR3
ARRNYWEHGGGSLDY

SEQ ID NO: 73 - protein, 18 aa, Artificial Sequence
  VH CDR3
ARHHYGYYPPGSVYYFDY

SEQ ID NO: 74 - protein, 18 aa, Artificial Sequence
  VH CDR3
ARVEYTYYGSEGSPVFDY

SEQ ID NO: 75 - protein, 9 aa, Artificial Sequence
  VL CDR3
QQTYYSPHT

SEQ ID NO: 76 - protein, 8 aa, Artificial Sequence
  VL CDR3
QQDYYLFT

SEQ ID NO: 77 - protein, 9 aa, Artificial Sequence
  VL CDR3
QQSYSTPYT

SEQ ID NO: 78 - protein, 9 aa, Artificial Sequence
  VL CDR3
QQFYSYPLT

SEQ ID NO: 79 - protein, 8 aa, Artificial Sequence
  VL CDR3
QQAFSPST

SEQ ID NO: 80 - protein, 9 aa, Artificial Sequence
  VL CDR3
QQSYDTLLT

SEQ ID NO: 81 - protein, 9 aa, Artificial Sequence
  VL CDR3
QQALSSLPT

SEQ ID NO: 82 - protein, 9 aa, Artificial Sequence
  VL CDR3
QQFSTRLST

SEQ ID NO: 83 - protein, 8 aa, Artificial Sequence
  VL CDR3
QQSTYPFT

SEQ ID NO: 84 - protein, 9 aa, Artificial Sequence
  VL CDR3
QQYGSNPLT

SEQ ID NO: 85 - protein, 9 aa, Artificial Sequence
  VL CDR3
QQRYNGLFT

SEQ ID NO: 86 - protein, 121 aa, Artificial Sequence
  VH chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYGHYAYGSYFDYWGQGTLVTVSS

SEQ ID NO: 87 - protein, 106 aa, Artificial Sequence
  VL chain
DIQMTQSPSSLSASVGDRVTITCRASQSISSYLNWYQQKPGKAPKLLIYAASSLQSGVPSRFSGSGSGTDFTLTISSLQPEDFATYYCQQDYYLFTFGQGTKLEIK

SEQ ID NO: 88 - protein, 118 aa, Artificial Sequence
  VH chain
EVQLLESGGGLVQPGGSLRLSCAASGFTFYGSYMGWVRQAPGKGLEWVSYISGYGGYTYYADSVKGRFTISRDNSKNTLYLQMNSLRAEDTAVYYCARTPYSAYIDYWGQGTLVTVSS

SEQ ID NO: 89 - protein, 107 aa, Artificial Sequence
  VL chain
DIQMTQSPSSLSASVGDRVTITCRASQSISSYLNWYQQKPGKAPKLLIYAASSLQSGVPSRFSGSGSGTDFTLTISSLQPEDFATYYCQQFYSYPLTFGQGTKLEIK

SEQ ID NO: 90 - protein, 122 aa, Artificial Sequence
  VH chain
EVQLLESGGGLVQPGGSLRLSCAASGFTFYSYGMSWVRQAPGKGLEWVSYISGGGSYTYYADSVKGRFTISRDNSKNTLYLQMNSLRAEDTAVYYCARGSYSHYGAHYLDYWGQGTLVTVSS

SEQ ID NO: 91 - protein, 107 aa, Artificial Sequence
  VL chain
DIQMTQSPSSLSASVGDRVTITCRASQSISSYLNWYQQKPGKAPKLLIYAASSLQSGVPSRFSGSGSGTDFTLTISSLQPEDFATYYCQQALSSLPTFGQGTKLEIK

SEQ ID NO: 92 - protein, 274 aa, Artificial Sequence
  1E10B9 scFv
QIQLVQSGPELKKPGETVKIYCKASGYSFRDYSVHWVKQAPGKGLKWMGWINTETGEPTYVDEFKGRFAFFLEASANTVYLQISNLKNEDTATYFCDYRFTYWGQGTLVTVSAGGGGSGGGGSGGGGSEIVMTQTPLILSVTIGQPASISCKSSQSVLYSNGKTYLNWLLQRPGQSPKRLIYLVSKLDSGVPDRFTGSGSGTDFTLKISRVEAEDLGVYYCVQGSHFPYTFGGGTKLEIKAAADYKDHDGDYKDHDIDYKDDDDKAAAHHHHHH

SEQ ID NO: 93 - protein, 279 aa, Artificial Sequence
  mAb47 scFv
QVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSGGGGSGGGGSGGGGSDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKAAADYKDHDGDYKDHDIDYKDDDDKAAAHHHHHH

SEQ ID NO: 94 - protein, 14 aa, Homo sapiens
VEYELKYRNIGSET

SEQ ID NO: 95 - protein, 12 aa, Homo sapiens
YELKYRNIGSET

SEQ ID NO: 96 - protein, 10 aa, Homo sapiens
LKYRNIGSET

SEQ ID NO: 97 - protein, 14 aa, Homo sapiens
DLNKGIEAKIHTLL

SEQ ID NO: 98 - protein, 7 aa, Homo sapiens
AKIHTLL

SEQ ID NO: 99 - protein, 5 aa, Homo sapiens
FTFQL

SEQ ID NO: 100 - protein, 8 aa, Artificial Sequence
  VH CDR1
GFTFGYYY

SEQ ID NO: 101 - protein, 8 aa, Artificial Sequence
  VH CDR1
GFTFSSYA

SEQ ID NO: 102 - protein, 8 aa, Artificial Sequence
  VH CDR1
GFTFYGSY

SEQ ID NO: 103 - protein, 8 aa, Artificial Sequence
  VH CDR1
GFTFGSSY

SEQ ID NO: 104 - protein, 8 aa, Artificial Sequence
  VH CDR1
GFTFYSYG

SEQ ID NO: 105 - protein, 8 aa, Artificial Sequence
  VH CDR1
GFTFSGSY

SEQ ID NO: 106 - protein, 8 aa, Artificial Sequence
  VH CDR1
GFTFSSYG

SEQ ID NO: 107 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 108 - protein, 8 aa, Homo sapiens
EYELKYRN

SEQ ID NO: 109 - protein, 9 aa, Homo sapiens
IEAKIHTLL

SEQ ID NO: 110 - protein, 5 aa, Homo sapiens
AETTY

SEQ ID NO: 111 - protein, 21 aa, Artificial Sequence
  C-terminal with Avi-tag and His6-tag
GLNDIFEAQKIEWHEHHHHHH

SEQ ID NO: 112 - protein, 8 aa, Artificial Sequence
  Heavy CDR1 region
FYGSYMGW

SEQ ID NO: 113 - protein, 7 aa, Artificial Sequence
  Heavy CDR2 region
SYISGYG

SEQ ID NO: 114 - protein, 9 aa, Artificial Sequence
  Heavy CDR3 region
RTPYSAYID

SEQ ID NO: 115 - protein, 8 aa, Artificial Sequence
  Light CDR3 region
QFYSYPLT

SEQ ID NO: 116 - protein, 8 aa, Artificial Sequence
  Heavy CDR1 region
FAGSYMAW

SEQ ID NO: 117 - protein, 7 aa, Artificial Sequence
  Heavy CDR2 region
SAIAGAG

SEQ ID NO: 118 - protein, 9 aa, Artificial Sequence
  Heavy CDR3 region
RAAAAAYAD

SEQ ID NO: 119 - protein, 8 aa, Artificial Sequence
  Light CDR3 region
QAYSAPAT